(19) United States
(12) Patent Application Publication
(10) Pub. No.: US 2015/0176073 A1
(43) Pub. Date: Jun. 25, 2015

(54) USE OF MICROVESICLES IN DIAGNOSIS, PROGNOSIS, AND TREATMENT OF MEDICAL DISEASES AND CONDITIONS

(71) Applicant: Exosome Diagnostics, Inc., Cambridge, MA (US)

(72) Inventor: Johan Karl Olav Skog, Charlestown, MA (US)

(21) Appl. No.: 14/415,990

(22) PCT Filed: Jul. 18, 2013
(86) PCT No.: PCT/US13/51099

§ 371 Date: Jan. 20, 2015

Related U.S. Application Data
(60) Provisional application No. 61/673,024, filed on Jul. 18, 2012.

(51) Int. Cl. C12Q 1/68; C12Q 1/70

(52) U.S. Cl. CPC C12Q 1/6883; C12Q 1/702; C12Q 2600/158; C12Q 2600/112; C12Q 2600/166

(57) ABSTRACT

The invention provides a novel method for detecting the presence or absence of one or more transfer RNAs (tRNAs) contained in microvesicles from a subject. The invention also provides a novel method for detecting the presence or absence of one or more human endogenous retrovirus elements (HERV) in microvesicles from a subject. The methods disclosed is directed to aiding diagnosis, prognosis, minotring and evaluation of a disease or other medical condition in a subject.

### RELATED APPLICATIONS

This application claims the benefit of U.S. Provisional Application No. 61/673,024, filed Jul. 18, 2013. The contents of this application are hereby incorporated by reference in their entirety.

### BACKGROUND

All membrane vesicles shed by cells <0.8 μm in diameter are referred to herein collectively as microvesicles. This may include exosomes, exosome-like particles, prostasomes, dexosomes, texosomes, ectosomes, oncosomes, apoptotic bodies, retrovirus-like particles, and human endogenous retrovirus (HERV) particles. Microvesicles from various cell sources have been extensively studied with respect to protein and lipid content. Recently, microvesicles have been found to also contain both DNA and RNA, including genomic DNA, cDNA, mitochondrial DNA, microRNA (miRNA), and messenger RNA (mRNA). They may facilitate the transfer of genetic information between cells and/or act as a ‘release hatch’ for DNA/RNA/proteins that the cell is trying to eliminate (Mack et al., 2000; Baj-Krzyworzeka et al., 2006; Valadi et al. 2007).

Due to the genetic and proteomic information contained in microvesicles shed by cells, current research is directed at utilizing microvesicles to gain further insight into the status of these cells, for example, disease state or predisposition for a disease.

### SUMMARY OF THE INVENTION

In general, the invention is a novel method for detecting in a subject the presence or absence of a variety of transfer RNAs (tRNAs) contained in microvesicles, thereby aiding the diagnosis, monitoring and evaluation of diseases, other medical conditions, and treatment efficacy.

One aspect of the invention are methods for aiding in the diagnosis, prognosis, or monitoring of a disease or other medical condition in a subject, comprising the steps of: a) isolating a microvesicle fraction from a biological sample from the subject; and b) detecting the presence or absence of one or more tRNAs within the microvesicle fraction, wherein the tRNA is associated with the disease or other medical condition. The methods may further comprise the step or steps of correlating the presence or absence of one or more tRNAs to the presence, absence, or increased or decreased levels of one or more HERV sequences. The methods may also further comprise the step or steps of comparing the result of the detection step to a control (e.g., comparing the levels of one or more tRNAs, HERV sequences, or combinations thereof detected in the sample to the levels of one or more tRNAs, HERV sequences, or combinations thereof in a control sample), wherein the subject is diagnosed as having the disease or other medical condition (e.g., cancer) if there is a measurable difference in the result of the detection step as compared to a control.

In all aspects of the present invention, the tRNAs are RNA and can be identical to, similar to, or fragments of tRNAs. The tRNAs include chromosomal and mitochondrial tRNAs. The tRNAs can be modified post-translationally, for example, aminoacylated.

In all aspects of the present invention, the HERV sequences are RNA and can be identical to, similar to, or fragments of HERV sequences.

In certain preferred embodiments of the foregoing aspects of the invention, the biological sample is a tissue sample or a bodily fluid sample. The biological sample can be cells obtained from a tissue sample or bodily fluid sample. Particularly preferred bodily fluid samples are plasma and serum.

In certain preferred embodiments, the disease or medical condition is associated with the absence or presence one or more tRNAs, HERV sequences, or combinations thereof. In other embodiments, the disease or medical condition is associated with the increased or decreased levels of one or more tRNAs, HERV sequences, or combinations thereof. The absence or presence of one or more tRNAs, HERV sequences, or combinations thereof can be used to diagnose, prognose, or monitor the disease or medical condition. In other embodiments, the increased or decreased levels of one or more tRNAs, HERV sequences, or combinations thereof can be used to diagnose, prognose, or monitor the disease or medical condition.

In certain embodiments of the foregoing aspects of the invention, the disease or other medical condition is a neoplastic disease or condition (e.g., cancer or cell proliferative disorder), a metabolic disease or condition (e.g., diabetes, inflammation, perinatal conditions or a disease or condition associated with iron metabolism), a neurological disease or condition, an immune disorder or condition, a post transplantation condition, a fetal condition, or a pathogenic infection or disease or condition associated with an infection.

Various aspects and embodiments of the invention will now be described in detail. It will be appreciated that modification of the details may be made without departing from the scope of the invention. Further, unless otherwise required by context, singular terms shall include pluralities and plural terms shall include the singular.

All patents, patent applications, and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representations as to the contents of these documents are based on the information available to the applicants and do not constitute any admission as to the correctness of the dates or contents of these documents.

### BRIEF DESCRIPTION OF THE FIGURES

FIG. 1A is a plot showing the size distribution of microvesicle total RNA extracted from 24 mL normal control (subject 1) plasma. Relative fluorescence units (FU) are plotted against the size of RNA (nucleotides, nt). The 25 nt peak represents an internal standard. The most prominent peak represents small RNA.

FIG. 1B is plot showing the size distribution of DNA amplified with prepared cDNA template from microvesicle total RNA. Relative fluorescence units (FU) are plotted against the size of DNA (base pairs, bp). The 15 bp and 1500 bp peaks represent internal standards. Amplified DNA is not detected.

FIG. 1C is plot showing the size distribution of DNA amplified with PCR product (FIG. 1B) from microvesicle total RNA. Relative fluorescence units (FU) are plotted against the size of DNA (base pairs, bp). The 15 bp and 1500 bp peaks represent internal standards. Amplified DNA is not detected.

FIG. 1D is a plot showing the size distribution of DNA amplified with PCR product (FIG. 1C) from microvesicle total RNA. Relative fluorescence units (FU) are plotted against the size of DNA (base pairs, bp). The 15 bp and 1500 bp peaks represent internal standards. Amplified DNA is detected.

FIG. 2A is a plot showing the size distribution of microvesicle total RNA extracted from 2 mL normal control (subject 2) plasma. Relative fluorescence units (FU) are plotted against the size of RNA (nucleotides, nt). The 25 nt peak represents an internal standard. The most prominent peak represents small RNA. The peaks at ˜1900 nt and ˜4700 nt represent 18S and 28S, respectively.

FIG. 2B is plots showing the size distribution of DNA amplified with prepared cDNA from microvesicle total RNA using four different annealing temperatures. Relative fluorescence units (FU) are plotted against the size of DNA (base pairs, bp). Each panel shows a PCR product amplified with different annealing temperature. Top right: 48° C.; Top left: 50° C.; Bottom left: 52° C.; Bottom right: 54° C. The 15 bp and 1500 bp peaks represent internal standards. Amplified DNA is not detected.

FIG. 2C is plots showing the size distribution of DNA amplified with respective PCR product template (FIG. 2B) from microvesicle total RNA. Relative fluorescence units (FU) are plotted against the size of DNA (base pairs, bp). Each panel represents a different PCR product template. Top: 48° C. template; Middle right: 50° C. template; Middle left: 52° C. template; Bottom: 54° C. template. The 15 bp and 1500 bp peaks represent internal standards. Amplified DNA is detected.

FIG. 3A is a plot showing the size distribution of total RNA extracted from 1 mL normal control (subject 1) leukocytes. Relative fluorescence units (FU) are plotted against the size of RNA (nucleotides, nt). The 25 nt peak represents an internal standard. The two most prominent peaks represent 18S (˜1900 nt) and 28S (˜4700 nt). The ˜150 bp peak represents small RNA.

FIG. 3B is a plot showing the size distribution of DNA amplified with prepared cDNA template from leukocyte total RNA. Relative fluorescence units (FU) are plotted against the size of DNA (base pairs, bp). The 15 bp and 1500 bp peaks represent internal standards. Amplified DNA is detected.

FIG. 3C is plots showing the size distribution of DNA amplified with PCR product template (FIG. 3B) from leukocyte total RNA. Relative fluorescence units (FU) are plotted against the size of DNA (base pairs, bp). Each panel represents a different amount of PCR product template. Top left: No dilution; Top right: 1:1 dilution; Bottom: 1:4 dilution. The 15 bp and 1500 bp peaks represent internal standards. Amplified DNA is detected.

FIG. 4A is plots showing the size distribution of microvesicle total RNA extracted from 2 mL serum. Relative fluorescence units (FU) are plotted against the size of RNA (nucleotides, nt). Each panel shows a different subject. Top left: subject 1; Top right: subject 2; Middle left: subject 7; Middle right: subject 5; Bottom left: subject 6; Bottom right: subject 4. The 25 nt peak represents an internal standard. The most prominent peak represents small RNA. The peaks at ˜1900 nt and ˜4700 nt represent 18S and 28S, respectively.

FIG. 4B is plots showing the size distribution of DNA amplified with prepared cDNA template from microvesicle total RNA. Relative fluorescence units (FU) are plotted against the size of DNA (base pairs, bp). Each panel represents a different subject. Top left: subject 1; Top right: subject 2; Middle left: subject 7; Middle right: subject 5; Bottom left: subject 6; Bottom right: subject 4. The 15 bp and 1500 bp peaks represent internal standards. Amplified cDNA is detected in subject 1, 2, 4, and 5. It is thought that amplified genomic DNA is detected in subject 6 and 7.

FIG. 5A is plots showing the size distribution of microvesicle total RNA extracted from 7-8 mL serum. Relative fluorescence units (FU) are plotted against the size of RNA (nucleotides, nt). Each panel represents a different subject. Top right: subject 3; Top left: subject 1; Middle right: subject 2; Middle left: subject 4; Bottom right: subject 5; Bottom left: subject 6. The 25 nt peak represents an internal standard. The most prominent peak represents small RNA. For subjects 4-6, the peaks at ˜1900 nt and ˜4700 nt represent 18S and 28S, respectively. For subjects 1-3, 18S and 28S are incorrectly shown at ˜4,000 nt and ˜7,000 nt, respectively, due to technical difficulties Instead, the 18S and 28S peaks should be shown at ˜1900 nt and at ˜4700 nt, respectively.

FIG. 5B is plots showing the size distribution of DNA amplified with prepared cDNA template from microvesicle total RNA. Relative fluorescence units (FU) are plotted against the size of DNA (base pairs, bp). Each panel represents a different subject. Top left: subject 3; Top right: subject 1; Middle left: subject 2; Middle right: subject 4; Bottom right subject 5; Bottom left: subject 6. Amplified DNA is detected in all subjects.

FIG. 5C is plots showing the size distribution of DNA amplified with PCR products (FIG. 5B) and Illumina adaptors and indexes. Relative fluorescence units (FU) are plotted against the size of DNA (base pairs, bp). Each panel represents a different subject. Top left: subject 3; Top right: subject 1; Middle left: subject 2; Middle right: subject 4; Bottom right subject 5; Bottom left: subject 6. Amplified DNA is detected in all subjects.

### DETAILED DESCRIPTION OF THE INVENTION

Microvesicles are shed by eukaryotic cells, or budded off of the plasma membrane, to the exterior of the cell. These membrane vesicles are heterogeneous in size with diameters ranging from about 10 nm to about 5000 nm. All membrane vesicles shed by cells <0.8 μm in diameter are referred to herein collectively as “microvesicles”. This may include exosomes, exosome-like particles, prostasomes, dexosomes, texosomes, ectosomes, oncosomes, apoptotic bodies, retrovirus-like particles, and human endogenous retrovirus (HERV) particles. Small microvesicles (approximately 10 to 1000 nm, and more often 30 to 200 nm in diameter) that are released by exocytosis of intracellular multivesicular bodies are referred to in the art as “exosomes”. The methods and compositions described herein are equally applicable to microvesicles of all sizes; preferably 30 to 800 nm.

In some of the literature, the term “exosome” also refers to protein complexes containing exoribonucleases which are involved in mRNA degradation and the processing of small nucleolar RNAs (snoRNAs), small nuclear RNAs (snRNAs) and ribosomal RNAs (rRNA) (Liu et al., 2006b; van Dijk et al., 2007). Such protein complexes do not have membranes and are not “microvesicles” or “exosomes” as those terms are used herein.

### Microvesicles Contain HERV Elements and Transfer RNAs

The present invention is related to the discovery that nucleic acids can be isolated from microvesicles obtained from biological samples of subjects, and analysis of these nucleic acids can be useful for diagnosis, prognosis, and monitoring of diseases. The RNA content of microvesicles includes RNAs from the nucleus, cytoplasm, or mitochondria of cells from which the microvesicles originated. Such RNAs can include, but are not limited to, messenger RNAs (mRNA), ribosomal RNA (rRNA), transfer RNA (tRNA), retrotransposon elements, HERV elements, microRNA (miRNA), and other noncoding RNAs. The present invention is primarily concerned with chromosomal tRNAs, mitochondrial tRNAs, and HERV elements.

Approximately 8% (or 98,000 elements and fragments) of the human genome is made up of human endogenous retrovirus (HERV) elements or sequences. These sequences are derived from ancient viral infections from retroviruses that are inherited by successive generations and now are permanently integrated into the genome. Retroviruses are single-stranded RNA viruses that reverse-transcribe their RNA into DNA for integration into the host's genome. Most retroviruses (such as HIV-1) infect somatic cells, but in very rare cases, it is thought that exogenous retroviruses have infected germline cells allowing integrated retroviral genetic sequences to be passed on to subsequent progeny, thereby becoming ‘endogenous’. Endogenous retroviruses have persisted in the genome of their hosts for thousands of years. Once integrated into the host genome, the retroviral genome acquires inactivating mutations during host DNA replication, and therefore becomes defective for replication and infection. Most HERVs are merely traces of original viruses, having first integrated millions of years ago.

HERV elements possess the characteristic provirus structure, including long terminal repeats (LTR), structural proteins (e.g., gag, pol, and env), and a putative primer binding site (PBS) which can be complementary to a distinct transfer RNA (tRNA). Families of HERV elements are designated according to which tRNA they bind. For example, HERV-E family binds tRNA-Glutamic acid (Repaske et al. 1985), while the HERV-H, -I, and -P, respectively bind tRNAs for His, Ile, and Pro (Maeda et al., 1985; Harada et al., 1987).

HERV elements or sequences have been linked to disease and medical conditions. For example, increased transcription of HERV elements has been noted in a number of cancer cell types. Increased expression of these elements in cancer seems to result in part from overall hypomethylation of the genome, which is also associated with genomic instability and tumor progression. Increased expression of HERV RNA and proteins, as well as formation of retrovirus-like particles, has been reported in tumor tissue from breast cancer, melanoma, and germ cell carcinoma. Antibodies against HERV proteins and virus-like particles, are also found in blood of some cancer patients. Recent studies have found that HERV elements are highly enriched in microvesicles released from tumor cells (Balaj et al., 2011).

“HERV elements”, as used herein, refer to RNA sequences that are identical, similar to, or fragments of HERV elements.

tRNAs are generally 73-93 nucleotides in length, and primarily facilitate the translation of messenger RNA (mRNA) into proteins by recognizing the three letter genetic codon and physically transferring the appropriate amino acid for elongation of the protein at the ribosome. Within the human genome, there are 22 mitochondrial tRNA genes, 497 chromosomal tRNA genes, and there are 324 tRNA-derived putative pseudogenes (Lander et al., 2001). In addition to its primary role in protein synthesis, tRNAs are involved in diverse cellular functions including gene expression and cell death regulation (Mei 2010, Wek 1989, Yamasaki 2009), amino acid (Wilcox 1968), lipid (Lennarz 1966), and porphyrin synthesis (Jahn D 1992), protein degradation (Gonda 1989), and retroviral and retrotransposon replication initiation (Dahlberg 1974 (retroviral), Dewannieux 2006 (HERVs)). It is important to take note of tRNAs' unique role in reverse transcription initiation because both retrotransposon and HERV RNAs have been found to be upregulated in cancer microvesicles (Balaj 2010).

tRNAs have also been linked to disease and medical conditions. For example, previous studies have shown that tRNA expression is enhanced in tumor cells (Reviews: White 2004, Marshall 2008) (Kuchino 1978, Winter 2000, Daly 2005, Pavon-Eternod 2009, Zhou 2009). Further, elevated levels of a specific tRNA has been shown to lead to cellular transformation, suggesting a causal role of tRNA in tumorigenesis (Marshall 2008).

Herein we show that microvesicles are enriched in mitochondrial 16S rRNA and mitochondrial tRNA. Some tRNAs present in microvesicles are post-transciptionally modified, such as aminoacylated. Distribution patterns, or abundance of specific tRNAs may be different in microvesicles from a diseased state compared to normal state. Thus, the present invention relates to detection, measuring, analysis, and correlation of tRNA presence, absence, or levels to the diagnosis, prognosis, and monitoring of a disease or other medical condition.

The term “tRNAs”, as used herein, refers to RNA molecules identical to, similar to, or fragments of tRNAs. The tRNA of the present invention can be transcribed from chromosomal DNA, herein referred to as “chromosomal tRNA”, or from mitochondrial DNA, herein referred to as “mitochondrial tRNA”. tRNAs may be found, for example, in the cytoplasm, nucleus, mitochondria, or other organelles and vesicles within a cell. The tRNA of the present invention may also comprise sequences that are complementary to HERV elements or fragments thereof.

As discussed above, HERV elements and tRNAs are differentially expressed in different disease states. Therefore, the presence, absence, or relative levels of tRNAs and/or HERV elements can be used for effective detection, diagnosis, monitoring, and evaluation of a disease or medical condition. For example, elevated levels of a specific species of tRNA associated with elevated expression of a specific HERV element detected in microvesicles isolated from a subject may indicate presence of a disease, such as cancer. Specifically, elevated expression of HERV-H accompanied by higher prevalence of Histidine tRNA may indicate presence of glioma.

Microvesicles as Diagnostic and/or Prognostic Tools

Certain aspects of the present invention are based on the finding that glioblastoma derived microvesicles can be isolated from the serum of glioblastoma patients. These microvesicles contain mRNA associated with tumor cells. The nucleic acids found within these microvesicles, as well as other contents of the microvesicles such as angiogenic proteins, can be used as valuable biomarkers for tumor diagnosis, characterization and prognosis by providing a genetic profile. Contents within these microvesicles can also be used to monitor tumor progression over time by analyzing if other mutations are acquired during tumor progression as well as if the levels of certain mutations are becoming increased or decreased over time or over a course of treatment.

Recently, it has been discovered that tRNAs and HERV elements can be isolated from microvesicles obtained from biological samples. The tRNAs include, but are not limited to, RNA sequences that are identical to, similar to, or fragments of chromosomal and mitochondrial tRNAs. The HERV elements include, but are not limited to, RNA sequences that are identical to, similar to, or fragments of HERV elements.

Certain aspects of the present invention are based on the finding that microvesicles are secreted by tumor cells and circulating in bodily fluids. The number of microvesicles increases as the tumor activity increases. The higher the tumor activity, the higher the concentration of microvesicles in bodily fluids. In addition, the concentration of nucleic acid, in particular small nucleic acid (75-750 nucleotides), increases as the tumor activity increases. Tumor activity may refer to the malignancy, metastatic potential, or proliferation rate of the tumor.

Certain aspects of the present invention are based on another surprising finding that most of the extracellular RNAs in bodily fluid of a subject are contained within microvesicles and thus protected from degradation by ribonucleases.

One aspect of the present invention relates to methods for detecting, diagnosing, monitoring, treating or evaluating a disease or other medical condition in a subject comprising the steps of, isolating microvesicles from a tissue sample or bodily fluid of a subject, and analyzing one or more tRNAs, HERV elements, or combinations thereof contained within the microvesicles. HERV elements and tRNAs are differentially expressed in different disease states. Therefore, the presence, absence, or relative levels of tRNAs and/or HERV elements can be used for effective detection, diagnosis, monitoring, and evaluation of a disease or medical condition. The one or more tRNAs, HERV elements, or combinations thereof are analyzed qualitatively and/or quantitatively, and the results are compared to results expected or obtained for one or more other subjects who have or do not have the disease or other medical condition. The presence of a difference in microvesicular tRNA or HERV element content of the subject, as compared to that of one or more other individuals, can indicate the presence or absence of, the progression of (e.g., changes of tumor size and tumor malignancy), the susceptibility to, or predisposition for a disease or other medical condition in the subject.

Indeed, the isolation methods and techniques described herein provide the following heretofore unrealized advantages: 1) the opportunity to selectively analyze disease or other medical condition-specific tRNAs and/or HERV elements, which may be realized by isolating disease- or medical condition-specific microvesicles apart from other microvesicles within the tissue or fluid sample; and 2) scalability, e.g., to detect tRNAs and/or HERV elements expressed at low levels, the sensitivity can be increased by pelleting more microvesicles from a larger volume of tissue or fluid;

The microvesicles can be isolated from a sample taken from a tissue from a subject. As used herein, a “tissue sample” refers to a sample of tissue isolated from anywhere in the body of the subject, including but not limited to, for example, lung, pancreas, stomach, intestine, bladder, kidney, ovary, testis, skin, colorectal, breast, prostate, brain, esophagus, liver, placenta, spleen, bone marrow, heart, pancreas, lymph node, and combinations thereof. The tissue sample may be isolated from a biopsy tissue or tissue affected by disease or other medical condition, e.g., tumor or cyst.

The microvesicles are preferably isolated from a sample taken of a bodily fluid from a subject. As used herein, a “bodily fluid” refers to a sample of fluid isolated from anywhere in the body of the subject, preferably a peripheral location, including but not limited to, for example, blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intraorgan system fluid, ascitic fluid, tumor cyst fluid, amniotic fluid and combinations thereof. The microvesicles of the present invention are preferably isolated from plasma or serum from a subject.

The term “subject” is intended to include all animals shown to or expected to have microvesicles. In particular embodiments, the subject is a mammal, a human or nonhuman primate, a dog, a cat, a horse, a cow, other farm animals, or a rodent (e.g., mice, rats, guinea pig. etc.). The term “subject” and “individual” are used interchangeably herein.

Methods of isolating microvesicles from a biological sample are known in the art. For example, a method of differential centrifugation is described in a paper by Raposo et al. (Raposo et al., 1996), and similar methods are detailed in the Examples section herein. Methods of anion exchange and/or gel permeation chromatography are described in U.S. Pat. Nos. 6,899,863 and 6,812,023. Methods of sucrose density gradients or organelle electrophoresis are described in U.S. Pat. No. 7,198,923. A method of magnetic activated cell sorting (MACS, Miltenyi) is described in (Taylor and Gercel-Taylor, 2008). A method ofnanomembrane ultrafiltration concentrator is described in (Cheruvanky et al., 2007). Preferably, microvesicles can be identified and isolated from bodily fluid of a subject by a newly developed microchip technology that uses a unique microfluidic platform to efficiently and selectively separate tumor derived microvesicles. This technology, as described in a paper by Nagrath et al. (Nagrath et al., 2007), can be adapted to identify and separate microvesicles using similar principles of capture and separation as taught in the paper. Each of the foregoing references is incorporated by reference herein for its teaching of these methods.

In one embodiment, the microvesicles isolated from a bodily fluid are enriched for those originating from a specific cell type, for example, lung, pancreas, stomach, intestine, bladder, kidney, ovary, testis, skin, colorectal, breast, prostate, brain, esophagus, liver, placenta, fetus cells. Because the microvesicles often carry surface molecules such as antigens from their donor cells, surface molecules may be used to identify, isolate and/or enrich for microvesicles from a specific donor cell type (Al-Nedawi et al., 2008; Taylor and Gercel-Taylor, 2008). In this way, microvesicles originating from distinct cell populations can be analyzed for their RNA content. For example, tumor (malignant and nonmalignant) microvesicles carry tumor-associated surface antigens and may be detected, isolated and/or enriched via these specific tumor-associated surface antigens. In one example, the surface antigen is epithelial-cell-adhesion-molecule (EpCAM), which is specific to microvesicles from carcinomas of lung, colorectal, breast, prostate, head and neck, and hepatic origin, but not of hematological cell origin (Balzar et al., 1999; Went et al., 2004). In another example, the surface antigen is CD24, which is a glycoprotein specific to urine microvesicles (Keller et al., 2007). In yet another example, the surface antigen is selected from a group of molecules CD70, carcinoembryonic antigen (CEA), EGFR, EGFRvIII and other variants, Fas ligand, TRAIL, tranferrin receptor, p38.5, p97 and HSP72. Additionally, tumor specific microvesicles may be characterized by the lack of surface markers, such as CD80 and CD86.

The isolation of microvesicles from specific cell types can be accomplished, for example, by using antibodies, aptamers, aptamer analogs or molecularly imprinted polymers specific for a desired surface antigen. In one embodiment, the surface antigen is specific for a cancer type. In another embodiment, the surface antigen is specific for a cell type which is not necessarily cancerous. One example of a method of microvesicle separation based on cell surface antigen is provided in U.S. Pat. No. 7,198,923. As described in, e.g., U.S. Pat. Nos. 5,840,867 and 5,582,981, WO/2003/050290 and a publication by Johnson et al. (Johnson et al., 2008), aptamers and their analogs specifically bind surface molecules and can be used as a separation tool for retrieving cell type-specific microvesicles. Molecularly imprinted polymers also specifically recognize surface molecules as described in, e.g., U.S. Pat. Nos. 6,525,154, 7,332,553 and 7,384,589 and a publication by Bossi et al. (Bossi et al., 2007) and are a tool for retrieving and isolating cell type-specific microvesicles. Each of the foregoing reference is incorporated herein for its teaching of these methods.

It may be beneficial or otherwise desirable to extract tRNAs and/or HERV elements from the microvesicles prior to the analysis. RNA molecules can be isolated from a microvesicle using any number of procedures, which are well-known in the art, the particular isolation procedure chosen being appropriate for the particular biological sample. Examples of methods for extraction are provided in the Examples section herein. In some instances, with some techniques, it may also be possible to analyze the RNA without extraction from the microvesicle.

In one embodiment, the tRNAs and/or HERV elements are analyzed directly without an amplification step. Direct analysis may be performed with different methods including, but not limited to, the nanostring technology. NanoString technology enables identification and quantification of individual target molecules in a biological sample by attaching a color coded fluorescent reporter to each target molecule. This approach is similar to the concept of measuring inventory by scanning barcodes. Reporters can be made with hundreds or even thousands of different codes allowing for highly multiplexed analysis. The technology is described in a publication by Geiss et al. (Geiss et al., 2008) and is incorporated herein by reference for this teaching.

In another embodiment, it may be beneficial or otherwise desirable to amplify the nucleic acid of the microvesicle prior to analyzing it. Methods of nucleic acid amplification are commonly used and generally known in the art, many examples of which are described herein. If desired, the amplification can be performed such that it is quantitative. Quantitative amplification will allow quantitative determination of relative amounts of the various nucleic acids, to generate a profile as described below.

In one embodiment, the extracted RNA is similar to, identical to, or a fragment of a tRNA. In another embodiment, the extracted RNA is similar to, identical to, or a fragment of a HERV element. RNAs are then preferably reverse-transcribed into complementary DNAs before further amplification. Such reverse transcription may be performed alone or in combination with an amplification step. One example of a method combining reverse transcription and amplification steps is reverse transcription polymerase chain reaction (RT-PCR), which may be further modified to be quantitative, e.g., quantitative RT-PCR as described in U.S. Pat. No. 5,639,606, which is incorporated herein by reference for this teaching.

Nucleic acid amplification methods include, without limitation, polymerase chain reaction (PCR) (U.S. Pat. No. 5,219,727) and its variants such as in situ polymerase chain reaction (U.S. Pat. No. 5,538,871), quantitative polymerase chain reaction (U.S. Pat. No. 5,219,727), nested polymerase chain reaction (U.S. Pat. No. 5,556,773), self sustained sequence replication and its variants (Guatelli et al., 1990), transcriptional amplification system and its variants (Kwoh et al., 1989), Qb Replicase and its variants (Miele et al., 1983), cold-PCR (Li et al., 2008) or any other nucleic acid amplification methods, followed by the detection of the amplified molecules using techniques well known to those of skill in the art. Especially useful are those detection schemes designed for the detection of nucleic acid molecules if such molecules are present in very low numbers. The foregoing references are incorporated herein for their teachings of these methods.

The analysis of nucleic acids present in the microvesicles is quantitative and/or qualitative. For quantitative analysis, the amounts (expression levels), either relative or absolute, of specific nucleic acids of interest within the microvesicles are measured with methods known in the art (described below). For qualitative analysis, the species of specific nucleic acids of interest within the microvesicles, whether wild type or variants, are identified with methods known in the art (described below).

In one embodiment, the tRNA and/or HERV elements are identified and measured from a biological sample in a method comprising: isolating the microvesicle fraction from the biological sample (e.g., by ultracentrifugation), lysing the microvesicles and extracting the RNA (with the optional step of RNA extraction enhancement, e.g., addition of an RNase inhibitor, for example RNAsin), optionally DNase treating the extracted RNA, optionally purifying the extracted RNA (e.g., phenol-chloroform extraction and ethanol precipitation), analyzing RNA quality and concentration, preparing a small RNA cDNA library, amplifying the small RNA cDNA library (e.g., using primers complementary to the 3′ adaptor oligonucleotides), and sequencing the PCR products from the amplification step (e.g., Sanger or Illumina sequencing). Preparing a small RNA cDNA library can include: ligating adaptor oligonucleotides, purifying and concentrating the ligation products, reverse-transcription the ligation product, and purifying and concentrating the cDNA products.

Detection of one or more tRNAs and/or HERV elements can be accomplished by performing a nucleotide variant screen on the nucleic acids within the microvesicles. Such a screen can be as wide or narrow as determined necessary or desirable by the skilled practitioner. It can be a wide screen (set up to detect all tRNAs and/or HERV elements known to be associated with one or more disease states or other medical conditions, e.g., cancer). Where one specific disease or other medical condition is suspected or known to exist, the screen can be specific to that cancer or disease. One example is a brain tumor/brain cancer screen (e.g., set up to detect all tRNAs and/or HERV elements associated with various clinically distinct subtypes of brain cancer or known drug-resistant or drug-sensitive mutations of that cancer).

In one embodiment, the analysis is of a profile of the amounts (levels) of specific nucleic acids present in the microvesicle, herein referred to as a “quantitative nucleic acid profile” of the microvesicles. In another embodiment, the analysis is of a profile of the species of specific nucleic acids present in the microvesicles, herein referred to as a “nucleic acid species profile.” A term used herein to refer to a combination of these types of profiles is “genetic profile” which refers to the determination of the presence or absence of nucleotide species, variants and also increases or decreases in nucleic acid levels.

Once generated, these genetic profiles of the microvesicles are compared to those expected in, or otherwise derived from a healthy normal individual. A profile can be a genome wide profile (set up to detect all possible expressed genes or DNA sequences). It can be narrower as well, such as a cancer wide profile (set up to detect all possible genes or nucleic acids derived therefrom, or known to be associated with one or more cancers).

Where one specific disease or other medical condition is suspected or known to exist, the profile can be specific to that disease or other medical condition (e.g., set up to detect all possible tRNAs or HERV elements derived therefrom, associated with various clinically distinct subtypes of that cancer or known drug-resistant or sensitive mutations of that disease or other medical condition).

Which nucleic acids are to be amplified and/or analyzed can be selected by the skilled practitioner. The entire nucleic acid content of the microvesicles or only a subset of specific nucleic acids which are likely or suspected of being influenced by the presence of a disease or other medical condition such as cancer, can be amplified and/or analyzed. The identification of a nucleic acid aberration(s) in the analyzed microvesicle nucleic acid can be used to diagnose the subject for the presence of a disease such as cancer, hereditary diseases or viral infection with which that aberration(s) is associated. For instance, analysis for the presence or absence of one or more tRNAs or HERV elements specific to a particular disease or other medical condition (e.g., cancer) can indicate the presence of the disease or medical condition in the individual. Alternatively, or in addition, analysis of one or more tRNAs or HERV elements for an increase or decrease in nucleic acid levels specific to a cancer can indicate the presence of the disease or other medical condition in the individual.

The nucleic acid sequences may be complete or partial, as both are expected to yield useful information in diagnosis and prognosis of a disease. The sequences may be sense or anti-sense to the actual gene or transcribed sequences. The skilled practitioner will be able to devise detection methods for a nucleotide variance from either the sense or anti-sense nucleic acids which may be present in a microvesicle. Many such methods involve the use of probes which are specific for the nucleotide sequences which directly flank, or contain the nucleotide variances. Such probes can be designed by the skilled practitioner given the knowledge of the gene sequences and the location of the nucleic acid variants within the gene. Such probes can be used to isolate, amplify, and/or actually hybridize to detect the nucleic acid variants, as described in the art and herein.

Determining the presence or absence of a particular nucleotide variant or plurality of variants in the nucleic acid within microvesicles from a subject can be performed in a variety of ways. A variety of methods are available for such analysis, including, but not limited to, PCR, hybridization with allele-specific probes, enzymatic mutation detection, chemical cleavage of mismatches, mass spectrometry or DNA sequencing, including minisequencing. In particular embodiments, hybridization with allele specific probes can be conducted in two formats: 1) allele specific oligonucleotides bound to a solid phase (glass, silicon, nylon membranes) and the labeled sample in solution, as in many DNA chip applications, or 2) bound sample (often cloned DNA or PCR amplified DNA) and labeled oligonucleotides in solution (either allele specific or short so as to allow sequencing by hybridization). Diagnostic tests may involve a panel of variances, often on a solid support, which enables the simultaneous determination of more than one variance. In another embodiment, determining the presence of at least one nucleic acid variance in the microvesicle nucleic acid entails a haplotyping test. Methods of determining haplotypes are known to those of skill in the art, as for example, in WO 00/04194.

In one embodiment, the determination of the presence or absence of a nucleic acid variant(s) involves determining the sequence of the variant site or sites (the exact location within the sequence where the nucleic acid variation from the norm occurs) by methods such as polymerase chain reaction (PCR), chain terminating DNA sequencing (U.S. Pat. No. 5,547,859), minisequencing (Fiorentino et al., 2003), oligonucleotide hybridization, high-throughput sequencing, mass spectrometry or other nucleic acid sequence detection methods. Methods for detecting nucleic acid variants are well known in the art and disclosed in WO 00/04194, incorporated herein by reference. In an exemplary method, the diagnostic test comprises amplifying a segment of DNA or RNA (generally after converting the RNA to complementary DNA) spanning one or more known variants in the desired gene sequence. This amplified segment is then sequenced and/or subjected to electrophoresis in order to identify transfer RNAs in the amplified segment.

In one embodiment, the invention provides a method of screening for tRNAs and/or HERV elements in the nucleic acids of microvesicles isolated as described herein. This can be achieved, for example, by PCR or, alternatively, in a ligation chain reaction (LCR) (Abravaya et al., 1995; Landegren et al., 1988; Nakazawa et al., 1994). LCR can be particularly useful for detecting point mutations in a gene of interest (Abravaya et al., 1995). The LCR method comprises the steps of designing degenerate primers for amplifying the target sequence, the primers corresponding to one or more conserved regions of the nucleic acid corresponding to the gene of interest, amplifying PCR products with the primers using, as a template, a nucleic acid obtained from a microvesicle, and analyzing the PCR products. Comparison of the PCR products of the microvesicle nucleic acid to a control sample (either having the nucleotide variant or not) indicates variants in the microvesicle nucleic acid. The change can be either an absence or presence of a nucleotide variant in the microvesicle nucleic acid, depending upon the control.

Analysis of amplification products can be performed using any method capable of separating the amplification products according to their size, including automated and manual gel electrophoresis, mass spectrometry, and the like.

Alternatively, the amplification products can be analyzed based on sequence differences, using SSCP, DGGE, TGGE, chemical cleavage, OLA, restriction fragment length polymorphisms as well as hybridization, for example, nucleic acid microarrays.

The methods of nucleic acid isolation, amplification and analysis are routine for one skilled in the art and examples of protocols can be found, for example, in Molecular Cloning: A Laboratory Manual (3-Volume Set) Ed. Joseph Sambrook, David W. Russel, and Joe Sambrook, Cold Spring Harbor Laboratory, 3rd edition (Jan. 15, 2001), ISBN: 0879695773. A particular useful protocol source for methods used in PCR amplification is PCR Basics: From Background to Bench by Springer Verlag; 1st edition (Oct. 15, 2000), ISBN: 0387916008.

Identification of tRNA and/or HERV elements associated with specific diseases and/or medical conditions by the methods described herein can also be used for prognosis and treatment decisions of an individual diagnosed with a disease or other medical condition such as cancer. Presence, absence, or relative levels of tRNAs and/or HERV elements may also provide useful information guiding the treatment of the disease and/or medical condition.

As such, aspects of the present invention relate to a method for monitoring disease (e.g., cancer) progression in a subject, and also to a method for monitoring disease recurrence in an individual. These methods comprise the steps of isolating microvesicles from a tissue or bodily fluid of an individual, as discussed herein, and analyzing nucleic acid within the microvesicles as discussed herein (e.g., to create a genetic profile of the microvesicles). The presence/absence of a certain genetic aberration/profile is used to indicate the presence/absence of the disease or other medical condition (e.g., cancer) in the subject as discussed herein. The process is performed periodically over time, and the results reviewed, to monitor the progression or regression of the disease, or to determine recurrence of the disease. Put another way, a change in the genetic profile indicates a change in the disease state in the subject. The period of time to elapse between sampling of microvesicles from the subject, for performance of the isolation and analysis of the microvesicle, will depend upon the circumstances of the subject, and is to be determined by the skilled practitioner. Such a method would prove extremely beneficial when analyzing a nucleic acid from a gene that is associated with the therapy undergone by the subject. For example, a gene which is targeted by the therapy can be monitored for the development of mutations which make it resistant to the therapy, upon which time the therapy can be modified accordingly. The monitored gene may also be one which indicates specific responsiveness to a specific therapy.

Aspects of the present invention also relate to the fact that a variety of non-cancer diseases and/or medical conditions also are associated with HERV sequences and different levels of tRNAs, and such diseases and/or medical conditions can likewise be diagnosed and/or monitored by the methods described herein. Many such diseases are metabolic, infectious or degenerative in nature. One such disease is diabetes (e.g., diabetes insipidus) in which the vasopressin type 2 receptor (V2R) is modified. Another such disease is kidney fibrosis in which the genetic profiles for the genes of collagens, fibronectin and TGF-13 are changed. Changes in the genetic profile due to substance abuse (e.g., a steroid or drug use), viral and/or bacterial infection, and hereditary disease states can likewise be detected by the methods described herein.

Diseases or other medical conditions for which the inventions described herein are applicable include, but are not limited to, nephropathy, diabetes insipidus, diabetes type I, diabetes II, renal disease glomerulonephritis, bacterial or viral glomerulonephritides, IgA nephropathy, Henoch-Schonlein Purpura, membranoproliferative glomerulonephritis, membranous nephropathy, Sjogren's syndrome, nephrotic syndrome minimal change disease, focal glomerulosclerosis and related disorders, acute renal failure, acute tubulointerstitial nephritis, pyelonephritis, GU tract inflammatory disease, Pre-clampsia, renal graft rejection, leprosy, reflux nephropathy, nephrolithiasis, genetic renal disease, medullary cystic, medullar sponge, polycystic kidney disease, autosomal dominant polycystic kidney disease, autosomal recessive polycystic kidney disease, tuberous sclerosis, von Hippel-Lindau disease, familial thin-glomerular basement membrane disease, collagen III glomerulopathy, fibronectin glomerulopathy, Alport's syndrome, Fabry's disease, Nail-Patella Syndrome, congenital urologic anomalies, monoclonal gammopathies, multiple myeloma, amyloidosis and related disorders, febrile illness, familial Mediterranean fever, HIV infection-AIDS, inflammatory disease, systemic vasculitides, polyarteritis nodosa, Wegener's granulomatosis, polyarteritis, necrotizing and crecentic glomerulonephritis, polymyositis-dermatomyositis, pancreatitis, rheumatoid arthritis, systemic lupus erythematosus, gout, blood disorders, sickle cell disease, thrombotic thrombocytopenia purpura, Fanconi's syndrome, transplantation, acute kidney injury, irritable bowel syndrome, hemolytic-uremic syndrome, acute corticol necrosis, renal thromboembolism, trauma and surgery, extensive injury, burns, abdominal and vascular surgery, induction of anesthesia, side effect of use of drugs or drug abuse, circulatory disease myocardial infarction, cardiac failure, peripheral vascular disease, hypertension, coronary heart disease, non-atherosclerotic cardiovascular disease, atherosclerotic cardiovascular disease, skin disease, psoriasis, systemic sclerosis, respiratory disease, COPD, obstructive sleep apnoea, hypoxia at high altitude or erdocrine disease, acromegaly, diabetes mellitus, or diabetes insipidus.

Selection of an individual from whom the microvesicles are isolated is performed by the skilled practitioner based upon analysis of one or more of a variety of factors. Such factors for consideration are whether the subject has a family history of a specific disease (e.g., a cancer), has a genetic predisposition for such a disease, has an increased risk for such a disease due to family history, genetic predisposition, other disease or physical symptoms which indicate a predisposition, or environmental reasons. Environmental reasons include lifestyle, exposure to agents which cause or contribute to the disease such as in the air, land, water or diet. In addition, having previously had the disease, being currently diagnosed with the disease prior to therapy or after therapy, being currently treated for the disease (undergoing therapy), being in remission or recovery from the disease, are other reasons to select an individual for performing the methods.

The methods described herein are optionally performed with the additional step of selecting a gene or nucleic acid for analysis, prior to the analysis step. This selection can be based on any predispositions of the subject, or any previous exposures or diagnosis, or therapeutic treatments experienced or concurrently undergone by the subject.

The cancer diagnosed, monitored or otherwise profiled, can be any kind of cancer. This includes, without limitation, epithelial cell cancers such as lung, ovarian, cervical, endometrial, breast, brain, colon and prostate cancers. Also included are gastrointestinal cancer, head and neck cancer, non-small cell lung cancer, cancer of the nervous system, kidney cancer, retina cancer, skin cancer, liver cancer, pancreatic cancer, genital-urinary cancer and bladder cancer, melanoma, and leukemia. In addition, the methods and compositions of the present invention are equally applicable to detection, diagnosis and prognosis of non-malignant tumors in an individual (e.g., neurofibromas, meningiomas and schwannomas).

In one embodiment, the cancer is brain cancer. Types of brain tumors and cancer are well known in the art. Glioma is a general name for tumors that arise from the glial (supportive) tissue of the brain. Gliomas are the most common primary brain tumors. Astrocytomas, ependymomas, oligodendrogliomas, and tumors with mixtures of two or more cell types, called mixed gliomas, are the most common gliomas. The following are other common types of brain tumors: Acoustic Neuroma (Neurilemmoma, Schwannoma. Neurinoma), Adenoma, Astracytoma, Low-Grade Astrocytoma, giant cell astrocytomas, Mid- and High-Grade Astrocytoma, Recurrent tumors, Brain Stem Glioma, Chordoma, Choroid Plexus Papilloma, CNS Lymphoma (Primary Malignant Lymphoma), Cysts, Dermoid cysts, Epidermoid cysts, Craniopharyngioma, Ependymoma Anaplastic ependymoma, Gangliocytoma (Ganglioneuroma), Ganglioglioma, Glioblastoma Multiforme (GBM), Malignant Astracytoma, Glioma, Hemangioblastoma, Inoperable Brain Tumors, Lymphoma, Medulloblastoma (MDL), Meningioma, Metastatic Brain Tumors, Mixed Glioma, Neurofibromatosis, Oligodendroglioma. Optic Nerve Glioma, Pineal Region Tumors, Pituitary Adenoma, PNET (Primitive Neuroectodermal Tumor), Spinal Tumors, Subependymoma, and Tuberous Sclerosis (Bourneville's Disease).

In addition to identifying previously known HERV sequences and tRNAs (as associated with diseases), the methods of the present invention can be used to identify previously unidentified HERV sequences and tRNAs or modifications thereof (e.g., post transcriptional modifications) that are associated with a certain disease and/or medical condition. This is accomplished, for example, by analysis of the nucleic acid within microvesicles from a bodily fluid of one or more subjects with a given disease/medical condition (e.g., a clinical type or subtype of cancer) and comparison to the nucleic acid within microvesicles of one or more subjects without the given disease/medical condition, to identify differences in their nucleic acid content. The differences may include, without limitation, expression level of the nucleic acid, alternative splice variants, gene copy number variants (CNV), modifications of the nucleic acid, single nucleotide polymorphisms (SNPs), and mutations (insertions, deletions or single nucleotide changes) of the nucleic acid. Once a difference in a genetic parameter of a particular nucleic acid is identified for a certain disease, further studies involving a clinically and statistically significant number of subjects may be carried out to establish the correlation between the genetic aberration of the particular nucleic acid and the disease. The analysis of genetic aberrations can be done by one or more methods described herein, as determined appropriate by the skilled practitioner.

### EXAMPLES

### Example 1

In one embodiment, plasma was isolated from a normal control (subject 1). Plasma was filtered through a 0.8 μm filter and divided into 1 mL aliquots. Aliquots were frozen at −80° C. until needed.

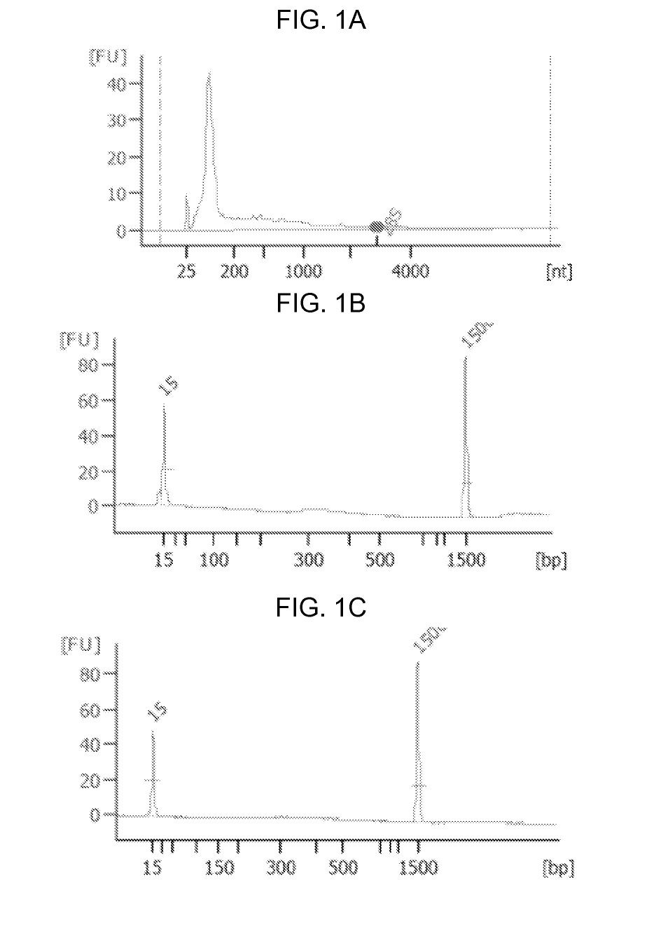

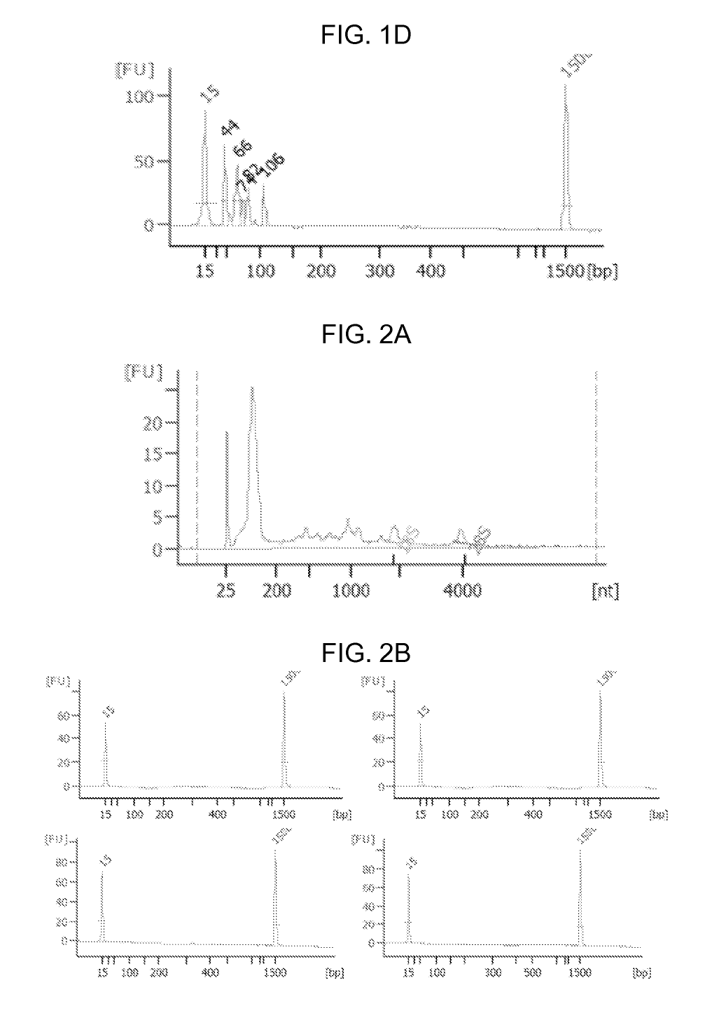

Isolation of microvesicle RNA was conducted using twenty-four 1 mL aliquots of subject 1 plasma. The plasma was evenly split into eight 3 mL aliquots, transferred to 5 mL polyallomer tubes (Beckman-Coulter, Miami, Fl., USA) containing 8 μL RNasin Plus (40 u/μl, Promega, Madison, Wi., USA) RNase inhibitor, and incubated for 5 min at room temp. Following incubation, the plasma aliquots were diluted in 2 mL PBS. Microvesicles were pelleted by ultracentrifugation at 120,000 g for 80 minutes. The microvesicle pellets were each washed in 42 μL PBS and 8 μL RNasin Plus, and incubated for 20 minutes at room temp. Microvesicle pellets were lysed in 700 ul Qiazol Reagent (Qiagen, Valencia, Ca., USA) and isolated using the miRNeasy kit (Qiagen). All eight aliquots of total RNA were combined, and concentrated and purified using a 30 kDa centrifugal filter unit (Millipore, Bedford, Ma., USA). The total RNA was further concentrated to 10 μL in a Speed Vac concentrator (Savant, Farmingdale, Ny., USA). Following concentration, the total RNA was quantified using a nanodrop ND-2000 instrument (Thermo Fischer Scientific, Wilmington, De., USA). Subject 1 plasma microvesicles were found to contain 1.6 ng RNA/mL plasma. The total RNA was further purified by phenol-chloroform extraction and ethanol precipitation. RNA quality and concentration was assessed with the 2100 Bioanalyzer (Agilent, Palo Alto, Ca., USA) using a RNA 6000 Pico Chip (FIG. 1A).

Small RNA cDNA library preparation was performed as previously described (Pak) with modifications. Total microvesicle RNA was ligated at 37° C. for 1 hour and 16° C. for 16 hours to a 5′-adenylated 3′-adaptor oligonucleotide (5′-rAppCTGTAGGCACCATCAAT/ddC/-3′ (SEQ ID NO: 1) (IDT DNA, Coralvill, Ia., USA) at a 1:60 molar ratio in a reaction volume of 20 μl containing 10 U T4 RNA Ligase I (10 U/μL; New England BioLabs, Beverly, Ma., USA), 20 U RNasin Plus (Promega), and 1×T4 RNA Ligase Buffer (New England BioLabs). After ligation, the product was purified by phenol-chloroform extraction, filtered using a 30 kDa centrifugal filter unit (Millipore) to remove the 3′-adaptor oligonucleotide, and concentrated to 11 μL in a Speed Vac concentrator (Savant). The ligation product was reverse transcribed using Sensiscript (Qiagen) and a primer with sequence complementarity to the 3′-adaptor oligonucleotide (5′-GATTGATGGTGCCTACAG-3′ (SEQ ID NO: 2) (IDT DNA)). Following reverse transcription, the cDNA was purified by phenol-chloroform extraction, filtered using a 30 kDa centrifugal filter unit (Millipore) to remove the primer, and concentrated to 13 μL in a Speed Vac concentrator (Savant). The cDNA was ligated at 37° C. for 1 hour and 16° C. for 2 hours to a second 5′-adenylated 3′-adaptor oligonucleotide (5′-rAppCACTCGGGCACCAAGGA/ddC/-3′ (SEQ ID NO: 3) (IDT DNA)) at a 1:60 molar ratio in a reaction volume of 20 μl containing 10 U T4 RNA Ligase I (New England BioLabs), 5% DMSO, and 1×T4 RNA Ligase Buffer (New England BioLabs). After ligation, the cDNA was purified by phenol-chloroform extraction, filtered using a 30 kDa centrifugal filter unit (Millipore) to remove the second 5′-adenylated 3′-adaptor oligonucleotide, and concentrated to 9 μL in a Speed Vac concentrator (Savant).

The final ligation product was PCR amplified using the reverse transcription primer and a primer with sequence complementarity to the second 3′-adaptor (5′-GTCCTTGGTGCCCGAGTG-3′ (SEQ ID NO: 4) (IDT DNA)) in a reaction volume of 20 μL containing 1 U Platinum Taq DNA Polymerase (Invitrogen, Carlsbad, Ca., USA), 1× Platinum Taq DNA Polymerase Buffer, 3 mM MgCl₂, 0.5 mM dNTPs, and 0.5 μM of each primer. Amplification conditions consisted of: 1 cycle of 95° C., 10 min; 40 cycles of 95° C. 30 sec; 50° C. 30 sec; 68° C. 30 sec; and 1 cycle of 68° C. 10 min. The PCR product was assessed for quantity and size ranges with the 2100 Bioanalyzer (Agilent) using a DNA 7500 Chip (FIG. 1B). The PCR product was submitted to a second round of amplification in the same reaction solution as above. Amplification conditions consisted of: 1 cycle of 95° C., 10 min; 20 cycles of 95° C. 30 sec; −0.5° C./cycle 60° C. 30 sec; 68° C. 30 sec; 30 cycles of 95° C. 30 sec; 50° C. 30 sec; 68° C. 30 sec; and 1 cycle of 68° C. 10 min. The second PCR product was assessed for quantity and size ranges with the 2100 Bioanalyzer (Agilent) using a DNA 7500 Chip (FIG. 1C). The second PCR product was submitted to a third round of amplification in the same reaction solution as above. Amplification conditions consisted of: 1 cycle of 95° C., 10 min; 20 cycles of 95° C. 30 sec; −0.5° C./cycle 60° C. 30 sec; 68° C. 30 sec; 30 cycles of 95° C. 30 sec; 50° C. 30 sec; 68° C. 30 sec; and 1 cycle of 68° C. 10 min. The third PCR product was assessed for quantity and size ranges with the 2100 Bioanalyzer (Agilent) using a DNA 7500 Chip (FIG. 1D).

The small RNA cDNA library third PCR product was subcloned using the TOPO TA Cloning Kit (Invitrogen), and analyzed by Sanger sequencing. The sequences are shown in SEQ ID NOS. 7-14. The distribution of sequences, organized by origin, is shown in TABLE 1.

### Example 2

In one embodiment, plasma was isolated from a normal control (subject 2). Plasma was filtered through a 0.8 μm filter and divided into 1 mL aliquots. Aliquots were frozen at −80° C. until needed.

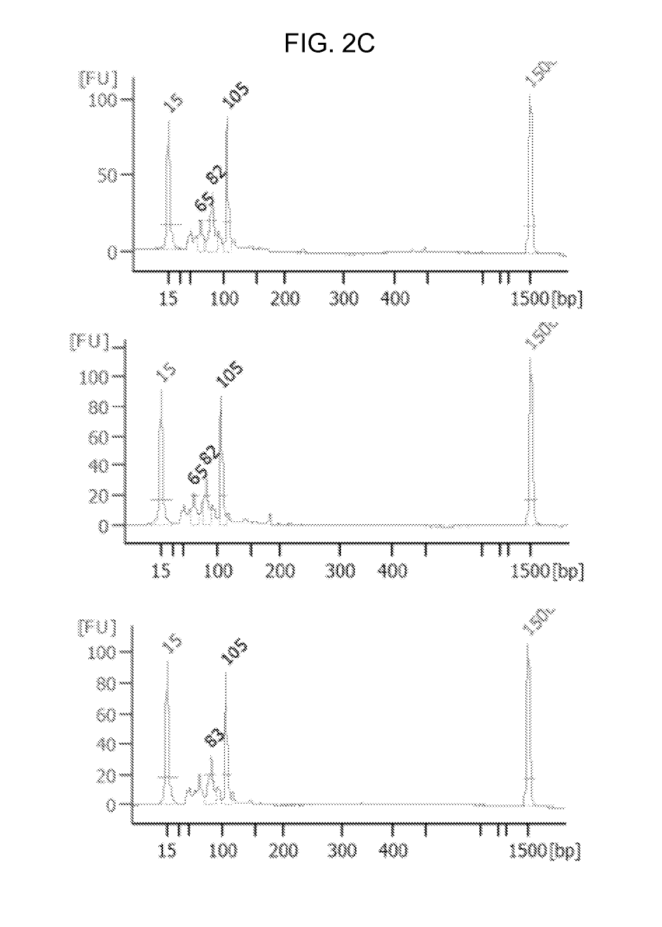

Isolation of microvesicle RNA was conducted using eight 1 mL aliquots of subject 1 plasma. The plasma was evenly split into four 2 mL aliquots, transferred to 5 mL polyallomer tubes (Beckman-Coulter) containing 8 μL RNasin Plus (40 u/μl, Promega) RNase inhibitor, and incubated for 5 min at room temp. Following incubation, the plasma aliquots were diluted in 3 mL PBS. Microvesicles were pelleted by ultracentrifugation at 120,000 g for 80 min. The microvesicle pellets were each washed in 42 μL PBS and 8 μL RNasin Plus, and incubated for 20 min at room temp. Microvesicle pellets were lysed in 700 ul Qiazol Reagent (Qiagen, Valencia, Ca., USA) and isolated using the miRNeasy kit (Qiagen). Each RNA aliquot was assessed for quality and concentration with the 2100 Bioanalyzer (Agilent) using a RNA 6000 Pico Chip (FIG. 2A)

Small RNA cDNA library preparation was performed as previously described (Pak) with modifications. Total microvesicle RNA was ligated at 37° C. for 1 hr and 16° C. for 16 hrs to a 5′-adenylated 3′-adaptor oligonucleotide (5′-rAppCTGTAGGCACCATCAAT/ddC/-3′ (SEQ ID NO: 1) (IDT DNA)) at a 1:100 molar ratio in a reaction volume of 80 μl containing 40 U T4 RNA Ligase I (New England BioLabs, Beverly, Ma., USA), 80 U RNasin Plus (Promega), 1.3% DMSO, and 1×T4 RNA Ligase Buffer (New England BioLabs). After ligation, the product was purified by phenol-chloroform extraction, filtered using a 30 kDa centrifugal filter unit (Millipore) to remove the 3′-adaptor oligonucleotide, and concentrated to 6 μL in a Speed Vac concentrator (Savant). The ligation product was reverse transcribed using Sensiscript (Qiagen) and a primer with sequence complementarity to the 3′-adaptor oligonucleotide (5′-GATTGATGGTGCCTACAG-3′ (SEQ ID NO: 2) (IDT DNA)). Following reverse transcription, the cDNA was purified by phenol-chloroform extraction, filtered using a 30 kDa centrifugal filter unit (Millipore) to remove the primer, and concentrated to 11 μL in a Speed Vac concentrator (Savant). The cDNA was ligated at 22° C. for 16 hrs to a second 5′-adenylated 3′-adaptor oligonucleotide (5′-rAppCACTCGGGCACCAAGGA/ddC/-3′ (SEQ ID NO: 3) (IDT DNA)) at a 1:30 molar ratio in a reaction volume of 20 μl containing 10 U T4 RNA Ligase I (New England BioLabs), 5% DMSO, 25% PEG8000, and 1×T4 RNA Ligase Buffer (New England BioLabs). After ligation, the cDNA was purified by phenol-chloroform extraction and filtered using a 30 kDa centrifugal filter unit (Millipore) to remove the second 5′-adenylated 3′-adaptor oligonucleotide.

The final ligation product was PCR amplified using the reverse transcription primer and a primer with sequence complementarity to the second 3′-adaptor (5′-GTCCTTGGTGCCCGAGTG-3′ (SEQ ID NO: 4) (IDT DNA)) in a reaction volume of 20 μL containing 1 U Platinum Taq DNA Polymerase (Invitrogen, Carlsbad, Ca., USA), 1× Platinum Taq DNA Polymerase Buffer, 3 mM MgCl₂, 0.5 mM dNTPs, and 0.5 μM of each primer. Amplification conditions consisted of: 1 cycle of 95° C., 10 min; 40 cycles of 95° C. 30 sec; 48° C. or 50° C. or 52° C. or 54° C. 30 sec; 68° C. 30 sec; and 1 cycle of 68° C. 10 min. The PCR products were assessed for quantity and size ranges with the 2100 Bioanalyzer (Agilent) using a DNA 7500 Chip (FIG. 2B). The PCR products were submitted to a second round of amplification in the same reaction solution as above. Amplification conditions consisted of: 1 cycle of 95° C., 10 min; 20 cycles of 95° C. 30 sec; −0.5° C./cycle 60° C. 30 sec; 68° C. 30 sec; 30 cycles of 95° C. 30 sec; 50° C. 30 sec; 68° C. 30 sec; and 1 cycle of 68° C. 10 min. The second PCR products were assessed for quantity and size ranges with the 2100 Bioanalyzer (Agilent) using a DNA 7500 Chip (FIG. 2C).

The small RNA cDNA library second PCR products were subcloned using the TOPO TA Cloning Kit (Invitrogen), according to the manufacturer's recommendations, and analyzed by Sanger sequencing. The sequences are shown in SEQ ID NOS. 15-130. The distribution of sequences, organized by origin, is shown in TABLE 2.

### Example 3

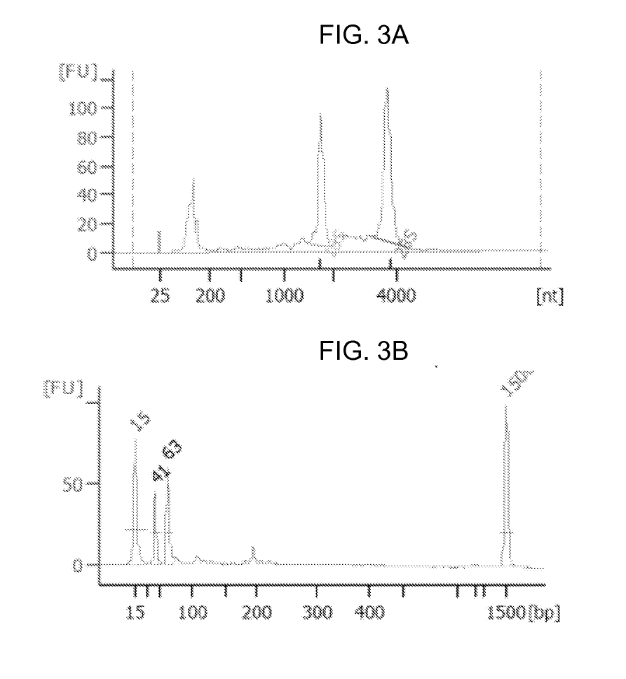

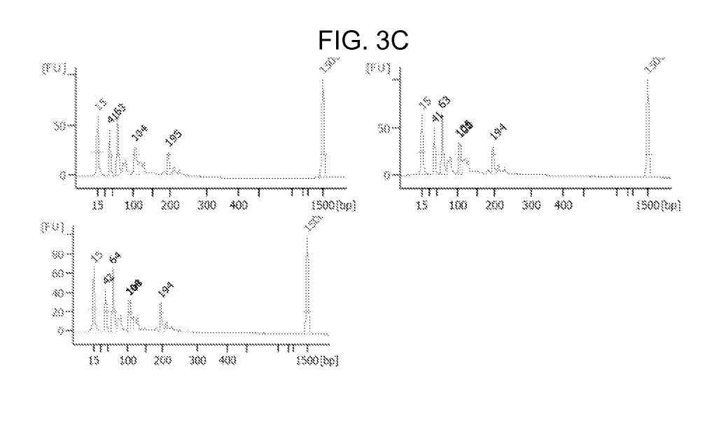

In one embodiment, leukocytes were isolated from a normal control (subject 2) and divided into two 1 mL aliquots. Leukocyte cells were lysed in 700 ul Qiazol Reagent (Qiagen, Valencia, Ca., USA) and isolated using the miRNeasy kit (Qiagen). The total RNA was quantified using a nanodrop ND-2000 instrument (Thermo Fischer Scientific, Wilmington, De., USA). Subject 1 leukocytes were found to contain ˜4 μg RNA/mL plasma (FIG. 1A). Each RNA aliquot was diluted to 5 ng/μl and assessed for quality with the 2100 Bioanalyzer (Agilent) using a RNA 6000 Pico Chip (FIG. 3A).

Small RNA cDNA library preparation was performed as previously described (Pak) with modifications. Total microvesicle RNA was ligated at 37° C. for 1 hr and 16° C. for 2 hrs to a 5′-adenylated 3′-adaptor oligonucleotide (5′-rAppCTGTAGGCACCATCAAT/ddC/-3′ (SEQ ID NO: 1) (IDT DNA)) at a 1:0.5 molar ratio in a reaction volume of 100 μl containing 50 U T4 RNA Ligase I (New England BioLabs, Beverly, Ma., USA), 100 U RNasin Plus (Promega), 5% DMSO, and 1×T4 RNA Ligase Buffer (New England BioLabs). After ligation, the product was purified by phenol-chloroform extraction and filtered using a 30 kDa centrifugal filter unit (Millipore) to remove the 3′-adaptor oligonucleotide. The ligation product was reverse transcribed using Omniscript (Qiagen) and a primer with sequence complementarity to the 3′-adaptor oligonucleotide (5′-GATTGATGGTGCCTACAG-3′ (SEQ ID NO: 2) (IDT DNA)), according to the manufacturer's recommendation. Following reverse transcription, the cDNA was purified by phenol-chloroform extraction, filtered using a 30 kDa centrifugal filter unit (Millipore) to remove the primer, and concentrated to 7 μL in a Speed Vac concentrator (Savant). The cDNA was ligated at 22° C. for 2 hrs to a second 5′-adenylated 3′-adaptor oligonucleotide (5′-rAppCACTCGGGCACCAAGGA/ddC/-3′ (SEQ ID NO: 3) (IDT DNA)) at a 1:3 molar ratio in a reaction volume of 20 μl containing 10 U T4 RNA Ligase I (New England BioLabs), 5% DMSO, 25% PEG8000, and 1×T4 RNA Ligase Buffer (New England BioLabs). After ligation, the cDNA was purified by phenol-chloroform extraction and filtered using a 30 kDa centrifugal filter unit (Millipore) to remove the second 5′-adenylated 3′-adaptor oligonucleotide.

The final ligation product was PCR amplified using the reverse transcription primer and a primer with sequence complementarity to the second 3′-adaptor (5′-GTCCTTGGTGCCCGAGTG-3′ (SEQ ID NO: 4) (IDT DNA)) in a reaction volume of 20 μL containing 1 U Platinum Taq DNA Polymerase (Invitrogen, Carlsbad, Ca., USA), 1× Platinum Taq DNA Polymerase Buffer, 3 mM MgCl₂, 0.5 mM dNTPs, and 0.5 μM of each primer. Amplification conditions consisted of: 1 cycle of 95° C., 10 min; 20 cycles of 95° C. 30 sec; −0.5° C./cycle 60° C. 30 sec; 68° C. 30 sec; 30 cycles of 95° C. 30 s; 50° C. 30 sec; 68° C. 30 sec; and 1 cycle of 68° C. 10 min. The PCR product was assessed for quantity and size ranges with the 2100 Bioanalyzer (Agilent) using a DNA 7500 Chip (FIG. 3B). The PCR product was submitted to a second round of amplification using PCR product template dilutions (no dilution; 1:1; and 1:4) in the same reaction solution and amplification conditions as above. The second PCR products were assessed for quantity and size ranges with the 2100 Bioanalyzer (Agilent) using a DNA 7500 Chip (FIG. 3C).

The small RNA cDNA library second PCR products were subcloned using the TOPO TA Cloning Kit (Invitrogen), and analyzed by Sanger sequencing. The sequences are shown in SEQ ID NOS. 131-221. The distribution of sequences, organized by origin, is shown in TABLE 3.

### Example 4

In one embodiment, normal control serum was isolated (subject 1 and 2). In another embodiment, we obtained normal control serum from a bioreclamation bank (subject 7). In another embodiment, we obtained serum from glioblastoma multiforme patients (subject 4-6). Serum was filtered through a 0.8 μm filter and divided into 1 mL aliquots. Aliquots were frozen at −80° C. until needed.

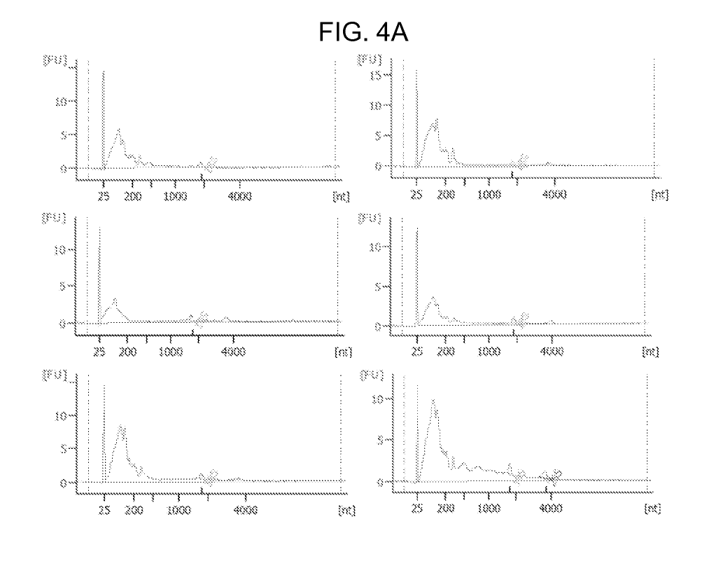

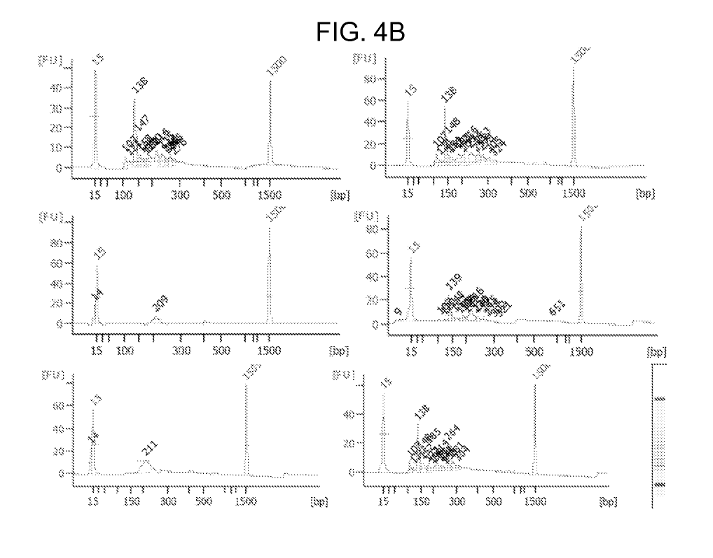

Isolation of microvesicle RNA was conducted using eight 1 mL aliquots of serum from each subject. For each subject, the serum was split into four 2 mL aliquots, transferred to 5 mL polyallomer tubes (Beckman-Coulter, Miami, Fl., USA) containing 8 μL RNasin Plus (40 U/μl, Promega, Madison, Wi., USA) RNase inhibitor, and incubated for 5 minutes at room temp. Following incubation, the plasma aliquots were diluted in 3 mL PBS. Microvesicles were pelleted by ultracentrifugation at 120,000 g for 80 minutes. The microvesicle pellets were each washed in 42 μL PBS and 8 μL RNasin Plus, and incubated for 20 minutes at room temp. Microvesicle pellets were lysed in 700 ul Qiazol Reagent (Qiagen, Valencia, Ca., USA) and isolated using the miRNeasy kit (Qiagen) according to the manufacturer's recommendation. For each subject aliquot, the RNA quality and concentration was assessed with the 2100 Bioanalyzer (Agilent, Palo Alto, Ca., USA) using a RNA 6000 Pico Chip. A representative subject profile is shown (FIG. 4A).

All four aliquots of total RNA from each subject were combined, purified by phenol-chloroform extraction, and concentrated using a 30 kDa centrifugal filter unit (Millipore, Bedford, Ma., USA). The total RNAs were further concentrated to ˜7 μL in a Speed Vac concentrator (Savant).

Small RNA cDNA library preparation was performed as previously described (Pak) with modifications. Total microvesicle RNAs were ligated at 16° C. for 16 hours to a 5′-adenylated 3′-adaptor oligonucleotide (5′-rAppTGGAATTCTCGGGCACCAAG/ddC/-3′ (SEQ ID NO: 5) (IDT DNA)) at a 1:100 molar ratio in a reaction volume of 20 μl containing 10 U T4 RNA Ligase I (10 U/μL; New England BioLabs, Beverly, Ma., USA), 20 U RNasin Plus (Promega), 10% DMSO, 12% PEG8000, and 1×T4 RNA Ligase Buffer (New England BioLabs). After ligation, the products were purified by phenol-chloroform extraction, filtered using a 30 kDa centrifugal filter unit (Millipore) to remove the 3′-adaptor oligonucleotide, and concentrated to 13 μL in a Speed Vac concentrator (Savant). The ligation products were reverse transcribed using Sensiscript (Qiagen) and a primer with sequence complementarity to the 3′-adaptor oligonucleotide (5′-GCTTGGTGCCCGAGAATTCCA-3′ (SEQ ID NO: 6) (IDT DNA)). Following reverse transcription, the cDNAs were purified by phenol-chloroform extraction, filtered using a 30 kDa centrifugal filter unit (Millipore) to remove the primer, and concentrated to ˜7 μL in a Speed Vac concentrator (Savant). The cDNAs were ligated at 16° C. for 16 hours to a second 5′-adenylated 3′-adaptor oligonucleotide (5′-rAppCACTCGGGCACCAAGGA/ddC/-3′ (SEQ ID NO: 3) (IDT DNA)) at a 1:100 molar ratio in a reaction volume of 20 μl containing 10 U T4 RNA Ligase I (New England BioLabs), 10% DMSO, 12% PEG8000, and 1×T4 RNA Ligase Buffer (New England BioLabs). After ligation, the cDNAs were purified by phenol-chloroform extraction and filtered using a 30 kDa centrifugal filter unit (Millipore) to remove the second 5′-adenylated 3′-adaptor oligonucleotide.

The final ligation products were PCR amplified using Phusion II (Thermo Fischer), and the reverse transcription primer and a primer with sequence complementarity to the second 3′-adaptor (5′-GTCCTTGGTGCCCGAGTG-3′ (SEQ ID NO: 4) (IDT DNA)). Amplification conditions consisted of: 1 cycle of 98° C., 30 sec; 35 cycles of 98° C. 10 sec; 67.4° C. 10 sec; 72° C. 30 sec; and 1 cycle of 72° C. 10 min. The PCR products were assessed for quantity and size ranges with the 2100 Bioanalyzer (Agilent) using a DNA 7500 Chip (FIG. 4B).

The small RNA cDNA library PCR products were subcloned using Zero Blunt Cloning Kit (Invitrogen), according to the manufacturer's recommendations, and analyzed by Sanger sequencing. The sequences are shown as follows: subject 7 in SEQ ID NOS. 222-236, subject 1 in SEQ ID NOS. 237-252, subject 2 in SEQ ID NOS. 253-268, subject 4 in SEQ ID NOS. 269-284, subject 5 in SEQ ID NOS. 285-300, and subject 6 in SEQ ID NOS. 301-313. The distribution of sequences, organized by origin, is shown in TABLE 4.

### Example 5

In one embodiment, normal control serum was isolated (subject 1,2). In another embodiment, normal control serum was obtained from a bioreclamation bank (subject 7). In another embodiment, serum was obtained from glioblastoma multiforme patients (subject 4-6). Serum was filtered through a 0.8 μm filter and divided into 1 mL aliquots. Aliquots were frozen at −80° C. until needed.

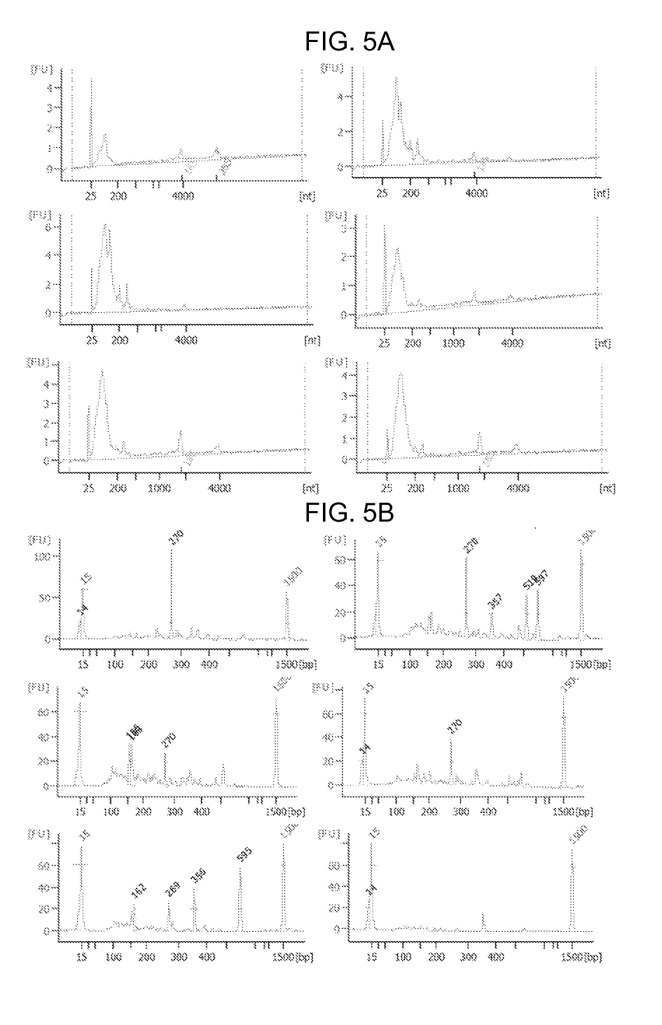

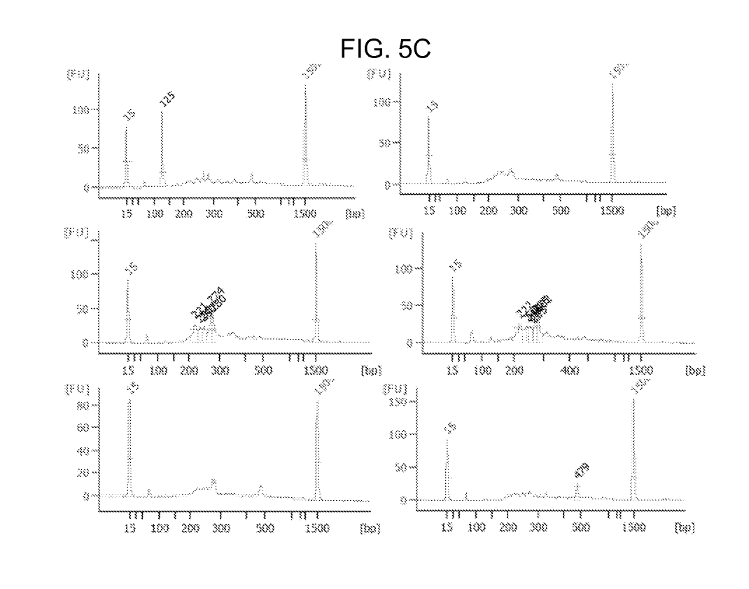

Isolation of microvesicle RNA was conducted using eight 1 mL aliquots of serum from each subject. For each subject, the serum was split into four 2 mL aliquots, transferred to 5 mL polyallomer tubes (Beckman-Coulter, Miami, Fl., USA) containing 8 μL RNasin Plus (40 U/μl, Promega, Madison, Wi., USA) RNase inhibitor, and incubated for 5 min at room temp. Following incubation, the plasma aliquots were diluted in 3 mL PBS. Microvesicles were pelleted by ultracentrifugation at 120,000 g for 80 minutes. The microvesicle pellets were each washed in 42 μL PBS and 8 μL RNasin Plus, and incubated for 20 minutes at room temp. For each subject, the four microvesicle pellets were lysed in 1.4 mL Qiazol Reagent (Qiagen, Valencia, Ca., USA) and isolated using the miRNeasy kit (Qiagen). Total RNAs were then treated for 20 minutes at room temp with 2 U of DNase I (DNA free kit, Ambion). After treatment, the DNase I was inactivated using the kit's inactivation reagent. The RNA qualities and concentrations were assessed with the 2100 Bioanalyzer (Agilent) using a RNA 6000 Pico Chip (FIG. 5A).

Small RNA cDNA library preparation was performed as previously described (Pak) with a few modifications. Total microvesicle RNAs were ligated at 16° C. for 16 hours to a 5′-adenylated 3′-adaptor oligonucleotide (5′-rAppTGGAATTCTCGGGCACCAAG/3ddC/-3′ (SEQ ID NO: 5) (IDT DNA)) at a 1:100 molar ratio in a reaction volume of 30 μl containing 15 U T4 RNA Ligase I (New England BioLabs), 30 U RNasin Plus (Promega), 10% DMSO, 12% PEG 8000, and 1×T4 RNA Ligase Buffer (New England BioLabs). After ligation, the products were purified and concentrated with a 30 kDa centrifugal filter unit (Millipore). The ligated products were then reverse transcribed using Sensiscript (Qiagen) and a primer with sequence complementarity to the 3′-adaptor (5′-GCTTGGTGCCCGAGAATTCCA-3′ (SEQ ID NO: 6) (IDT DNA)), according to the manufacturer's recommendation. The cDNAs were purified and concentrated with a 30 kDa centrifugal filter unit (Millipore). The cDNAs were ligated at 16° C. for 16 hours to a second 5′-adenylated 3′-adaptor oligonucleotide (5′-rAppCACTCGGGCACCAAGGA/3ddC/-3′ (SEQ ID NO: 3) (IDT DNA)) at a 1:100 molar ratio in a reaction volume of 60 μl containing 15 U T4 RNA Ligase I (New England BioLabs), 10% DMSO, 12% PEG 8000, and 1×T4 RNA Ligase Buffer (New England BioLabs). The ligation products were purified and concentrated with a 30 kDa centrifugal filter unit (Millipore).

The final ligation products were PCR amplified using Phusion II (Thermo Fischer) and the reverse transcription primer and a primer with sequence complementarity to the second 3′-adaptor (5′-GTCCTTGGTGCCCGAGTG-3′ (SEQ ID NO: 4) (IDT DNA)). Amplification conditions consisted of: 1 cycle of 98° C., 30 sec; 35 cycles of 98° C. 10 sec; 67.4° C. 10 sec; 72° C. 30 sec; and 1 cycle of 72° C. 10 min. The PCR products were assessed for quantity and size ranges with the 2100 Bioanalyzer (Agilent) using a DNA 7500 Chip (FIG. 5B).

In one embodiment, the small RNA cDNA library PCR products were subcloned using Zero Blunt Cloning Kit (Invitrogen), and analyzed by Sanger sequencing. The sequences are shown as follows: subject 3 in SEQ ID NOS. 314-327, subject 1 in SEQ ID NOS. 328-353, subject 2 in SEQ ID NOS. 354-379, subject 4 in SEQ ID NOS. 380-402, subject 5 in SEQ ID NOS. 403-430, and subject 6 in SEQ ID NOS. 431-460. The distribution of sequences, organized by origin, is shown in TABLE 5.

In another embodiment, the small RNA cDNA library PCR products were prepared for Illumina sequencing. Library preparation was based on the manufacturer's recommendations (Illumina). Briefly, the small RNA cDNA library PCR products were purified using the QIAquick PCR Purification kit (Qiagen). The products were phosphorylated with 50 U T4 Polynucleotide Kinase (New England BioLabs) in 1×T4 DNA Ligase Buffer (New England BioLabs) and purified using the MinElute Reaction Cleanup Kit (Qiagen). The products were A-tailed using NEBNext dA-Tailing Module and purified using the MinElute Reaction Cleanup Kit (Qiagen). The products were ligated to Illumina paired-end adaptor oligonucleotides and purified using the MinElute Reaction Cleanup Kit (Qiagen). The products were enriched by PCR using Phusion II (Thermo Fischer) for 25 cycles with Illumina PCR primer PE 1.0 and 2.0 and a PCR index primer (Subject 3: Index 7; Subject 1: Index 8; Subject 2: Index 9; Subject 4: Index 10; Subject 5: Index 11; Subject 6: Index 12). The PCR products were purified with the QIAquick PCR Purification kit (Qiagen). The PCR products were assessed for quantity and size range with the 2100 Bioanalyzer using a DNA 7500 Chip (Agilent) (FIG. 5C). The amplicons were sequenced with 150-bp paired-end reads on an Illumina MiSeq instrument. A summary of relevant HERV and tRNA sequences is shown TABLE 6.

TABLE 1
Classification of microvesicle RNA sequencing reads
for normal control (subject 1) plasma. Values are
as a percentage of total sequencing reads.

| Category | Percentage |
|---|---|
| Chromosomal tRNA | 50 |
| Alanine | 25 |
| Glycine | 25 |
| Unknown Origin | 37.5 |
| Mitochondrial tRNA | 12.5 |
| Leucine | 12.5 |

TABLE 2
Classification of microvesicle RNA sequencing reads
for normal control (subject 2) plasma. Values are
as a percentage of total sequencing reads.

| Category | Percentage |
|---|---|
| Mitochondrial tRNA | 52.6 |
| Threonine | 14.7 |
| Valine | 11.2 |
| Glycine | 6.0 |
| Tryptophan | 4.3 |
| Isoleucine | 3.4 |
| Leucine | 3.4 |
| Serine | 3.4 |
| Histidine | 2.6 |
| Lysine | 1.7 |
| Arginine | 0.9 |
| Glutamate | 0.9 |
| Methionine | 0.9 |
| Chromosomal tRNA | 28.4 |
| Glycine | 8.6 |
| Alanine | 5.2 |
| Arginine | 4.3 |
| Proline | 3.4 |
| Valine | 3.4 |
| Cysteine | 1.7 |
| Serine | 0.9 |
| Threonine | 0.9 |
| Unknown Origin | 12.1 |
| RNY RNA | 4.3 |
| Mitochondrial rRNA | 0.9 |
| 16S | 0.9 |
| microRNA | 0.9 |

TABLE 3
Classification of cellular RNA sequencing reads
for normal control (subject 1) leukocytes. Values
are as a percentage of total sequencing reads.

| Category | Percentage |
|---|---|
| Chromosomal tRNA | 53.8 |
| Proline | 14.3 |
| Glycine | 9.9 |
| Alanine | 5.5 |
| Aspartate | 5.5 |
| Arginine | 4.4 |
| Leucine | 3.3 |
| Valine | 3.3 |
| Glutamate | 2.2 |
| Cysteine | 1.1 |
| Methionine | 1.1 |
| Chromosomal rRNA | 20.9 |
| 28S | 17.6 |
| 5S | 2.2 |
| 18S | 1.1 |
| Mitochondrial tRNA | 9.9 |
| Serine | 4.4 |
| Valine | 4.4 |
| Lysine | 1.1 |
| 7SL RNA | 4.4 |
| Mitochondrial rRNA | 4.4 |
| 16S | 4.4 |
| RNY RNA | 3.3 |
| Chromosomal mRNA | 1.1 |
| snoRNA | 1.1 |
| Unknown Origin | 1.1 |

TABLE 5
Classification of RNA sequencing reads for normal control (subjects 1-3) and gliblastoma multiforme
(subjects 4-6) serum. Values are as a percentage of total sequencing reads. Chr, chromosomal;
Mt, mitochondrial; Val, valine; Asp, aspartate;

| | | Percentage | | | | | | | | | | |
|---|---|---|---|---|---|---|---|---|---|---|---|---|
| | | Chr rRNA | | | Mt rRNA | Mt tRNA | | | | | | |
| | | 28S | 18S | 5S | 16S | Asn | Trp | Val | Chr mRNA | Rep Element | 7SL RNA | Ukn Function |
| Normal | Subject 7 | 0 | 0 | 6.7 | 0 | 0 | 0 | 0 | 0 | 86.7 | 0.0 | 6.7 |
| Controls | Subject 1 | 50.0 | 6.3 | 0 | 18.8 | 0 | 0 | 0 | 6.3 | 0 | 0 | 12.5 |
| | Subject 2 | 56.3 | 0 | 0 | 25.0 | 6.3 | 6.3 | 0 | 0 | 0 | 6.3 | 0 |
| Glioblastoma | Subject 4 | 68.8 | 0 | 0 | 18.8 | 0 | 0 | 6.3 | 0 | 0 | 6.3 | 0 |
| Multiformes | Subject 5 | 68.8 | 0 | 0 | 12.5 | 0 | 0 | 6.3 | 0 | 0 | 6.3 | 6.3 |
| | Subject 6 | 15.4 | 0 | 0 | 0 | 0 | 0 | 0 | 0 | 46.2 | 0.0 | 38.5 |

TABLE 4
Classification of microvesicle RNA sequencing reads for normal control (subject 1, 2, 7) and gliblastoma multiforme (subjects 4-6) serum.
Values are as a percentage of total sequencing reads. It is thought that the sequencing reads from subjects 6 and 7 represent genomic DNA.

| | | Percentage | | | | | | | | | | | |
|---|---|---|---|---|---|---|---|---|---|---|---|---|---|
| | | Chr rRNA | | | Mt tRNA | | | | Mt rRNA | | | | |
| | | 28S | 18S | 5S | Val | Ile | Asp | Ser | 16S | Chr mRNA | Ukn Origin | Ukn Function | Bac Origin |
| Normal Controls | Subject 1 | 46.2 | 34.6 | 0 | 15.4 | 0 | 0 | 0 | 0 | 0 | 3.8 | 0 | 0 |
| | Subject 2 | 50.0 | 7.7 | 0 | 7.7 | 3.8 | 0 | 0 | 23.1 | 7.7 | 0 | 0 | 0 |
| | Subject 3 | 42.9 | 0 | 0 | 4.3 | 0 | 4.3 | 4.3 | 0 | 21.4 | 14.3 | 14.3 | 7.1 |
| Glioblastoma | Subject 4 | 34.8 | 4.3 | 0 | 0 | 0 | 0 | 0 | 30.4 | 13.0 | 0 | 0 | 4.3 |
| Multiformes | Subject 5 | 53.6 | 25.0 | 3.6 | 0 | 0 | 0 | 0 | 7.1 | 0 | 0 | 3.6 | 7.1 |
| | Subject 6 | 16.7 | 20.0 | 0 | 3.3 | 3.3 | 0 | 0 | 0 | 10.0 | 33.3 | 10.0 | 3.3 |

Chr, chromosomal; Mt, mitochondrial; Asn, asparagine; Trp, tryptophan; Val, valine; Rep, repetitive; Ukn, Unknown.

TABLE 6
Classification of HERVH and tRNA-His sequencing reads for normal
control (subjects 1-3) and glioblastoma multiforme (subjects 4-6) serum.
Values are as a normalization of total sequencing reads.

| | HERVH | tRNA-His |
|---|---|---|
| Subject 1 | 6 | 0 |
| Subject 2 | 0 | 0 |
| Subject 3 | 4 | 0 |
| Subject 4 | 110 | 3 |
| Subject 5 | 2 | 0 |
| Subject 6 | 0 | 0 |

### Sequences

### Example 1

### Subject 1

SEQ ID NO: 7
CCCCAAAAATTTTGGTGCAACTCCAAATAAAAGTACCA
mitochondrial tRNA Leucine
SEQ ID NO: 8
TCCCCGGCCTNTNNNCCA chromosomal tRNA Glycine
SEQ ID NO: 9
TTCCCGGCCAATGCACCA chromosomal tRNA Glycine
SEQ ID NO: 10
TCCCCGGCATCTCCACCA chromosomal tRNA Alanine
SEQ ID NO: 11
GCTAAAGGGGGCAGA Unknown Origin
SEQ ID NO: 12
NANAGCGAGNCNNNNNNNNNNANNAACTGNNANAACACTTCCCGGCCANC
AAANNNNTTNNCTGGTGTGATC Unknown Origin
SEQ ID NO: 13
TCCCCAGCATCTCCACCA chromosomal tRNA Alanine
SEQ ID NO: 14
GCTAAAGGGGGCAGA Unknown Origin

### Example 2

### Subject 2

SEQ ID NO: 15
CGCA Unknown Origin
SEQ ID NO: 16
ATCCGGGTGCCCCCTCCA chromosomal tRNA Cysteine
SEQ ID NO: 17
TATAAATAGTACCGTTAACTTCCAATTAACTAGTTTTGACAACATTCAAA
AAAGAGTACCA mitochondrial tRNA Glycine
SEQ ID NO: 18
TTCCCGGCCAATGCACCA
chromosomal tRNA Glycine (SEQ ID NO: 11)
SEQ ID NO: 19
TTCCCGGCCAATGCACCA
chromosomal tRNA Glycine (SEQ ID NO: 11)
SEQ ID NO: 20
CACCTCTTTACAGTGACCA mitochondrial tRNA Lysine
SEQ ID NO: 21
AACCGGAGATGAAAACCTTTTTCCAAGGACACCA mitochondrial
tRNA Threonine
SEQ ID NO: 22
TTGTAAACCGGAGATGAAAACCTTTTTCCAAGGACACCA
mitochondrial tRNA Threonine
SEQ ID NO: 23
TTGTAAACCGGAGATGAAAACCTTTTTCCAAGGACACCA
mitochondrial tRNA Threonine
SEQ ID NO: 24
GNGTAAATAATAGGAGNTTAAACNNNNNNNNTTNTNCCA
mitochondrial tRNA Isoleucine
SEQ ID NO: 25
CTTAACACAAAGCACCCAACTTACACTTAGGAGATTTCAACTTAACTTGA
CCGCTCTGACCA mitochondrial tRNA Valine
SEQ ID NO: 26
TATAAATAGTACCGTTAACTTCCAATTAACTAGTTTTGACAACATTCAAA
AAAGAGTACCA mitochondrial tRNA Glycine
SEQ ID NO: 27
TATAAATAGTACCGTTAACTTCCAATTAACTAGTTTTGACAACATTCAAA
AAAGAGTACCA mitochondrial tRNA Glycine
SEQ ID NO: 28
CTTAACACAAAGCACCCAACTTACACTTAGGAGATTTCAACTTAACTTGA
CCGCTCTGACCA mitochondrial tRNA Valine
SEQ ID NO: 29
CTTAACACAAAGCACCCAACTTACACTTAGGAGATTTCAACTTAACTTGA
CCGCTCTGACCA mitochondrial tRNA Valine
SEQ ID NO: 30
TTCCCGGCCAATGCACCA
chromosomal tRNA Glycine (SEQ ID NO: 11)
SEQ ID NO: 31
TTCCCGGCCCATGCACCA chromosomal tRNA Glycine
SEQ ID NO: 32
TATAAATAGTACCGTTAACTTCCAATTAACTAGTTTTGACAACATTCAAA
AAAGAGTACCA mitochondrial tRNA Glycine
SEQ ID NO: 33
ATCCCACCAGAGTCCCCA chromosomal tRNA Arginine
SEQ ID NO: 34
TTCCCGNCCAANNCNCCA chromosomal tRNA Glycine
SEQ ID NO: 35
AACACAAAGCACCCAACTTACACTTAGGAGATTTCAACTTAACTTGACCG
CTCTGACCA mitochondrial tRNA Valine
SEQ ID NO: 36
CCCCCCTTATTTCTACCA mitochondrial tRNA Isoleucine
SEQ ID NO: 37
TTGTAAACCGGAGATGAAAACCTTTTTCCAAGGACACCA
mitochondrial tRNA Threonine
SEQ ID NO: 38
GACAACAGAGGCTTACGACCCCTTATTTACCCCA
mitochondrial tRNA Histidine
SEQ ID NO: 39
GAGAAAGCTCACAAGAACTGCTAACTCATGCCCCCATGTCTAACAACATG
GCTTTCTCACCA mitochondrial tRNA Serine
SEQ ID NO: 40
GAGTAAATAATAGGAGCTTAAACCCCCTTATTTCTACC
mitochondrial tRNA Isoleucine
SEQ ID NO: 41
CTTAACACAAAGCACCCAACTTACACTTAGGAGATTTCAACTTAACTTGA
CCGCTCTGACCA mitochondrial tRNA Valine
SEQ ID NO: 42
CTTAACACAAAGCACCCAACTTACACTTAGGAGATTTCAACTTAACTTGA
CCGCTCTGACCA mitochondrial tRNA Valine
SEQ ID NO: 43
CTTAACACAAAGCACCCAACTTACACTTAGGAGATTTCAACTTAACTTGA
CCGCTCTGACCA mitochondrial tRNA Valine
SEQ ID NO: 44
AGCCCTCAGTAAGTTGCAATACTTAATTT mitochondrial
tRNA Tryptophan
SEQ ID NO: 45
GAGCAGAACCCAACCTCCGAGCAGTACA mitochondrial
rRNA 16S
SEQ ID NO: 46
AACACAAAGCACCCAACTTACACTTAGGAGATTTCAACTTAACTTGACCG
CTCTGACCA mitochondrial tRNA Valine
SEQ ID NO: 47
GGCTGGTCCGATGGTAGTGGGTTATCAGAACA RNY4 RNA
SEQ ID NO: 48
CTTAACACAAAGCACCCAACTTACACTTAGGAGATTTCAACTTAACTTGA
CCGCTCTGACCA mitochondrial tRNA Valine
SEQ ID NO: 49
CTTAACACAAAGCACCCAACTTACACTTAGGAGATTTCAACTTAACTTGA
CCGCTCTGACCA mitochondrial tRNA Valine
SEQ ID NO: 50
ATCCCGGACGAGCCCCCA chromosomal tRNA Proline
SEQ ID NO: 51
GACAACAGAGGCTTACGACCCCTTATTTACCGCCA
mitochondrial tRNA Histidine
SEQ ID NO: 52
ATCCCAGTCT Unknown Origin
SEQ ID NO: 53
AGCATCTCCACCA chromosomal tRNA Alanine
SEQ ID NO: 54
CCGAAAGGCATGCCTGTTTGAGTGTCA Unknown Origin
SEQ ID NO: 55
AGTAAGGTCAGCTAAATAAGCTATCGGGCCCATACCCCGAAAATGTTGGT
TATACCCTTCCCGTACTACCA mitochondrial
tRNA Methionine
SEQ ID NO: 56
AACCGGAGATGAAAACCTTTTTCCAAGGACACCA
mitochondrial tRNA Threonine
SEQ ID NO: 57
GNNGNTNNGANNNTNGTGGGTTATCNNA RNY4 RNA
SEQ ID NO: 58
ATCCCGGACGAGCCCCCA chromosomal tRNA Proline
SEQ ID NO: 59
AGCATNTCCNCCA chromosomal tRNA Alanine
SEQ ID NO: 60
CTTAACACAAAGCACCCAACTTACACTTAGGAGATTTCAACTTAACTTGA
CCGCTCTGACCA mitochondrial tRNA Valine
SEQ ID NO: 61
AGTAAACCGGAGATGAAAACCTTTNTNCAAGGACACCA
mitochondrial tRNA Threonine
SEQ ID NO: 62
CTTAACACAAAGCACCCAACTTACACTTAGGAGATTTCAACTTAACTTGA
CCGCTCTGACCA mitochondrial tRNA Valine
SEQ ID NO: 63
G Unknown Origin
SEQ ID NO: 64
ATCCCATCCTNGTCGCCA chromosomal tRNA Serine
SEQ ID NO: 65
GATGAAAACCTTTTTCCAAGGACACCA
mitochondrial tRNA Threonine
SEQ ID NO: 66
AGCATCTCCACCA chromosomal tRNA Alanine
SEQ ID NO: 67
GGCTGGTCCGATGGTAGTGGGTTATCAGAACT RNY4 RNA
SEQ ID NO: 68
TTGTAAACCGGAGATGAAAACCTTTTTCCAAGGACACCA
mitochondrial tRNA Threonine
SEQ ID NO: 69
AGCCCTCAGTAAGTTGCAATACTTAATTTCTGCCA
mitochondrial tRNA Tryptophan
SEQ ID NO: 70
GACTCGGCGGAAACACCA Unknown Origin
SEQ ID NO: 71
GTCGTGGTTGTAGTCCGTGCGAGAATACCN
mitochondrial tRNA Glutamate
SEQ ID NO: 72
GACGAGCCCCCA chromosomal tRNA Proline
SEQ ID NO: 73
ACCCAAGAACAGGNNNNANAN Unknown Origin
SEQ ID NO: 74
TATAAACTAATACACCAG mitochondrial
tRNA Threonine
SEQ ID NO: 75
ANCCGGGCGGAAACACCA chromosomal tRNA Valine
SEQ ID NO: 76
GGCNGAAACACCN chromosomal tRNA Valine
SEQ ID NO: 77
GGAGATGAAAACCTTTTTCCAAGGACACCA
mitochondrial tRNA Threonine
SEQ ID NO: 78
G Unknown Origin
SEQ ID NO: 79
GGNGGNNNNNNNNN Unknown Origin
SEQ ID NO: 80
CTCCAAATAAAAGTACCA mitochondrial
tRNA Leucine
SEQ ID NO: 81
CCA Unknown Origin
SEQ ID NO: 82
TTGTAAACCGGAGATGAAAACCTTTTTCCAAGGACACCA
mitochondrial tRNA Threonine
SEQ ID NO: 83
TATAAATAGTACCGTTAACTTCCAATTAACTAGTTTTGACAACATTCAAA
AAAGAGTACCA mitochondrial tRNA Glycine
SEQ ID NO: 84
GGTNNNATTCCTTCCTTTTTTGCCN mitochondrial
tRNA Serine
SEQ ID NO: 85
GGCTGGTCCGATGGTAGTGGGTTATCAGAACT RNY4 RNA
SEQ ID NO: 86
TTCCTCTTNTTAACA mitochondrial tRNA Leucine
SEQ ID NO: 87
AATGATTTCGACTCATTAAATTATGATAATCATATTTACCAACCA
mitochondrial tRNA Arginine
SEQ ID NO: 88
TTCCTCTTCTTAACACCA mitochondrial tRNA
Leucine
SEQ ID NO: 89
TATAAATAGTACCGTTAACTTCCAATTAACTAGTTTTGACAACATTCAAA
AAAGAGTACCA mitochondrial tRNA Glycine
SEQ ID NO: 90
GNNNNCTCCACCA chromosomal tRNA Alanine
SEQ ID NO: 91
TATAAACTAATACACCAGTACCCA mitochondrial
tRNA Threonine
SEQ ID NO: 92
TTCCCGGCCAATGCACCA
chromosomal tRNA Glycine (SEQ ID NO: 11)
SEQ ID NO: 93
TTCCCGGCCAATGCACCA
chromosomal tRNA Glycine (SEQ ID NO: 11)
SEQ ID NO: 94
TTGTAAACCGGAGATGAAAACCTTTTTCCAAGGACACCA
mitochondrial tRNA Threonine
SEQ ID NO: 95
CCAC Unknown Origin
SEQ ID NO: 96
CTCCTGGCTGGCTCGCCA chromosomal
tRNA Arginine
SEQ ID NO: 97
AACCGGGCGGAAACACCA chromosomal
tRNA Valine
SEQ ID NO: 98
NNNNTNNNNNNNNTCNCCA chromosomal
tRNA Arginine
SEQ ID NO: 99
TCGTACCGTGAGTAATAATGCG microRNA 126
SEQ ID NO: 100
GAGTAAATAATAGGAGCTTAAACCCCCTTATTTCTACCN
mitochondrial tRNA Isoleucine
SEQ ID NO: 101
ATCCCGGACGAGCCCCCA chromosomal
tRNA Proline
SEQ ID NO: 102
TAATTTCTGCCA mitochondrial tRNA
Tryptophan
SEQ ID NO: 103
ATCNCGGTGNNNCCTCCA chromosomal tRNA
Cysteine
SEQ ID NO: 104
TGGCAGAAATTAAGTATTGCAACTTACTGAGGG
mitochondrial tRNA Tryptophan
SEQ ID NO: 105
CTTAACACAAAGCACCCAACTTACACTTAGGAGATTTCAACTTAACTTGA
CCGCTCTGACCA mitochondrial tRNA Valine
SEQ ID NO: 106
AACCGGAGATGAAAACCTTTTTCCAAGGACACCA
mitochondrial tRNA Threonine
SEQ ID NO: 107
TTCCCGGCCAATGCACCA chromosomal
tRNA Glycine
SEQ ID NO: 108
TTGTAAACCGGAGATGAAAACCTTTTTCCAAGGACACCA
mitochondrial tRNA Threonine
SEQ ID NO: 109
GTTTAACCAAAACATCAGATTGTGAATCTGACAACAGAGGCTTACGACCC
CTTATTTACCCCA mitochondrial tRNA Histidine
SEQ ID NO: 110
TAAGTTGCAATACTTAATTTCTGCCA mitochondrial
tRNA Tryptophan
SEQ ID NO: 111
GAGAAAGCTCACAAGAACTGCTAACTCATGCCCCCATGTCTAACAACATG
mitochondrial tRNA Serine
SEQ ID NO: 112
GCATNTCCACCA chromosomal tRNA Alanine
SEQ ID NO: 113
TTGTAAACCGGAGATGAAAACCTTTTTCCAAGGACACCA
mitochondrial tRNA Threonine
SEQ ID NO: 114
TTCCCGGCCAATGCACCA chromosomal tRNA Glycine
SEQ ID NO: 115
GACCTGCCGC Unknown Origin
SEQ ID NO: 116
AGCATCTCCACCA chromosomal tRNA Alanine
SEQ ID NO: 117
CTCCTGGCTGGCTCGCCA chromosomal tRNA Arginine
SEQ ID NO: 118
AACCGGGCGGAAACACCA chromosomal tRNA Valine
SEQ ID NO: 119
CTTCTTNNCCCNN mitochondrial tRNA Leucine
SEQ ID NO: 120
TTGTAAACCGGAGATGAAAACCTTTTTCCAAGGACACCA
mitochondrial tRNA Threonine
SEQ ID NO: 121
GCG Unknown Origin
SEQ ID NO: 122
ATCTCGCTGGGGCCTCCA chromosomal tRNA Threonine
SEQ ID NO: 123
CACTTCTGACNCC Unknown Origin
SEQ ID NO: 124
CTCCTGGCTGGCTCGCCA chromosomal tRNA Arginine
SEQ ID NO: 125
AAGTTAAAGATTAAGAGAACCAACACCTCTTTACAGTGACCA
mitochondrial tRNA Lysine
SEQ ID NO: 126
GAAAGCTCNNNNNNNNNGNNNNCTCANGCCNNNNNGTNNNNCAACATGNC
TTTCNNNNCA mitochondrial tRNA Serine
SEQ ID NO: 127
GACT Unknown Origin
SEQ ID NO: 128
NTCCNGGCCNNNNCNCCA chromosomal tRNA Glycine
SEQ ID NO: 129
TATAAATAGTACCGTTAACTTCCAATTAACTAGTTTTGACAACATTCAAA
AAAGAGTACCA mitochondrial tRNA Glycine
SEQ ID NO: 130
CTGGTCCGATGGTAGTGGGTTATCAGAACA RNY4 RNA

### Example 3

### Subject 1

SEQ ID NO: 131
AACCGGGCGGAAACNNCNchromosomaltRNA Valine
SEQ ID NO: 132
GGGTTTCGTACGTAGCAGAGCAGCTCCCTCGCTGCGATCTATTGAAAGTCAGCCCTCG
ACACAAGGGTTTGT chromosomal 28S rRNA
SEQ ID NO: 133
GTATAGTGGTGAGTATCCCCGCCTGTCACGCGGGAGACCGGGGTTCGATTCCCCGACG
GGGAGCCA chromosomal tRNA Aspartate
SEQ ID NO: 134
GCCGGTCCCCCA Unknown Origin
SEQ ID NO: 135
GCGATTTGTCTGGTTAATTCCGATAACGAACGAGACTCTGGCATGCTAACTAGTTACG
CGACCCC chromosomal 18S rRNA
SEQ ID NO: 136
TGGGTCGGGGTTTCGTACGTAGCAGAGCAGCTCCCTCGCTGCGATCTATTGAAAGTCA
GCCCTCGACACAAGGGTTTGT chromosomal 28S rRNA
SEQ ID NO: 137
GTCCCATCTGGGGTG(GCCTGTGACTTTT) similar to chromosomal tRNA
Arginine
SEQ ID NO: 138
CTCCTGGCTGGCTCGCCA chromosomal tRNA Arginine
SEQ ID NO: 139
GCAATAGATATAGTACCGCAAGGGAAAGATGAAAAATTATAACCAAGCATAATATAGC
AGGGACTAACCCCTATACCTTCTGCATAATGAATTAACTAGAAATAACTTTGCAAGGAGAGCCAAAGCTAA
GACCCCCGAAACCAGACGAGCTACCTAAGAACAGCTAA mitochondrial 16S rRNA
SEQ ID NO: 140
TTCCCGGCCAATGCACCA chromosomal tRNA Glycine
SEQ ID NO: 141
ATCCGGGTGCCCCCTCCA chromosomal tRNA Cysteine
SEQ ID NO: 142
GGGTCGGGGTTTCGTACGTAGCAGAGCAGCTCCCTCGCTGCGATCTATTGAAAGTCAG
CCCTCGACACAAGGGTTTGT chromosomal 28S rRNA
SEQ ID NO: 143
GCAATAGATATAGTACCGCAAGGGAAAGATGAAAAATTATAACCAAGCATAATATAGC
AGGGACTAACCCCTATACCTTCTGCATAATGAATTAACTAGAAATAACTTTGCAAGGAGAGCCAAAGCTAA
GACCCCCGAAACCAGACGAGCTACCTAAGAACAGCTAA mitochondrial 16S rRNA
SEQ ID NO: 144
ATCCCGGACGAGCCCCCA chromosomal tRNA Proline
SEQ ID NO: 145
TTCCCGGNCNNTGCACCA chromosomal tRNA Glycine
SEQ ID NO: 146
TTCCCGGCCAATGCACCA chromosomal tRNA Glycine
SEQ ID NO: 147
GTCTAGCGGTTAGGATTCCTGGTTTTCACCCAGGCGGCCCGGGTTCGACTCCCGGTGT
GGGAACCA chromosomal tRNA Glutamate
SEQ ID NO: 148
ATCCCGGCCGANCCCCCA chromosomal tRNA Proline
SEQ ID NO: 149
ATCCCGGACGAGCCCCCA chromosomal tRNA Proline
SEQ ID NO: 150
CTCCTGGCTGGCTCGCCA chromosomal tRNA Arginine
SEQ ID NO: 151
ACCTCAGAGGGGGCA(GCTGCCATTT) similar to chromosomal tRNA
Methionine
SEQ ID NO: 152
ATCCCGGACGAGCCCCCA chromosomal tRNA Proline
SEQ ID NO: 153
AACCGGGCGGAAACACCA chromosomal tRNA Valine
SEQ ID NO: 154
AACCGGGCGGAAACACCA chromosomal tRNA Valine
SEQ ID NO: 155
GGCTGGTCCGATGGTAGTGGGTTATCAGAACTTATTAACATTAGTGTCACTAAAGTTG
GTATACAACCCCCCACTGCTAAATTTGACTGGCTT RNY4 RNA
SEQ ID NO: 156
NTCCCGGNCNNNNNNCCA chromosomal tRNA Proline
SEQ ID NO: 157
ATCCCGGACGANCCCCCA chromosomal tRNA Proline
SEQ ID NO: 158
TAGGCTTT chromosomal 5S rRNA
SEQ ID NO: 159
GGCTGGTCCGAGTGCAGTGGTGTTTACAACTAATTGATCACAACCAGTTACAGATTTC
TTTGTTCCTTCTCCACTCCCACTGCTTCACTTGACTAGT RNY3 RNA
SEQ ID NO: 160
ATCCCGGACGAGCCCCCA chromosomal tRNA Proline
SEQ ID NO: 161
GTCACGCACCGCACGTTCGTGGGGAACCTGGCGNNAANNCATTCGTAGACGACCTGCT
TCTGGGTCGGGGTTTCGTACGTAGCAGAGCAGCTCCCTCGCTGCGATCTATTGAAAGTCAGCCCTCGACAC
AAGGGTTTGT chromosomal 28S rRNA
SEQ ID NO: 162
ATCCCGGACGAGCCCCCA chromosomal tRNA Proline
SEQ ID NO: 163
TTCCCGGCCAATGCACCA chromosomal tRNA Glycine
SEQ ID NO: 164
GTCCCATCTGGGGTG(GCCTGTGACTTTT) similar to chromosomal tRNA
Arginine
SEQ ID NO: 165
ATCCCACCGNNNCCACCA chromosomal tRNA Leucine
SEQ ID NO: 166
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACC mitochondrial 16S rRNA
SEQ ID NO: 167
GCTAAACCTAGCCCCAAACCCACTCCACCTTACTACCAGACAACCTTAGCCGAACCAT
TTACCCAAATAAAGTATAGGCGATAGAAATTGAAACCT mitochondrial 16S rRNA
SEQ ID NO: 168
NTCCCGGNCNANNCNCCA chromosomal tRNA proline/glycine
SEQ ID NO: 169
NTCCCGGNCNNNNCNCCA chromosomal tRNA proline/glycine
SEQ ID NO: 170
TTCCCGGCCAACGCACCA chromosomal tRNA Glycine
SEQ ID NO: 171
GACTCTTAGCGGTGGATCACTCGGCTCGTGCGTCGATGAAGAACGCAGCTAGCTGCGA
GAATTAATGTGAATTGCAGGACACATTGATCATCGACACTTCGAACGCACTTGCGGCCCCGGGTTCCTCCC
GGGGCTACGCCTGTCTGAGCGTCGCT chromosomal 28S rRNA
SEQ ID NO: 172
TGCCCGCATCCTCCACCA chromosomal tRNA Alanine
SEQ ID NO: 173
GGGAATACCGGGTGCTGTAGGCTT chromosomal 5S rRNA
SEQ ID NO: 174
GTATAGTGGTGAGTATCCCCGCCTGTCNCGCGGGAGACCGGGGTTCGATTCCCCGACG
GGGAGCCA chromosomal tRNA Aspartate
SEQ ID NO: 175
GAGAAAGCTCNCAAGAANTNNTAACNTCNNNGNCNNNNNNNNNNNNN
mitochondrial tRNA Serine
SEQ ID NO: 176
CACCTCTTTACAGTGACCA mitochondrial tRNA Lysine
SEQ ID NO: 177
TTCCCGGCCAATGCACCA chromosomal tRNA Glycine
SEQ ID NO: 178
TTCCCGGTCAGGGAA(TGAGGTTTT) similar to chromosomal tRNA
Glutamate
SEQ ID NO: 179
GGACCACCAGGTTGCCTAAGGAGGGGTGAACCGGCCCAGGTCGGAAACGGAGCAGGTC
AAAACTCCCGTGCTGATCAGTAGTGGGATCGCGCCTGTGAATAGCCACTGCACTCCAGCCTGGGCAACATA
GCGAGACCCCGTCTCTA 7SL RNA
SEQ ID NO: 180
GTTCGGCATCAATATGGTGACCTCCCGGGAGCGGGGGACCACCAGGTTGCCTAAGGAG
GGGTGAACCGGCCCAGGTCGGAAACGGAGCAGGTCAAAACTCCCGTGCTGATCAGTAGTGGGATCGCGCCT
GTGAATAGCCACTGCACTCCAGCCTGGGCAACATAGCGAGACCCCGTCTCTT 7SL RNA
SEQ ID NO: 181
GGCGCGTGCCTGTAGTCCCAGCTACTCGGGAGGCTGAGGTGGGAGGATCGCTTGAGCC
CAGGAGTTCTGGGCTGTAGTGCGCTATGCCGATCGGGTGTCCGCACTAAGTTCGGCATCAATATGGTGACC
TCCCGGGAGCGGGGGACCACCAGGTTGCCTAAGGAGGGGTGAACCGGCCCAGGTCGGAAACGGAGCAGGTC
AAAACTCCCGTGCTGATCAGTAGTGGGATCGCGCCTGTGAATAGCCACTGCACTCCAGCCTGAGCAACATA
GCGAGACCCCGTCTCTTA 7SL RNA
SEQ ID NO: 182
GCTTAACACAAAGCACCCAACTTACACTTAGGAGATTTCAACTTAACTTGACCGCTCT
GACCA mitochondrial tRNA Valine
SEQ ID NO: 183
GACTCTTAGCGGTGGATCACTCGGCTCGTGCGTCGATGAAGAACGCAGCTAGCTGCGA
GAATTAATGTGAATTGCAGGACACATTGATCATCGACACTTCGAACGCACTTGCGGCCCCGGGTTCCTCCC
GGGGCTACGCCTGTCTGAGCGTCGCT chromosomal 28S rRNA
SEQ ID NO: 184
GCTTAACACAAAGCACCCAACTTACACTTAGGAGATTTCAACTTAACTTGACCGCTCT
GACCA mitochondrial tRNA Valine
SEQ ID NO: 185
TCAGGACACATTGATCATCGACACTTCGAACGCACTTGCGGCCCCGGGTTCCTCCCGG
GGCTACGCCTGTCTGAGCGTCGCT chromosomal 28S rRNA
SEQ ID NO: 186
ATCCCACCGCTGCCACCA chromosomal tRNA Leucine
SEQ ID NO: 187
GGGATCACTCGGCTCGTGCGTCNATGAANAACGCAGCTAGCTGCGAGAATTANTGTGA
ATTGCAGGACACATTGATCATCGACACTTCGAACGCACTTGCGGCCCCGGGTTCCTCCCGGGGCTACGCCT
GTCTGANCGTCGCT chromosomal 28S rRNA
SEQ ID NO: 188
GGGTTTCGTACGTAGCAGAGCAGCTCCCTCGCTGCGATCTATTGAAAGTCAGCCCTCG
ACACAAGGGTTTGT chromosomal 28S rRNA
SEQ ID NO: 189
TCCCCGGCATCTCCACCA chromosomal tRNA Alanine
SEQ ID NO: 190
GGCTGGTCCGAAGGTAGTGAGTTATCTCAATTGATTGTTCACAGTCAGTTACAGATCG
AACTCCTTGTTCTACTCTCTCCCCCCTTCTCACTACTGCACTTGACTAGTCTTA RNY1 RNA
SEQ ID NO: 191
GGCCGCCGTAGGCGAAGGTGAAGATGGCTGCCTCTGCCTTT chromosomal mRNA
MRPL35
SEQ ID NO: 192
TCCCCGGCATCTCCACCA chromosomal tRNA Alanine
SEQ ID NO: 193
ATCCCACCGNNNCCACCA chromosomal tRNA Leucine
SEQ ID NO: 194
NTCCCNGNNNNNGNNNCN chromosomal tRNA
SEQ ID NO: 195
GGATCACTCGGCTCGTGCGTCGATGAAGAACGCAGCTAGCTGCGAGAATTAATGTGAA
TTGCAGGACACATTGATCATCGACACTTCGAACGCACTTGCGGCCCCGGGTTCCTCCCGGGGCTACGCCTG
TCTGAGCGTCGCT chromosomal 28S rRNA
SEQ ID NO: 196
GGACCACCAGGTTGCCTAAGGAGGGGTGAACCGGCCCAGGTCGGAAACGGAGCAGGTC
AAAACTCCCGTGCTGATCAGTAGTGGGATCGCGCCTGTGAATAGCCACCGCACTCCAGCCTGAGCAACATA
GCGAGACCCCGTCTCTA 7SL RNA
SEQ ID NO: 197
GTATAGTGGTTAGTATCCCCGCCTGTCACGCGGGAGACCGGGGTTCAATTCCCCGACG
GGGAGCCA chromosomal tRNA Aspartate
SEQ ID NO: 198
GGATCACTCGGCTCGTGCGTCGATGAAGAACGCAGCTAGCTGCGAGAATTAATGTGAA
TTGCAGGACACATTGATCATCGACACTTCGAACGCACTTGCGGCCCCGGGTTCCTCCCGGGGCTACGCCTG
TCTGAGCGTCGCT chromosomal 28S rRNA
SEQ ID NO: 199
ATCCCGGACGAGCCCCCA chromosomal tRNA Proline
SEQ ID NO: 200
GGGCGGTGATGACCCCAACATGCCATCTGAGTGTCGGTGCTGAAATCCAGAGGCTGTT
TCTGAGCT snoRNA C/D box 95
SEQ ID NO: 201
ATCCCGGACNANCCCCCA chromosomal tRNA Proline
SEQ ID NO: 202
GAGAAAGCTCACAAGAACTGCTAACTCATGCCCCCATGTCTAACAACATGGCTTTCTC
ACCA mitochondrial tRNA Serine
SEQ ID NO: 203
GAGAAAGCTCACAAGAACTGCTAACTCATGCCCCCATGTCTAACAACATGGCTTTCTC
ACCA mitochondrial tRNA Serine
SEQ ID NO: 204
GACTCTTAGCGGTGGATCACTCGGCTCGTGCGTCGATGAAGAACGCAGCTAGCTGCGA
GAATTAATGTGAATTGCAGGACACATTGATCATCGACACTTCGAACGCACTTGCGGCCCCGGGTTCCTCCC
GGGGCTACGCCTGTCTGAGCGTCGCT chromosomal 28S rRNA
SEQ ID NO: 205
GTCACGCACCGCACGTTCGTGGGGAACCTGGCGCTAAACCATTCGTAGACGACCTGCT
TCTGGGTCGGGGTTTCGTACGTAGCAGAGCAGCTCCCTCGCTGCGATCTATTGAAAGTCAGCCCTCGACAC
AAGGGTTTGT chromosomal 28S rRNA
SEQ ID NO: 206
GGGTTTCGTACGTAGCAGAGCAGCTCCCTCGCTGCGATCTATTGAAAGTCAGCCCTCG
ACACAAGGGTTTGT chromosomal 28S rRNA
SEQ ID NO: 207
GTATAGTGGTGAGTATCCCCGCCTGTCACGCGGGAGACCGGGGTTCGATTCCCCGACG
GGGAGCCA chromosomal tRNA Aspartate
SEQ ID NO: 208
GCTTAACACAAAGCACCCAACTTACACTTAGGAGATTTCAACTTAACTTGACCGCTCT
GACCA mitochondrial tRNA Valine
SEQ ID NO: 209
GAGAAAGCTCACAAGAACTGCTAACTCATGCCCCCATGTCTAACAACATGGCTTTCTC
ACC mitochondrial tRNA Serine
SEQ ID NO: 210
TTCCCGGCCAATGCACCA chromosomal tRNA Glycine
SEQ ID NO: 211
TCCCCGGCATCTCCACCA chromosomal tRNA Alanine
SEQ ID NO: 212
ATCCCGGACGAGCCCCCA chromosomal tRNA Proline
SEQ ID NO: 213
ATCCCGGACGAGCCCCCA chromosomal tRNA Proline
SEQ ID NO: 214
TTCCCGGCCAATGCACCA chromosomal tRNA Glycine
SEQ ID NO: 215
TTCCCGGCCAATGCACCA chromosomal tRNA Glycine
SEQ ID NO: 216
GGATCACTCGGCTCGTGCGTCGATGAAGAACGCAGCTAGCTGCGAGAATTAATGTGAA
TTGCAGGACACATTGATCATCGACACTTCGAACGCACTTGCGGCCCCGGGTTCCTCCCGGGGCTACGCCTG
TCTGAGCGTCGCT chromosomal 28S rRNA
SEQ ID NO: 217
GGGTTTCGTACGTAGCAGAGCAGCTCCCTCGCTGCGATCTATTGAAAGTCAGCCCTCG
ACACAAGGGTTTGT chromosomal 28S rRNA
SEQ ID NO: 218
GTATAGTGGTGAGTATCCCCGCCTGTCACGCGGGAGACCGGGGTTCGATTCCCCGACG
GGGAGCCA chromosomal tRNA Aspartate
SEQ ID NO: 219
ATCCCGGACGAGCCCCCA chromosomal tRNA Proline
SEQ ID NO: 220
TGCCCGCATCCTCCACCA chromosomal tRNA Alanine
SEQ ID NO: 221
GCTTAACACAAAGCACCCAACTTACACTTAGGAGATTTCAACTTAACTTGACCGCTCT
GACCA mitochondrial tRNA Valine

### Example 4

### Subject 7

SEQ ID NO: 222
ATCGAATGGACTCGAATGGGATCATCGAATGGAATGCAATGGATTAGTCCATGGACTC
GAATTCAATCACCATCGAATACAATCGAATGGAGTCATCGAATCGACTCAAATGGAATAATCATTGAATGG
AATCGAATGGAATCATCGAGTGGAATCGAATGGAATCATGATCAAATGGAATCGAATGTAATCATCATCAA
ATGGAATCAAAAATAACCATCATCAATTGGTATTGAATGGAATTGTCATCAAATGGAATTCAAAGGAATCA
TCATCAAATGGAACCGAATGGAATCCTCATTGAATGGAAATGAAAGGAGTCATCATCTAATGGAATCGCAT
GGAA HSATII
SEQ ID NO: 223
GGAGCACCCAGATTCATAAAGCAAGTCCTTAGAGATCTACAAAGAGATTGAGACTCCC
ACAAAATAATAATGGGAGACTTTAACACCCCACTGTCAACATTGAACAGATCAATGAGACAGAAAGTTAAC
AAGGATATCCAGGAATTGAAGTCAGCTCTGCACCAAG L1
SEQ ID NO: 224
GCTGGGACTACAGGTGTGAGCCACCATGCCTGGCTAATTTTTGTATTTTTAGTAGAGA
C(A)GGGTTTCACCATGTTGGACAGGCTGTTCTTGAACTCCTGAAATCAGGTGATCCGCCCTCCTCAGCAT
C AluSx
SEQ ID NO: 225
AAACGCCTTCACAGCAACATTTAGACTACTGTTCAGCCAAGTATCTGGGCACCATAGC
TCAGATAAGTTGACGTATAAAATTCACCATCACAAGGGAGGAATTTATATATACACTTTATTGCAGCAAAA
TCTTTACAGTTTAACGTTTTGTACTTTTCCCAGAAGGAAACGTTTCAGTGCAGAGTTGAATA MLT2C2
SEQ ID NO: 226
TCTACGGCCATACCACCCTGAACGCGCCCGATCTCGTCTGATCTCGGAAGCTAAGCAG
GGTCGGGCCTGGTTAGTACTTGGATGGGAGACCGCCTGGGAATACCGGGTGCTGTAGGCTT
chromosomal 5S rRNA
SEQ ID NO: 227
TACCCACAGAACCCATAGACACATGCACAGACATGGATACCCGGGAACACACAGTTAC
ACACATTCACACACAAACACTCATGCATACAACATTACAGGCTCTCACAGCCTGAAAGACACACAGGACCA
CCCCCAGAAACACACACAGACATACACAGCCACAGCCACCTG ERV1-N3-I_DR
SEQ ID NO: 228
CCACCCACTCCCTTCTCTCCACATCCCCTATGACCACCCACTGGCACCTGCTCTTTAT
TATGGATTCTGTAACTTCTTTAATTACATCACCGTCTACCTAGTTGCTCACTTAGAAACCTCATTCATCAG
TGATTCTTCCATTACCCTCACCTAATCCCATCAAGGCCATCTCCTTCTTCAATGACCAGGTGTCCCATGTT
CCCCTTGGTCTTAATCCTGTATACAGCTTATGCTAGTCCCTTAGCCTATATCATGGTTTCATTTGAAAAAG
GGCCATAACATCTCCTGATTTACTTGTACTTTTCTATGATTACCACTGATGTTGAACATTTTTTATATATC
AGCTATTTCACAATGATA L1ME4A
SEQ ID NO: 229
GCAGGTCCACTAGGTCTAAGGCCAAAGAGGTGAGGAAAGCTGTCACAGGGAAAAACAT
GGAATGAACAGGAGTAAGTTGTTCCCATAAATCTTCTGTCCTTGAATTAATTTTATTCCCTATCCTATCCC
ATGCCATTCTCTAATGCCAACTGAGTCCGGGTG MamRep1527
SEQ ID NO: 230
AAATCATCATTCTCAACAAACTAACACAAGAACAGAAAACCAAATACCACATGTTCTC
ACTCATAAATGGGAGTTGAACAATGAGAACAATGGACACAGGGAGGGGAACATCACACACCAGGGCCTGTC
AGAGGGTGAGGGACTAGGGGAGGGATAGCATT L1PA7
SEQ ID NO: 231
GGTTCCCCCCTCCTCTCCTCTCCCTGGCCTTGTCCCCAGGATGCGGATCAGCACTTCC
CTCTCCATCACCACTTCACACCCCTGTCTCCAGCCGGCCCCATGTGTCTCCCACCCCATCTCTCCATGTCC
CTCAACAGTGTCTCTGCATCGCTGTCTGCCCTAG Gypsy-25_LBS-I
SEQ ID NO: 232
AGATAATAACACTTAGTGGGTAAGAGGAGTTATTTATAAAAAATAATAATAATAAAGG
AAGCAACAAAGCCTAGTGCCCATTAGTTATTTTTCCTGCTCTTCTCCCTCCTGCCACCCTCCACCCTTTG
L1PA13
SEQ ID NO: 233
ACACACATGCACGGCCTCCCCCATTATCAACACCCTGCACCCTGAATAGTGCATTTAC
AACAGTGATGACCCTACACGGCCACACCACGAGCACCCGAACCCCACACGGCCACACCACGAGCACCCGAA
CCCTACACGGCCACACCACGAGCACCCGAACCCTACACGGCCACACCACGAG L1MC1_EC
SEQ ID NO: 234
TGCTCTTCCTTCCAGACCTAACTCCAGGAAGTGTCCCTCTCCAGGAAGCTCCTCTGAT
CCCCACTGGAAAGGTGTGCCTCCCCTTTGTGCCCTTTGA Unknown Function Chr1
SEQ ID NO: 235
GCTGGGANTACAGGTGTGAGCCACCATGCCTGGCTAATTTTTGTATTTTTAGTANAGA
CGGGGTTTCACCATGTTGGACAGGCTGTTCTTGAACTCCTGAAATCAGGTGATCCGCCCTCCTCAGCATC
AluSx
SEQ ID NO: 236
GGNTGTGTGGCATGCTAACAAACATGGGTGAGATAACGAGGGTTCAGGAAATCTTACT
TTCCATTTCTGTCATTGTTGTTGCCTATGGTCTGATTCCTTTCTCTTAATGATGCAGTAAAGAGGTAGCAA
ATACAGCAAAGTGCTTCATAAGTCAGGCTCAG Rover_DM

### Subject 1

SEQ ID NO: 237
TTTCTATCTACTTCAAATTCCTCCCTGTACGAAAGGACAAGAGAAATAAGGCCTACTT
CACAAAGCGCCTTCCCCCGTAAATGATATCATCTCAACTTAGTA
mitochondrial 16S rRNA
SEQ ID NO: 238
GATATAGACAGCAGGACGGTGGCCATGGAAGTCGGAATCCGCTAAGGAGTGTGTAACA
ACTCACCTGCCGAATCAACTAGCCCTGAAAATGGATGGCGCTGGAGCGTCGGGCCCATACCCGGCCGTCGC
CGGCAGTCGAGAG chromosomal 28S rRNA
SEQ ID NO: 239
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTAC mitochondrial 16S rRNA
SEQ ID NO: 240
CTGCTTTGGAGTTCTGTTCCAGTTCCTTAG(C)CCCAGAACACTTTTAGGTTCTCCAT
CTCCTA Unknown Function Chr13
SEQ ID NO: 241
GCTGTAGTGGCTTCGTCTTCGGTTTTTCTCTTCCTTCGCTAACGCCTCCCGGCTCTCG
TCAGCCTCCCGCCGGCCGTCTCCTTAACACCG(TA)
chromosomal mRNA SKP1
SEQ ID NO: 242
GAGCCGCCTGGATACCGCAGCTAGGAATAATGGAATAGGACCGCGGTTCTATTTTGTT
GGTTTTCGGAACTGAGGCCATGATTAAGAGGGACGGCCGGGGGCATTCGTATTGCGCCGCTAGAGGTGAAA
TTCTTGGACCGGCGCAAGACGGACCAGAGCGAAAGCATTTGCCAAGAATGTTTTCATTAATCAAGAACGAA
AGTCGGAGGTTCGAAGACGATCAGATACCGTCGTAGTTCCGACCATAAACGATGCCGACCGGCGATGCGGC
GGCGTTA chromosomal 18S rRNA
SEQ ID NO: 243
GGCGAAAGACTAATCGAACCATCTAGTAGCTGGTTCCCTCCGAAGTTTCCCTCAGGAT
AGCTGGCGCTCTCGCAGACC chromosomal 28S rRNA
SEQ ID NO: 244
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 245
AACCTGGCGCTAAACCATTCGTAGACGACCTGCTTCTGGGTCGGGGTTTCGTACGTAG
CAGAGCAGCTCCCTCGCTGCGATCTATTGAAAGTCAGCCCTCGA
chromosomal 28S rRNA
SEQ ID NO: 246
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 247
GCGCTAAACCATTCGTAGACGACCTGCTTCTGGGTCGGGGTTTCGTACGTAGCAGAGC
AGCTCCCTCGCTGCGATCTATTGAAAGTCAGCCCTCGACACAAGGGTTTGT
chromosomal 28S rRNA
SEQ ID NO: 248
AGGAGGAGGCGCAGCTTACAGAGACGGTGCCCCTTGCAGGCACAACCACTAGCAAGTC
CCGGGGGCACCGTTCCTTGAAATAGGAAGACCCGCTCGCTCCAGGCAGAGCTGCCTGAAGGGCAAGCACCC
AGAGTGGGGAAGGAAAGAAGAGCCCCGAAGAGGCAGGAGAAAGGCCCACTTTGAGAGCTCACA(GCAGTTG
AACATGGGTCAGTCGGTCCTGAGAGATGG) Unknown Function Chr10
SEQ ID NO: 249
TCGCCCGTCACGCACCGCACGTTCGTGGGGAACCTGGCGCTAAACCATTCGTAGACGA
CCTGCTTCTGGGTCGGGGTTTCGTACGTAGCAGAGCAGCTCCCTCGCTGCGATCTATTGAAAGTCAGCCCT
CGACACAAGGGTTTGT chromosomal 28S rRNA
SEQ ID NO: 250
GAACTGGCGCTGCGGGATGAACCGAACGCCGGGTTAAGGCGCCCGATGCCGACGCTCA
TCAGACCCCAGAAAAGGTGTTGGTTGATATAGACAGCAGGACGGTGGCCATGGAAGTCGGAATCCGCTAAG
GAGTGTGTAACAACTCACCTGCCGAATCAACTAGCCCTGAAAATGGATGGCGCTGGAGCGTCGGGCCCATA
CCCGGCCGTCGCCGGCAGTCGAGA chromosomal 28S rRNA
SEQ ID NO: 251
GACGGTGGCCATGGAAGTCGGAATCCGCTAAGGAGTGTGTAACAACTCACCTGCCGAA
TCAACTAGCCCTGAAAATGGATGGCGCTGGAGCGTCGGGCCCATACCCGGCCGTCGCCGGCAGTCGAG
chromosomal 28S rRNA
SEQ ID NO: 252
TCAGACCGGAGTAATCCAGGTCGGTTTCTATCTACTTCAAATTCCTCCCTGTACGAAA
GGACAAGAGAAATAAGGCCTACTTCACAAAGCGCCTTCCCCCGTAAATGATATCATCTCAACTTAGTATTA
TACCCACACCCACCCAAGAACAGGGTTTGAAAA mitochondrial 16S rRNA

### Subject 2

SEQ ID NO: 253
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGA
chromosomal 28S rRNA
SEQ ID NO: 254
GACCACCAGGTTGCCTAAGGAGGGGTGAACCGGCCCAGGTCGGAAACGGAGCAGGTCA
AAACTCCCGTGCTGATCAGTAGTGGGATCGCGCCTGTGAATAGCCACTGCACTCCAGCCTGAGCAACATAG
CGAGACCCCGTCTCT(TA) 7SL RNA
SEQ ID NO: 255
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACTATA
CTCAATTGATCCAATAACTTGACCAACGGAACAAGTTACCCTAGGGATAACAGCGCAATCCTATTCTAGAG
TCCATATCAACAATAGGGTTTACGACCTCGATGTTGGATCAGGACATCCCAATGGTGCAGCCGCTATTAAA
GGTTCGTTTGTTCAACGATTAAAGTCCTACGTGATCTGAGTTCAGACCGGAGTAATCCAGGTCGGTTTCTA
TCTACTTCAAATTCCTCCCTGTACGAAAGGACAAGAGAAATAAGGCCTACTTCACAAAGCGCCTTCCCCCG
TAAATGATATCATCTCAACTTAGTATTATACCCACACCCACCCAAGAACAGGGTTT(AAAA)
mitochondrial 16S rRNA
SEQ ID NO: 256
ACGCACCGCACGTTCGTGGGGAACCTGGCGCTAAACCATTCGTAGACGACCTGCTTCT
GGGTCGGGGTTTCGTACGTAGCAGAGCAGCTCCCTCGCTGCGATCTATTGAAAGTCAGCCCTCGACACAAG
GGTT chromosomal 28S rRNA
SEQ ID NO: 257
GAACTGGCGCTGCGGGATGAACCGAACGCCGGGTTAAGGCGCCCGATGCCGACGCTCA
TCAGACCCCAGAAAAGGTGTTGGTTGATATAGACAGCAGGACGGTGGCCATGGAAGTCGGAATCCGCTAAG
GAGTGTGTAACAACTCACCTGCCGAATCAACTAGCCCTGAAAATGGATGGCGCTGGAGCGTCGGGCCCATA
CCCGGCCGTCGCCGGCAGTCGAGA chromosomal 28S rRNA
SEQ ID NO: 258
GTGCGGAGTGCCCTTCGTCCTGGGAAACGGGGCGCGGCTGGAAAGGCGGCCGCCCCCT
CGCCCGTCACGCACCGCACGTTCGTGGGGAACCTGGCGCTAAACCATTCGTAGACGACCTGCTTCTGGGTC
GGGGTTTCGTACGTAGCAGAGCAGCTCCCTCGCTGCGATCTATTGAAAGTCAGCCCTCGACACAAGGGTTT
GT chromosomal 28S rRNA
SEQ ID NO: 259
GAAGTCGGAATCCGCTAAGGAGTGTGTAACAACTCACCTGCCGAATCAACTAGCCCTG
AAAATGGATGGCGCTGGAGCGTCGGGCCCATACCCGGCCGTCGCCGGCAGTCGAG
chromosomal 28S rRNA
SEQ ID NO: 260
TGCTTGGCTGAGGAGCCAATGGGGCGAAGCTACCATCTGTGGGATTATGACTGAACGC
CTCTAAGTCAGAATCCCGCCCAGGCGGAACGATACGGCAGCGCCGCGGAGCCTCGGTTGGCCTCGGATAGC
CGGTCCCCCGCCTGTCCCCGCCGGCGGGCCGCCC(A)
chromosomal 28S rRNA
SEQ ID NO: 261
GTTAAATACAGACCAAGAGCCTTCAAAGCCCTCAGTAAGTTGCAATACTTAATTTCTG
CCA mitochondrial tRNA Tryptophan
SEQ ID NO: 262
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGAGA
chromosomal 28S rRNA
SEQ ID NO: 263
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACTATA
CTCAATTGATCCAATA mitochondrial 16S rRNA
SEQ ID NO: 264
TTCAACGATTAAAGTCCTACGTGATCTGAGTTCAGACCGGAGTAATCCAGGTCGGTTT
CTATCTACTTCAAATTCCTCCCTGTACGAAAGGACAAGAGAAATAAGGCCTACTTCACAAAGCGCCTTCCC
CCGTAAATGATATCATCTCAACTTAGTATTATACCCACACCCACCCAAGAACAGGGTT(ACCA)
mitochondrial 16S rRNA
SEQ ID NO: 265
CTTAGCTGTTAACTAAGTGTTTGTGGGTTTAAGTCCCATTGGTCTAGCCA
mitochondrial tRNA Asparagine
SEQ ID NO: 266
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 267
ACGCTCATCAGACCCCAGAAAAGGTGTTGGTTGATATAGACAGCAGGACGGTGGCCAT
GGAAGTCGGAATCCGCTAAGGAGTGTGTAACAACTCACCTGCCGAATCAACTAGCCCTGAAAATGGATGGC
GCTGGAGCGTCGGGCCCATACCCGGCCGTCGCCGGCAGTCGAG
chromosomal 28S rRNA
SEQ ID NO: 268
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTA
mitochondrial 16S rRNA

### Subject 4

SEQ ID NO: 269
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 270
TAGGGACCTGTATGAATGGCTTCACGAGGGTTCANCTGTCTCTTACTTTTAACCAGTG
AAATTGACCTGCCCGTGAAGAGGCGGGCATGACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACT
mitochondrial 16S rRNA
SEQ ID NO: 271
TCGCCCGTCACGCACCGCACGTTCGTGGGGAACCTGGCGCTAAACCATTCGTAGACGA
CCTGCTTCTGGGTCGGGGTTTCGTACGTAGCAGAGCAGCTCCCTCGCTGCGATCTATTGAAAGTCAGCCCT
CGACACAAGGGTTTGT chromosomal 28S rRNA
SEQ ID NO: 272
GGTCGGGGTTTCGTACGTAGCAGAGCAGCTCCCTCGCTGCGATCTATTGAAAGTCAGC
CCTCGACACAAGGGTTTGT chromosomal 28S rRNA
SEQ ID NO: 273
GAACTGGCGCTGCGGGATGAACCGAACGCCGGGTTAAGGCGCCCGATGCCGACGCTCA
TCAGACCCCAGAAAAGGTGTTGGTTGATATAGACAGCAGGACGGTGGCCATGGAAGTCGGAATCCGCTAAG
GAGTGTGTAACAACTCACCTGCCGAATCAACTAGCCCTGAAAATGGATGGCGCTGGAGCGTCGGGCCCATA
CCCGGCCGTCGCCGGCAGTCGAGAG chromosomal 28S rRNA
SEQ ID NO: 274
GCTTAACACAAAGCACCCAACTTACACTTANGAGATTTCAACTTAACTTGACCGCTCT
GACCA mitochondrial tRNA Valine
SEQ ID NO: 275
AGACCGGAGTAATCCAGGTCGGTTTCTATCTACTTCAAATTCCTCCCTGTACGAAAGG
ACAAGAGAAATAAGGCCTACTTCACAAAGCGCCTTCCCCCGTAAATGATATCATCTCAACTTAG(A)
mitochondrial 16S rRNA
SEQ ID NO: 276
GAACTGGCGCTGCGGGATGAACCGAACGCCGGGTTAAGGCGCCCGATGCCGACGCTCA
TCAGACCCCAGAAAAGGTGTTGGTTGATATAGACAGCAGGACGGTGGCCATGGAAGTCGGAATCCGCTAAG
GAGTGTGTAACAACTCACCTGCCGAATCAACTAGCCCTGAAAATGGATGGCGCTGGAGCGTCGGGCCCATA
CCCGGCCGTCGCCGGCAGTCGAGAGTG(TT) chromosomal 28S rRNA
SEQ ID NO: 277
TAACTTGACCAACGGAACAAGTTACCCTAGGGATAACAGCGCAATCCTATTCTAGAGT
CCATATCAACAATAGGGTTTACGACCTCGATGTTGGATCAGGACATCCCGATGGTGCAGCCGCTATTAAAG
GTTCGTTTGTTCAACGATTAAAGTCCTACGTGATCTGAGTTCAGACCGGAGTAATCCAGGTCGGTTTCTAT
CTACTTCAAATTCCTCCCTGTACGAAAGGACAAGAGAAATAAGGCCTACTTCACAAAGCGCCTTCCCCCGT
AAATGATATCATCTCAACTTAGTATTATACCCACACCCACCCAAGAACAGGGTTTCAA
mitochondrial 16S rRNA
SEQ ID NO: 278
GAACTGGCGCTGCGGGATGAACCGAACGCCGGGTTAAGGCGCCCGATGCCGACGCTCA
TCAGACCCCAGAAAAGGTGTTGGTTGATATAGACAGCAGGACGGTGGCCATGGAAGTCGGAATCCGCTAAG
GAGTGTGTAACAACTCACCTGCCGAATCAACTAGCCCTGAAAATGGATGGCGCTGGAGCGTCGGGCCCATA
CCCGGCCGTCGCCGGCAGTCGAGA chromosomal 28S rRNA
SEQ ID NO: 279
GGTCAAAACTCCCGTGCTGATCAGTAGTGGGATCGCGCCTGTGAATAGCCACTGCACT
CCAGCCTGAGCAACATAGCGAGACCCCGTCTCTGA 7SL RNA
SEQ ID NO: 280
GGACCGGGGTCCGGTGCGGAGTGCCCTTCGTCCTGGGAAACGGGGCGCGGCTGGAAAG
GCGGCCGCCCCCTCGCCCGTCACGCACCGCACGTTCGTGGGGAACCTGGCGCTAAACCATTCGTAGACGAC
CTGCTTCTGGGTCGGGGTTTCGTACGTAGCAGAGCAGCTCCCTCGCTGCGATCTATTGAAAGTCAGCCCTC
GACACAAGGGTTTGT chromosomal 28S rRNA
SEQ ID NO: 281
TTGGTTGATATAGACAGCAGGACGGTGGCCATGGAAGTCGGAATCCGCTAAGGAGTGT
GTAACAACTCACCTGCCGAATCAACTAGCCCTGAAAATGGATGGCGCTGGAGCGTCGGGCCCATACCCGGC
CGTCGCCGGCAGTCGAGAGT chromosomal 28S rRNA
SEQ ID NO: 282
TCGCCCGTCACGCACCGCACGTTCGTGGGGAACCTGGCGCTAAACCATTCGTAGACGA
CCTGCTTCTGGGTCGGGGTTTCGTACGTAGCAGAGCAGCTCCCTCGCTGCGATCTATTGAAAGTCAGCCCT
CGACACAAGGGTTTGT chromosomal 28S rRNA
SEQ ID NO: 283
ATGCCGACGCTCATCAGACCCCAGAAAAGGTGTTGGTTGATATAGACAGCAGGACGGT
GGCCATGGAAGTCGGAATCCGCTAAGGAGTGTGTAACAACTCACCTGCCGAATCAACTAGCCCTGAAAATG
GATGGCGCTGGAGCGTCGGGCCCATACCCGGCCGTCGCCGGCAGTCGAG
chromosomal 28S rRNA
SEQ ID NO: 284
GGGAACCTGGCGCTAAACCATTCGTAGACGACCTGCTTCTGGGTCGGGGTTTCGTACG
TAGCAGAGCAGCTCCCTCGCTGCGATCTATTGAAAGTCAGCCCTCGACACAAGGGTTTGT
chromosomal 28S rRNA

### Subject 5

SEQ ID NO: 285
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACTATA
CTCAATTGATCCAATAACTTGACCAACGGAACAAGTTACCCTAGGGATAACAGCGCAATCCTATTCTAGAG
TCCATATCAACAATAGGGTTTACGACCTCGATGTTGGATCAGGACATCCCAATGGTGCAGCCGCTATTAAA
GGTTCGTTTGTTCAACGATTAAAGTCCTACGTGATCTGAGTTCAGACCGGAGTAATCCAGGTCGGTTTCTA
TCTACTTCAAATTCCTCCCTGTACGAAAGGACAAGAGAAATAAGGCCTACTTCACAAAGCGCCTTCCCCCG
TAAATGATATCATCTCAACTTAGTATTATACCCACACCCACCCAAGAACAGGGTTTAAAA
mitochondrial 16S rRNA
SEQ ID NO: 286
TGCTTGGCTGAGGAGCCAATGGGGCGAAGCTACCATCTGTGGGATTATGACTGAACGC
CTCTAAGTCAGAATCCCGCCCAGGCGGAACGATACGGCAGCGCCG
chromosomal 28S rRNA
SEQ ID NO: 287
ACAGCAGGACGGTGGCCATGGAAGTCGGAATCCGCTAAGGAGTGTGTAACAACTCACC
TGCCGAATCAACTAGCCCTGAAAATGGATGGCGCTGGAGCGTCGGGCCCATACCCGGCCGTCGCCGGCAGT
CGAG chromosomal 28S rRNA
SEQ ID NO: 288
TGCTTGGCTGAGGAGCCAATGGGGCGAAGCTACCATCTGTGGGATTATGACTGAACGC
CTCTAAGTCAGAATCCCGCCCAGGCGGAACGATACGGCAGCGCCGCGGAGCCTCGGTTGGCCTCGGATAGC
CGGTCCCCCG chromosomal 28S rRNA
SEQ ID NO: 289
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACA mitochondrial 16S rRNA
SEQ ID NO: 290
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGAGAG
chromosomal 28S rRNA
SEQ ID NO: 291
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGAGA
chromosomal 28S rRNA
SEQ ID NO: 292
AACCTGGCGCTAAACCATTCGTAGACGACCTGCTTCTGGGTCGGGGTTTCGTACGTAG
CAGAGCAGCTCCCTCGCTGCGATCTATTGAAAGTCAGCCCTCGACACAAGGGTTTGT
chromosomal 28S rRNA
SEQ ID NO: 293
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGAG
chromosomal 28S rRNA
SEQ ID NO: 294
ACGCTCATCAGACCCCAGAAAAGGTGTTGGTTGATATAGACAGCAGGACGGTGGCCAT
GGAAGTCGGAATCCGCTAAGGAGTGTGTAACAACTCACCTGCCGAATCAACTAGCCCTGAAAATGGATGGC
GCTGGAGCGTCGGGCCCATACCCGGCCGTCGCCGGCAGTCGAGA
chromosomal 28S rRNA
SEQ ID NO: 295
TGTCTGTAGAAAAAGATTGGGATGATTTGTGGTTAGGAGTGA
Unknown Function Chr21
SEQ ID NO: 296
GACGCTCATCAGACCCCAGAAAAGGTGTTGGTTGATATAGACAGCAGGACGGTGGCCA
TGGAAGTCGGAATCCGCTAAGGAGTGTGTAACAACTCACCTGCCGAATCAACTAGCCCTGAAAATGGATGG
CGCTGGAGCGTCGGGCCCATACCCGGCCGTCGCCGGCAGTCGAG
chromosomal 28S rRNA
SEQ ID NO: 297
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 298
ACGCTCATCAGACCCCAGAAAAGGTGTTGGTTGATATAGACAGCAGGACGGTGGCCAT
GGAAGTCGGAATCCGCTAAGGAGTGTGTAACAACTCACCTGCCGAATCAACTAGCCCTGAAAATGGATGGC
GCTGGAGCGTCGGGCCCATACCCGGCCGTCGCCGGCAGTCGAG
chromosomal 28S rRNA
SEQ ID NO: 299
TAACACAAAGCACCCAACTTACACTTAGGAGATTTCAACTTAACTTGACCGCTCTGAC
CA mitochondrial tRNA Valine
SEQ ID NO: 300
GAGCAGGTCAAAACTCCCGTGCTGATCAGTAGTGGGATCGCGCCTGTGAATAGCCACT
GCACTCCAGCCTGGGCAACATAGCGAGACCCCGTCTCTT 7SL RNA

### Subject 6

SEQ ID NO: 301
NNACCCACCATCTCATCCTTCTCTGCCCGGCGTTTGCCCATCTTCCACTGCTTGTCAT
CCAGGCAGCTGAGGAAATGCTGGAAGCCTTCGTACTGGGAGAGCACAGGGTGGCTGGTCATGTGGTCCATC
CAGAGGATGAGTCTCCGCTTCCGCTTTTCGATGAAGTCCTAAATTTGGGTAACAATAGCTTCAGTGAAAGT
TAGGAGAATTGTATTTTAGTGG Gypsy-614 AA-I
SEQ ID NO: 302
NANTCATGCAGGTGTTATTAACTTTANNNNNNTCNATCTCAACTAGATCACTGTCAGG
GACGATGTGGTAACAATTGATTTTTCCAGGTCTGAGAGAATTCTCAGTTTACTTATTTTCATTTCTCCATA
GACTAATTTTTTTCCTTTGCTTGGAGATTTATGGCAGCATTTCTTTCTTCTTGCTCTCTCATTTCAATTTG
AAAATAGCATGCTTTTAAGCACAATATTTGGGGAGGAAAAGCAAAGTGACTTANAGGCTTCAAAAACAACT
CATCCAAGCCCATAAATTTGGCAGCCTAAACAGGCATTGACAAAGAAGGATGTACTCTTTCTGAACCTTAT
GCATTTCAACCATGTG Unknown Function Chr11
SEQ ID NO: 303
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 304
NCAGAGAATCAACATTTTGCCCAGAGNNNNNNNNNNANNGAAGTAGGGGGTGGGGGGT
TCTCCAAAGTCNNNNNNNNTTCCACGAGAGCATGCTGCCCAAACAGGTTCAGAATTCTGTAGATACAGAAT
AGGAAGAAAGGATGTCAGAGGAAGGAACAACATAGGCATGAAGGTGCATTGCATTAGGGAAACATGGCGTG
TCA Unknown Function Chr8
SEQ ID NO: 305
TAATGGCCAAAGTCAGTTTCTGCCTGTGGCTATGGTGTAGCGTTTTCTTTTTAGTCCT
TGGTGAGTACTGGACATTTGTAATATGTAAGCATGGTACTTCACAGAAGACTTAGCAGTTTACATGCATCT
GGTAGTTTATTTAGCATTTGTGAAAAATAAGAATTTTGTTATTTCAGAGTTTTTAAAAAGTTACTCAGCGA
ATTTTTGTTTGTTTTTAGGGAACAGCAGAAACTCATGAGCTGGCAGAAGGCAGTACTGCTGATGTTCTGCA
TTCGAGAATCAGTGGTGAAATAATGGAATTAGTCCTGGTGAAATACCAGGGCAAAAACTGGAATGGACATT
TCCGCATACGTGATACACTACCAGAATTCTTTCCTGTGTGTTTTTCTTCTGACTCCACAGAAGTGACGACA
GTCGACCTGTCAGTCCACGTCAGGAGAATTGGCAGCCGGATGGTGCTGTCTGTCTTTAGTCCCTATTGGTT
AATCAACAAGACTACCCGGGTTCTCCAGTATCGTTCAGAAGATATTCATGTGAAACATCCAGCTGATTTCA
Unknown Function Chr15
SEQ ID NO: 306
TGCTGGGTTCCCTTACCCCAAGACATGGATTCTTAATGACCCTGGAGCCCTGTGATTT
AGGATGCACTCAAGAAATGCAGGCTGAACATGATTGGTTTGTTACCCATCTGGAAAGAGGGGATAAAGGCA
TCTCTTTATTCATCTCTCTTTCCAGTGAATACCTGGCGTATGTGACAGAGAGAATAAAAAATGTCCTTTCT
TCTTCCAAACTTATATCCCTGAGTCCTGGCAACCTGTGCAGGTGTTAACTGTGGGTGCAAGTGTAACATTC
AGCCATGAAGCAGGAGGCATAGCTGACAGGAATATTTGCACTCATCTGAGCGGAGCCTAAGCCCACCTGCT
GTCAGTAACCATTGAATTCTCTAGACC MER52AI
SEQ ID NO: 307
CTGGGCACACAGCAAGTGTCCTGGACATGCAGCTCGGGTCTGCAGATAGAGCGGCAGC
ATGGGATTGTGGGAGGAGCTTGGGTGTGAGGGCCGTGTAGACAGCTTAGTTTTGTGACCTTGCGCAGGTAA
TTTCTGTTCTCTGAGCCTCAATTCCATTGCCTTTAAAATGAGA MIRc
SEQ ID NO: 308
GCCCCTAGTAACTTCTAATTCTCTGTCTCTAGGACTCTGCTTATTCTAGATCTTTCAT
GTAAGTGGGATCATACAATACGTGTCTTTCTGTGTTTAACTTATTTCACTTAGACGAATGTTTGCAAGGTT
TGCCTGTGCTATAGCATGTGTCAATACTTCATTCCCTTTTATGGATGAATAATATCCTATTGTATGCATAT
ACTGTACCATATTTTGTTTATCCATTCATCGGTTGATGAACATATTGGTTATTTCCACCGCCTTTTAATTA
TTGTGAATAATGTTGTAATGAATAAGGCTGTACAAATATCTGTTCATTCGCTGTTGTTTTCAATTCTTTAG
GGTGTACCTACCAATGAAATTGCTG L1MB4
SEQ ID NO: 309
TACTAGAGTTTATGCTAATAAGGTGACTCTTGGAAGATGGGGGCTGGCTGCTAGAGGA
ACCATGATCCAAATTTTCACTGCCCCCACCACCATCTCCGGGAAAAGGAAAGGGACTGGAGACGGGAGAGG
AGCTAGGAATTGAGTTAATCACCAATGCCCAATGATTAATCATTAATTAATCATTAATCATTCCTAGGTAA
TGTGACCTCCATTAAAAAAAAAATCTTAAAGGGCAGGGTTCAGAGAACTTCTGGGATGGCAAACATATGCC
TCTACCAGGAGGATGGTATACCCCAACTTCACAGAGACAGAAGCTCCTGCACCCAGGACCCTTCTAGACCT
TGCCCTACATACCTCTTAGTCTGACTATTCAGTTGTATCCCTTATAATGTTCTTTTTAAAAACCAATAAAT
GTAAGTAAAGTATTTCCCTGAGTTCTGTGAGCCTTTACAGCAAATTACTGAACATGAGCAGGTGATCATGA
GACCCCTCAAGTTATAACCAAAAGTACCCTGGCAACTTAGGACT MER21C
SEQ ID NO: 310
(GCCCCGANAAATTCCA)CAGCGATATGGGGGCCTGGACCTTGCCTTCCCATCCTCCT
GGTGTGTGGCTTTCCCTAAGGGGCAACCTGTGGTTTCTGGTGGGTTGGTGGGTGAAATAAAGAGCCTGCAG
GGAGTANCTGGGGGATGGGAAGTGTGAGAAGACTGATGATTTCNNAGAGA
Unknown Function Chr1
SEQ ID NO: 311
CAAGAGGGTCGTTTGACCCTGGTGGGTCCTTTCCCTACCCGGTGCCTTTCTCGCCCGT
AGAAGGAGACCAGGTTCGGTTAAGCAGAGCAGAAACTATTCACTGATCAAGGAATGGAGTAGGAGAGTTCC
TGCTCAAAGTGCCTGGGGTGTAGTGTGGGGGTGCTCCTTAAGGTCT LTR75B_EC
SEQ ID NO: 312
TGCTTGGCTGAGGAGCCAATGGGGCGAAGCTACCATCTGTGGGATTATGACTGAACGC
CTCTAAGTCAGAATCCCGCCCAGGCGGAACGATACG
chromosomal 28S rRNA
SEQ ID NO: 313
CCACATAAGGGAGAGAGCACAGAAGAGGCNGGNNTTTTCCTAAGAAGGGATCACCCCA
CCTTGTAACCACATCTAGAACCCAGAAGCCCAAATGTCAAGATAACATTCCCTCGGTCAGGAGTACACGAA
GGACAAACCTCCAAAGACAGACGCAGCTGTCTAAAACCAAAGACGCTTGATGAATGCCGGCCACTGCGCGC
TTGGTAGCTAACACAGACAGTGGTGCGGCAGGTGGCATGGTGCCCCACAACATCCTAACCCTCAAAACCTG
TGAATGTCA Unknown Function Chr21

### Example 5

### Subject 3

SEQ ID NO: 314
TTCCCCCCTTCTCACTACTGCACTTGACTATA
Unknown Function Chr5
SEQ ID NO: 315
AACACGGACCAAGGAGTCTAACACGTGCGCGAGTCGGGGGCTCGCACGAAAGCCGCCG
TGGCGCAATGAAGGTGAAGGCCGGCGCGCTCGCCGGCCGAGGG
chromosomal 28S rRNA
SEQ ID NO: 316
CAGACCCCAGAAAAGGTGTTGGTTGATATAGACAGCAGGACGGTGGCCATGGAAGTCG
GAATCCGCTAAGGAGTGTGTAACAACTCACCTGCCGAATCAACTAGCCCTGAAAATGGATGGCGC
chromosomal 28S rRNA
SEQ ID NO: 317
AATTCNNNTNNNNACNCCAGTGTTTAAACTTTTANTNNNNACCATGNNNGGTCAATTA
TAACACATTAAAAAGTGACAACAGCTATAAGCTGCANTTTTACATTTGTAAANNNNAAACGTCCATTTTCA
AAAGCTGAACATAACTATCAAATTGTAATANCTAAGCTTNNNNNNNACTGCTTATACAGATGAAATTTTCT
ATGANNNANNATGATTTTATATCTGNCCACATTCTGNGATAGTTATTCTTCGACNAACANACCCTTTGGGA
GAACCAAATGATTCANNCNTTTTCACAGTATCCATACCATCCTTAANNNACCCAAATACTANNTGNTTAAA
GTCCANATGTTCTGCTTTCTTCANTGTTATAACAAATTGANAATTATTGNNNNNNTGGCNNTGNTTGGCCA
TGGATANNANANCANNACCAGTATGTTTCACATNANAATTTTNATCTTCNAATTTGNNNNNATNANNGGAC
TGTCNNNCTGTTCCATCATGTNTGGTGATATCTCCTCCTTGGNAAACAAAATCTGNAATTACTCTGNNANA
AA chromosomal mRNA RANBP2
SEQ ID NO: 318
AATTCCAAAANTGGAACTGCACTTGAAATTCGAATAGAANGAACTGTGTACTGTGATG
AAACTGCTGACGAATCCTCANGAATTAATGTGCATCAACCCACTGCTTTTGCTCACAAGTTACTTCAGCTC
TCTGGAGTGTCTCTCTTCTGGGATGAGTTTTCTGCATCANCCAAATCTTCCCCAGTGTGTTCAACTGCACC
AGTGGAAACTGAGCCAAAGCTCTCACCTANCTGGAACCCCAAAATTATTTATGAGCCACACCCACAGCTAA
CTAGAAATTTACCANAGATAGCACCTTCTGACCCAGTGCAGATTGGACGGTTAATTGGTAGGTTGGAGTTG
AGTCTCACGTTGAAACAGAATGAAGTGCTTCCTGGAGCTAAGTTGGATGTTGATGGACAGATAGACTCTAT
TCATCTACTCCTGTCACCAAGACAGGTGCACTTGCTTTTGGATATGTTGGCAGCTATTGCTGGACCANAAA
ATTCTANNNNAATAGGGTTAGCTAATAAAGATAGGAAAAATCNACCCATGCAGCAGGAAGACNAGTATCGA
ATTCAGA chromosomal mRNA ATG2B
SEQ ID NO: 319
TGTTCTTTGATATTAACTTGGATTCAGTTGAGCAGTCCTTAATATTTTGTATTAAACC
AAGTAACTTCAAATACAAGAAAATATT Unknown Function Chr7
SEQ ID NO: 320
TGGGTCGGGGTTTCGTACGTAGCAGAGCAGCTCCCTCGCTGCGATCTATTGAAAGTCA
GCCCTCGACACAAGGGTTTGT chromosomal 28S rRNA
SEQ ID NO: 321
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 322
CAGACCCCAGAAAAGGTGTTGGTTGATATAGACAGCAGGACGGTGGCCATGGAAGTCG
GAATCCGCTAAGGAGTGTGTAACAACTCACCTGCCGAATCAACTAGCCCTGAAAATGGATGGCGC
chromosomal 28S rRNA
SEQ ID NO: 323
TCACCACCACCACCATCATCACCACCACCACCNNTA
Unknown Origin
SEQ ID NO: 324
TCGGAAAAGTCATTTGATACTGTTAAACTAGAGTGTGGAAGAGGCTAT
Unknown Origin
SEQ ID NO: 325
GTTGGAACAATGTAGGTAAGGGAAGTCGGCAAGCCGGATCCGTAACTTCGGGATAAGG
ATTGGCTCTAAGGGC chromosomal 28S rRNA
SEQ ID NO: 326
GAAACAGATGATGAGAAGGACTCACTTAAGAAGCAGCTGAGAGAGA chromosomal
mRNA L0C100652789
SEQ ID NO: 327
TGTCCGTACAGAGCCGACGNCCCGGGCTTGATACTCCGACAGTGAGCCGTATCCAAGG
A bacterial origin

### Subject 1

SEQ ID NO: 328
GGAGCCTGAGAAACGGCTACCACATCCAAGGAAGGCAGCAGGCGCGCAAATTACCCAC
TCCCGACCCGGGGAGGTAGTGACGAAAAA chromosomal 18S rRNA
SEQ ID NO: 329
AGGGACGGCCGGGGGCATTCGTATTGCGCCGCTAGAGGTGAAATTCTTGGACCGGCGC
AAGACGGACCAGAGCGAAAGCATTTGCCAAGAATGTTTTCATTAATCAAGAACGAAAGTCGGAGGTTCGAA
GACGATCAGATACCGTCGTAGTTCCGACCATAAACGATGCCGACCGGCGATGCGGCGGCGTTATTCCCATG
ACCCGCCGGGCAGCTTCCGGGAAACCAAAGTCTTTGGGTTCCGGGGGGAGTATGGTTGCAAAGCTGAAACT
TAA chromosomal 18S rRNA
SEQ ID NO: 330
GCTTAACACAAAGCACCCAACTTACACTTAGGAGATTTCAACTTAACTTGACCGCTC
mitochondrial tRNA Valine
SEQ ID NO: 331
GCCATGGAAGTCGGAATCCGCTAAGGAGTGTGTAACAACTCACCTGCCGAATCAACTA
GCCCTGAAAATGGATGGCGC chromosomal 28S rRNA
SEQ ID NO: 332
GCTTAACACANNGCACCCAACTTACNCTTAGGAGATTTCANCTTANCTTGACCGCTC
mitochondrial tRNA Valine
SEQ ID NO: 333
TGCTTGGCTGAGGAGCCAATGGGGCGAAGCTACCATCTGTGGGATTATGACTGAACGC
CTCTAAGTCAGAATCCCGCCCAGG chromosomal 28S rRNA
SEQ ID NO: 334
AGGTCTCCAAGGTGAACAGCCTCTGGCATGTTGGAACAATGTAGGTAAGGGAAGTCGG
CAAGCCGGATCCGTAACTTCGGGATAAGGATTGGCTCTAAGGGC
chromosomal 28S rRNA
SEQ ID NO: 335
GCTTAACACAAAGCACCCAACTTACACTTAGGAGATTTCAACTTAACTTGACCGCTC
mitochondrial tRNA Valine
SEQ ID NO: 336
GCCCTATCAACTTTCGATGGTAGTCGCCGTGCCTACCATGGTGACCACGGGTGACGGG
GAATCAGGGTTCGATTCCGGAGAGGGAGCCTGAGAAACGGCTACCACATCCAAGGAAGGCAGCAGGCGCGC
AAATTACCCACTCCCGACCCGGGGAGGTAGTGACGAAAAATAAC chromosomal
18S rRNA
SEQ ID NO: 337
GGAGCCTGAGAAACGGCTACCACATCCAAGGAAGGCAGCAGGCGCGCAAATTACCCAC
TCCCGACCCGGGGAGGTAGTGACGAAAAATAAC chromosomal
18S rRNA
SEQ ID NO: 338
TGCTTGGCTGAGGAGCCAATGGGGCGAAGCTACCATCTGTGGGATTATGACTGAACGC
CTCTAAGTCAGAATCCCGCCCAGGCGGAACGATACGG chromosomal
28S rRNA
SEQ ID NO: 339
GGAGCCTGAGAAACGGCTACCACATCCAAGGAAGGCAGCAGGCGCGCAAATTACCCAC
TCCCGACCCGGGGAGGTAGTGACGAAAAATAACAATACAGGAC
chromosomal 18S rRNA
SEQ ID NO: 340
ACCGCGGTTCTATTTTGTTGGTTTTCGGAACTGAGGCCATGATTAAGAGGGACGGCCG
GGGGCATTCGTATTGCGCCGCTAGAGGTGGAATTCTTGGACCGGCGCAAGACGGACCAGAGCGAAAGCATT
TGCCAAGAATGTTTTCATTAATCAAGAACGAAAGTCGGAGGTTCGAAGACGATCAGATACCGTCGTAGTTC
CGACCATAAACGATGCCGACCGGCGATGCGGCGGCGTTATTCCCATGACCCGCCGGGCAGCTTCCGGGAAA
CCAAAGTCTTTGGGTTCCGGGGGGAGTATGGTTGCAAAGCTGAAACTTAAA
chromosomal 18S rRNA
SEQ ID NO: 341
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 342
AACACGGACCAAGGAGTCTAACACGTGCGCGAGTCGGGGGCTCGCACGAAAGCCGCCG
TGGCGCAATGAAGGTGAAGGCCGGCGCGCTCGCCGGCCGAGG
chromosomal 28S rRNA
SEQ ID NO: 343
GCTTAACACAAAGCACCCAACTTACACTTAGGAGATTTCAACTTAACTTGACCGCTC
mitochondrial tRNA Valine
SEQ ID NO: 344
TTGCACGGCGGAAAGCAATGCGACATTCTCACTTTGCGCTAATGCGCGTAGATCAACT
AATACCTGCTTACTTCTATCGATACTCTCTTTAGCATTAGTCTCGGGCAGCATAATATCAACATAGTCGAT
AACAACAAAGTCGAAGGTTATGCCAACAGCTTGGTACTTCTTTATAA
Unknown Origin
SEQ ID NO: 345
GGAGCCTGAGAAACGGCTACCACATCCAAGGAAGGCAGCAGGCGCGCAAATTACCCAC
TCCCGACCCGGGGAGGTAGTGACGAAAAATAACAATACAGGAC
chromosomal 18S rRNA
SEQ ID NO: 346
ATATAGACAGCAGGACGGTGGCCATGGAAGTCGGAATCCGCTAAGGAGTGTGTAACAA
CTCACCTGCCGAATCAACTAGCCCTGAAAATGGATGGCGC
chromosomal 28S rRNA
SEQ ID NO: 347
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 348
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTA
chromosomal 18S rRNA
SEQ ID NO: 349
AGGACCGCGGTTCTATTTTGTTGGTTTTCGGAACTGAGGCCATGATTAAGAGGGACGG
CCGGGGGCATTCGTATTGCGCCGCTAGAGGTGAAATTCTTGGACCGGCGCAAGACGGACCAGAGCGAAAGC
ATTTGCCAAGAATGTTTTCATTAATCAAGAACGAAAGTCGGAGGTTCGAAGACGATCAGATACCGTCGTAG
TTCCGACCATAAACGATGCCGACCGGCGATGCGGCGGCGTTGGAAAAAAAGGAGAAGA
chromosomal 18S rRNA
SEQ ID NO: 350
CAGGACGGTGGCCATGGAAGTCGGAATCCGCTAAGGAGTGTGTAACAACTCACCTGCC
GAATCAACTAGCCCTGAAAATGGATGGCGC
chromosomal 28S rRNA
SEQ ID NO: 351
AACACGGACCAAGGAGTCTAACACGTGCGCGAGTCGGGGGCTCGCACGAAAGCCGCCG
TGGCGCAATGAAGGTGAAGGCCGGCGC
chromosomal 28S rRNA
SEQ ID NO: 352
AAAGATGGTGAACTATGCCTGGGCAGGGCGAAGCCAGAGGAAACTCTGGTGGAGGTCC
GTAGCGGTCCTGACGTGCAAATCGGTCGTCCGACCTGGGTATAG
chromosomal 28S rRNA
SEQ ID NO: 353
TAGACAGCAGGACGGTGGCCATGGAAGTCGGAATCCGCTAAGGAGTGTGTAACAACTC
ACCTGCCGAATCAACTAGCCCTGAAAATGGATGGCGC
chromosomal 28S rRNA

### Subject 2

SEQ ID NO: 354
GGAATCCGCTAAGGAGTGTGTAACAACTCACCTGCCGAATCAACTAGCCCTGAAAATG
GATGGCGC chromosomal 28S rRNA
SEQ ID NO: 355
NNTCCAGTTTNNTNNCAACCANNCNGNGANGNNGNNGNNTNNNNNAAANNNNNTNCGA
GAGTATNNNNNTNNTNANCANNATGAAGANNNNCCGNNTGNNNCANCCNAACAANCCANCAATCACTNNGA
GAAACAAAAGNTTNNGNAACCCNGNCNTNNNNGAGAACNTNNTCAGTGGGATATNGGCATNGNCCANGCNG
TGAAAGCANNGAAAANGACTGGNGANGAAAGAATNGANCAGTATACTNTTATNNNCNNNATAAACANCNNG
AAGAGGNTTNNNNNCAAGCAAAAAAGANNNNNNCCTCCAAAACNNAANNTAAAAAAACCNGACGACCAAGA
TNTNTGCTGAATANTCAGCCAGANCAGACCANNNCNNGGGAGGTGGCNTCCTCACTNTCAAGTNNNGAAAT
TCGGAGACATANCCAGAGGCGGCACACAAGTNNGGANGAGGAAGAGCCNCCGCCTGTTAAAATANNCN
chromosomal mRNA WHSC1L1
SEQ ID NO: 356
TTACTTTGATAGAGTAAATAATAGGAGCTTAAACCCCCTTATTTCTA
mitochondrial tRNA Isoleucine
SEQ ID NO: 357
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 358
AATCCGCTAAGGAGTGTGTAACAACTCACCTGCCGAATCAACTAGCCCTGAAAATGGA
TGGCGC chromosomal 28S rRNA
SEQ ID NO: 359
ACTTGACCAACGGAACAAGTTACCCTAGGGATAACAGCGCAATCCTATTCTAGAGTCC
ATATCAACAATAGGGTTTACGACCTCGATGTTGGATCAGGACATCCCAATGGTGCAGC
mitochondrial 16S rRNA
SEQ ID NO: 360
GGAGCCTGAGAAACGGCTACCACATCCAAGGAAGGCAGCAGGCGCGCAAATTACCCAC
TCCCGACCCGGGGAGGTAGTGACGAAAAATAACAATAC
chromosomal 18S rRNA
SEQ ID NO: 361
NGNNNNTAACAAACCCACAGGTCCTAGNNNNNNNAACCTGNNTTAAAAATTTCGGTTG
GGGCGACCTCGGANCANAACCCANCCTCCNAGCANTACATGCTANNACTTCACCAGTCAAAGCGAACTACT
ATACTCAATTGATCCAATAACTTGACCAACGGANCNNGTTACCCTAGGGATAACAGCGCANTCCTATTCNN
NAGNCNNTATCAACAATAGGGTTTACNACCTCNATGTTGGATCAGGACNTCCCANTGGTGCANCCGCTATT
AAAGGTTCNNNNGNTCAACGATTAANGTCCTACGTGATCTGAGTTCANACCGGANTAATCCAGGTCGGTTT
CTATCTACTTCAAATTCCTCCCTGTACNAAAGGACAAGAGAAATAANGNCTACNTNNNNAAGCGCCTTCCC
CCNNANATGATATCATCTCNACTTANNATTATACCCNCACCCACCCNNNAACAGGGTTTGTTA
mitochondrial 16S rRNA
SEQ ID NO: 362
AAAAAATCCCAAACATATAACTGAACTCCTCACACCCAATTGGACCAATCTATCACCC
TATAGAAGAACTAATGTTAGTATAAGTAACATGAAAACATTCTCCTCCGCATAAGCCTGCGTCAGATTAAA
AC mitochondrial 16S rRNA
SEQ ID NO: 363
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 364
ATTACCACCATGCTCAGTAAGTCCATTTTTGCATGGAATATGGAGCCTTAAAACATGT
CATGAATTTGGAGTCCCTGGCACATAAATCTACCTTCAAATCAGAGGTCCTTAATGATGCCTAAACATACA
GTAAAATTAGAATCAGAAATACTTCTTTAAAAAATATTCAAAATGTGTTTGTTTCCCATGGGATTATTCTC
TATCCCACACGAATGTAAAAAAATCCACATTAATGATCCATTTAAGTATAGTTTTATTGGGTCCTTTTCTA
ATGATTAAAGGTTCTTTCTCAATTTCATTCCTCAGTCCTGCAAGTAAGGACTCATACTGAAGAGTACTGAA
ACAAGGACTTCTTGTCAGAAACAGCTTC chromosomal mRNA PIK3AP1
SEQ ID NO: 365
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 366
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 367
GCTTAACACAAAGCACCCAACTTACACTTAGGAGATTTCAACTTAACTTGACCGCTCT
GAC mitochondrial tRNA Valine
SEQ ID NO: 368
GGAATCCGCTAAGGAGTGTGTAACAACTCACCTGCCGAATCAACTAGCCCTGAAAATG
GATGGCGC chromosomal 28S rRNA
SEQ ID NO: 369
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACTATA
CTCAATTGATCCAATAACTTGACCAACGGAACAAGTTACCCTAGGGATAACAGCGCAATCCTATTCTAGAG
TCCATATCAACAATAGGGTTTACGACCTCGATGTTGGATCAGGACATCCCAA
mitochondrial 16S rRNA
SEQ ID NO: 370
TTGATATAGACAGCAGGACGGTGGCCATGGAAGTCGGAATCCGCTAAGGAGTGTGTAA
CAACTCACCTGCCGAATCAACTAGCCCTGAAAA
chromosomal 28S rRNA
SEQ ID NO: 371
GCTTAACACAAAGCACCCAACTTACACTTAGGAGATTTCAACTTAACTTGACCGCTC
mitochondrial tRNA Valine
SEQ ID NO: 372
GGGGGCATTCGTATTGCGCCGCTAGAGGTGAAATTCTTGGACCGGCGCAAGACGGACC
AGAGCGAAAGCATTTGCCAAGAATGTTTTCATTAATCAAGAACGAAAGTCGGAGGTTNGAAGACGATCAGA
TACCGTCGTAGTTCCGACCATAAACGATGCCGACCGGCGATGCGGCGGCGTTATTCCCATGACCCGCCGGG
CAGCTTCCGGGAAACCAAAGTCTTTGGGTTCCGGGGGGAGTATGGTTGCAAAGCTGAAACTTAA
chromosomal 28S rRNA
SEQ ID NO: 373
GGAATCCGCTAAGGAGTGTGTAACAACTCACCTGCCGAATCAACTAGCCCTGAAAATG
GATGGCGC chromosomal 28S rRNA
SEQ ID NO: 374
CCCAAACATATAACTGAACTCCTCACACCCAATTGGACCAATCTATCACCCTATAGAA
GAACTAATGTTAGTATAAGTAACATGAAAACATTCTCCTCCGCATAAGCCTGCGTCAGATTAAAA
mitochondrial 16S rRNA
SEQ ID NO: 375
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 376
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCA mitochondrial 16S rRNA
SEQ ID NO: 377
GTTGATATAGACAGCAGGACGGTGGCCATGGAAGTCGGAATCCGCTAAGGAGTGTGTA
ACAACTCACCTGCCGAATCAACTAGCCCTGAAAATGGATGGCGC
chromosomal 28S rRNA
SEQ ID NO: 378
TGTGACGAAAAATAACAATACAGGACTCTTTCGAGGCCCTGTAATNGGAATGAGTCCA
CTTTAAATCCTTTAACGAGGATCCAT
chromosomal 18S rRNA
SEQ ID NO: 379
GGATCCGTAACTTCGGGATAAGGATTGGCTCTAAGGGC
chromosomal 28S rRNA

### Subject 4

SEQ ID NO: 380
AATTCCAAAAGAATNCATCACACGNNTNGTNTNNNACCNGAAACACAAAACCCTTGCT
TTAATTAAAGATGGCCGTGTTATNGGTGGTATCTGTTTCNNNNNNTTCCCATNTCANGGATTCACAGAGAT
TGTCTTNTGTGCTGTAACCTCAAATGAGCAAGTCAAGGGTTANGGANCACACNTGANGAATCATTTGAAAG
AATATCACATAANNCNNGACATCNTGAACTTCCTCNCATATGCAGANGAATANGCAATTGGATACTTTAAG
AAACAGGGTTTCTCCAAAGAAATTAAAATACCTAAAACCAAATATGTTGGNTATATCAAGGATTATGAAGG
AGCCACTTTAATGGGATGTGAGCTAAATCCACGGATCCCGTACACNNAATTTTCTGTCATCATTAAAAAGC
AGAAGGAGATAATTAAAAAACTGATTGAAAGAAAACAGGCACAAATTCGAAAAGTTTACCCTGGACTNTCA
TGTTTNAAAGAN chromosomal mRNA KAT2B
SEQ ID NO: 381
TAACTGAACTCCTCACACCCAATTGGACCAATCTATCACCCTATAGAAGAACTAATGT
TAGTATAAGTAACATGAAAACATTCTCCTCCGCATAAGCCTGCGTCAGATTA
mitochondrial 16S rRNA
SEQ ID NO: 382
ACTCAATTGATCCAATAACTTGACCAACGGAACAAGTTACCCTAGGGATAACAGCGCA
ATCCTATTCTAGAGTCCATATCAACAATAGGGTTTACGACCTCGATGTTGGATCAGGACATCCCGA
mitochondrial 16S rRNA
SEQ ID NO: 383
CAGACCCCAGAAAAGGTGTTGGTTGATATAGACAGCAGGACGGTGGCCATGGAAGTCG
GAATCCGCTAAGGAGTGTGTAACAACTCACCTGCCGAATCAACTAGCCCTGAAAATGGATGGCGC
chromosomal 28S rRNA
SEQ ID NO: 384
GGANCAATCTATCACCCTATAGAAGAACTAATGTTAGTATAAGTAACATGAAAA
mitochondrial 16S rRNA
SEQ ID NO: 385
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 386
GCTTAACACAAAGCACCCAACTTACACTTAGGAGATTTCAACTTAACTTGACCGCTCT
GA mitochondrial tRNA Valine
SEQ ID NO: 387
CCCATAGTAGGCCTAAAAGCAGCCACCAATTAAGAAAGCGTTCAAGCTCAACACCCAC
TACCTAAAAAATCCCATCA mitochondrial 16S rRNA
SEQ ID NO: 388
GCCATGGAAGTCGGAATCCGCTAAGGAGTGTGTAACAACTCACCTGCCGAATCAACTA
GCCC chromosomal 28S rRNA
SEQ ID NO: 389
AAAGAATACATCACACGGNTNGTNTNTGACCCGAAACACAAAACCCTTGCTTTAATTA
AAGATGGCCGTGTTATTGGTGGTATCTGTTTCCGTATGTTCCCATNTCAAGGATTCACAGAGATTGTNTTN
TGTGCTGTAACCTCAAATGAGCAAGTCAAGGGNTATGGAACACACNTGATGAATCATTTGAAAGAATATCA
CATAAAGCATGACATCCTGAACTTCCTCACATATGCAGATGAATATGCAATTGGATACTTTAAGAAACAGG
GTTTCTCCAAAGAAATTAAAATACCTAAAACCAAATATGTTGGCTATATCAAGGATTATGAAGGAGCCACT
TTAATGGGATGTGAGCTAAATCCACGGATCCCGTACACAGAATTTTCTGTCATCATTAAAAAGCAGAAGGA
GATAATTAAAAAACTGATTGAAAGAAAACAGGCACAAATTCGAAAAGTTTACCCTGGACTTTCATGTTTTA
AAGA chromosomal mRNA KAT2B
SEQ ID NO: 390
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 391
GGGCGTAAAGGATGCGTAGGCTGGAAATCAAGTCGAAAGTGAAATCCAACGGCTCAAC
CGTTGAACTGCTTTCGAAACTGGTTACCTAGAATATGGGAGAGGTAGA
bacterial origin
SEQ ID NO: 392
GAGAAAGCTCACAAGAACTGCTAACTCATGCCCCCATGTCTAACAACATGGCTTTCTC
A mitochondrial tRNA Serine 2
SEQ ID NO: 393
ATCCCAAACATATAACTGAACTCCTCACACCCAATTGGACCAATCTATCACCCTATAG
AAGAACTAATGTTAGTATAAGTAACATGAAAACATTCTCCTCCGCATAAGCCTGCGTCAGATTAAAACACT
GAACTGACAATTAACAGCCCAATATCTACAATC mitochondrial 16S rRNA
SEQ ID NO: 394
TGAAAAACCATTTCATAACTTTGTCAAAGTTAAATTATAGGCTAAATCCTATATA
mitochondrial tRNA Aspartate
SEQ ID NO: 395
TATNCTTGCTGTTGAGTCTCCNNACCCTGANGCTANGANATNACTANCANGGNTCNNN
GNACANATAAAAACTTCNNATTCATGGNCCCGCAACACAACAGCNTNNNNANGAGGGATTTCNACATCCCC
ATCCACTTCCNTCNTATCNGTATGATTATTTGCTATANNATGTGCTCCATTCNCCTCCCCNTTTGCTGTGT
TTTCNCCNTTTTTTGCAGATNCTTGTTGGNTGGCTGCANCTGCGGNNGNTGCANCANCTGCTGCCTGTTGC
TGNGCAANNNNANCTCTATANNNNTGTTGNCTTGNNNGNACTACTTCNNGNNNNNCNNNNTCNATCNNGNA
CNNANACTCTATTGNTCNACCNNCANNNNNNGNACCATNNNNNNNANTACTANACTTCTGCTTCNACNNNC
TGNANACCTTTCTGGATGATANAAANCAATGCTGCGGA
chromosomal mRNA TBL1XR1
SEQ ID NO: 396
TAACTGAACTCCTCACACCCAATTGGACCAATCTATCACCCTATAGAAGAACTAATGT
TAGTATAAGTAACA mitochondrial 16S rRNA
SEQ ID NO: 397
GGAGCCTGAGAAACGGCTACCACATCCAAGGAAGGCAGCAGGCGCGCAAATTACCCAC
TCCCGACCCGGGGAGGTAGTGACGAAAAATAACAATACAGGA
chromosomal 18S rRNA
SEQ ID NO: 398
AATCCGCTAAGGAGTGTGTAACAACTCACCTGCCGAATCAACTAGCCCTGAAAATGGA
TGGCGC chromosomal 28S rRNA
SEQ ID NO: 399
GCTGCGGGATGAACCGAACGCCGGGTTAAGGCGCCCGATGCCGACGCTCATCAGACCC
chromosomal 28S rRNA
SEQ ID NO: 400
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 401
CCTTGGTGCCCGAGTGCCTTGGTGCCCGAGTGTAGAATCTTAGTTCAACTTTAAATTT
GCCCACAGAACCCTCTAAATCCCCTTGTAAATTTAACTGTTAGTCCAAAGAGGAACAGCTCTTTGGACACT
AGGAAAAAACCTTGTAGAGAGAGTAAAAAATTTAACACCCATAGTAGGCCTAAAAGCAGCCACCAATTAAG
AAAGCGTTCAAGCTCAACACCCACTACCAAAAAACAAAAAA
mitochondrial 16S rRNA
SEQ ID NO: 402
ANCACGGNCCAAGGNGTCTAACACGTGCGCGAGTCGGGGGCTCGCACGAAAGCCGCCG
TGGCGCAATGAAGGTGAAGGCCGGCGCGCTCGCCGGCCGAGG
chromosomal 28S rRNA

### Subject 5

SEQ ID NO: 403
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 404
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 405
GGAGCCTGAGAAACGGCTACCACATCCAAGGAAGGCAGCAGGCGCGCAAATTACCCAC
TCCCGACCCGGGGAGGTAGTGACGAAAAATAACAATAC
chromosomal 18S rRNA
SEQ ID NO: 406
GTCTACGGCCATACCACCCTGAACGCGCCCGATCTCGTCTGATCTCGGAAGCTAAGCA
GGGTCGGGCCTGGTTAGTACTTGGATGGGAGACCGCCTGGGAATACCGGGTGCTGTAGGCTA
chromosomal 5S rRNA
SEQ ID NO: 407
TGCTGGTTGGTCTGGTGATGAATGTTCACGGTGCAGGGGGCAGCCTTGAGCAGGTCGG
TAAAATTATGCTGC bacterial origin
SEQ ID NO: 408
ATATAGACAGCAGGACGGTGGCCATGGAAGTCGGAATCCGCTAAGGAGTGTGTAACAA
CTCACCTGCCGAATCAACTAGCCCTGAAAA chromosomal
28S rRNA
SEQ ID NO: 409
TGCTTGGCTGAGGAGCCAATGGGGCGAAGCTACCATCTGTGGGATTATGACTGAACGC
CTCTAAGTCAGAATCCCGCCCAGG chromosomal 28S rRNA
SEQ ID NO: 410
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 411
AATCCGCTAAGGAGTGTGTAACAACTCACCTGCCGAATCAACTAGCCCTGAAAATGGA
TGGCGC chromosomal 28S rRNA
SEQ ID NO: 412
TGATATAGACAGCAGGACGGTGGCCATGGAAGTCGGAATCCGCTAAGGAGTGTGTAAC
AACTCACCTGCCGAATCAACTAGCCCTGAAAATGGATGGCGC
chromosomal 28S rRNA
SEQ ID NO: 413
GCCATGGAAGTCGGAATCCGCTAAGGAGTGTGTAACAACTCNCNNNNNGAATCAACTA
GCCCTGAAAATGGATGGCGC chromosomal 28S rRNA
SEQ ID NO: 414
GCCCTATCAACTTTCGATGGTAGTCGCCGTGCCTACCATGGTGACCACGGGTGACGGG
GAATCAGGGTTCGATTCCGGAGAGGGAGCCTGAGAAACGGCTACCACATCCAAGGAAGGCAGCAGGCGCGC
AAATTACCCACTCCCGACCCGGGGAGGTAGTGACGAAAAATAAC
chromosomal 18S rRNA
SEQ ID NO: 415
GAAAAATAACAATNCAGGACTNNTTNGAGNCCCTGTAANNNGAATGAGTNCACTTTAA
NTCNNTTAACGAGGATNCATTGGAGGGCAANTCTGGTNCCAGCAGCCGCGGTAATTCCAGCTCCAATAGCG
TATATTAAAGNNGNNNCAGTTAAAAAGCTNGTAGTTGGATCTTGGGNNNTGGCGGGCGGTCCGCCGCGAGG
CGAGCCACCGCCCGTCCCCNGCCCCTTGCCTCTCGGCGCCCCCTCGATGCTCTTAGCTGAGTGTCCCGCGG
GGCCCGAAGCGTTTACTTTGAAAAAATTAGAGTGTTCAAAGCAGGCCCGAGCCGCCTGGATACCGCAGCTA
GGAATAATGGAATAGGACCGCGGTTCTATTTTGTTGGTTTTCGGAACTGAGGCCATGATTAAGAGGGACGG
CCGGGGGCATTCGTATTGCGCCGCTAGAGGTGAAATTCTTGGACCGGCGCAAGACGGACCAGAGCGAAAGC
ATTTGCCAAGAATGTTTTCATTAATCAAGAACGAAAGTCGGAGGTTCGAAGACGATCAGATACCGTCGTAG
TTCCGACCATAAACGATGCCGACCGGCGATGCGGCGGCGTTATTCCCATGACCCGCCGGGCAGCTTCCGGG
AAACCAAAGTCTTTGGGTTCCGGGGGGAGTATGGTTGCAAAGCTGAAACTTAA
chromosomal 18S rRNA
SEQ ID NO: 416
AGAAAAGGTGTTGGTTGATATAGACAGCAGGACGGTGGCCATGGAAGTCGGAATCCGC
TAAGGAGTGTGTAACAACTCACCTGCCGAATCAACTAGCCCTGAAAATGGATGGCGC
chromosomal 28S rRNA
SEQ ID NO: 417
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 418
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAA
chromosomal 18S rRNA
SEQ ID NO: 419
TTAGTGACGCGCATGAATGGATTAACGAGATTCCCACTGTCCCTATCTACTATCTAGC
GAAACCACAGCGAAGGGAACGGGCTTCGCAAAATCAGCGGGGAAAGAAGACCCTGTTGAGCTTGACTCTAG
TTTGACATTGTGAAAAGACATAGGGGGTGTAGAATAGG
chromosomal 28S rRNA
SEQ ID NO: 420
TTAGACCGTCGTGAGACAGGTTAGTTTTACCCTACTGATGATGTGTTGTTGCCATGGT
AATCCTGCTCAGTACGAGAGGAACCGCAGGTTCAGACATT
chromosomal 28S rRNA
SEQ ID NO: 421
TAGGGACCTGTATGAATGGCTCCACGAGGGTTCAGCTGTCTCTTACTTTTAACCAGTG
AAATTGACCTGCCCGTGAAGAGGCGGGCATAACACAGCAAGACGAGAAGACCCTATGGAGCTTTAATTTAT
TAATGCAAACAGTACCTAACAAACCCACAGGTCCTAAACTACCAAACCTGCATTAAAAATTTCGGTTGGGG
CGACCTCGGAGCAGAACCCAACCTCCGAGCAGTACATGCTAAGACTTCACCAGTCAAAGCGAACTACTATA
CTCAATTGATCCAATAACTTGACCAACGGAACAAGTTACCCTAGGGATAACAGCGCAATCCTATTCTAGAG
TCCATATCAACAATAGGGTTTACGACCTCGATGTTGGATCAGGACATCCCAATGGTGCAGCCGCTATTA
mitochondrial 16S rRNA
SEQ ID NO: 422
GTACGTAGCAGAGCAGCTCCCTCGCTGCGATCTATTGAAAGTCAGCCCTCNNNNNAAG
GNNNNGAATTNTCGGNCACCAANC
chromosomal 28S rRNA
SEQ ID NO: 423
AGACCCCAGAAAAGGTGTTGGTTGATATAGACAGCAGGACGGTGGCCATGGAAGTCGG
AATCCGCTAAGGAGTGTGTAACAACTCACCTGCCGAATCAACTAGCCCTGAAAATGGATGGCGC
chromosomal 28S rRNA
SEQ ID NO: 424
GGTGCATGGCCGTTCTTAGTTGGTGGAGCGATTTGTCTGGTTAATTCCGATAACGAAC
GAGACTCTGGCATGCTAACTAGTTACGCGAC
chromosomal 18S rRNA
SEQ ID NO: 425
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 426
TAGTCCGGATTGGAGTCTGCAACTCGACTCCATGAAGTCGGAATCGCTAGTAATCG
bacterial origin
SEQ ID NO: 427
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGG chromosomal 18S rRNA
SEQ ID NO: 428
GCCATGGAAGTCGGAATCCGCTAAGGAGTGTGTAACAACTCACCTGCCGAATCAACTA
GCCCTGAAAATGGATGGCGC chromosomal 28S rRNA
SEQ ID NO: 429
TCGTTGGCGGGTTATAAATGTCTCCTTCTCCCTGTGATTTGTTTAATCCGTGGAAATG
GTGCTGGTCCTATGTAAACAAGCCAAGTGCGGAATGAAGGCAGTCACCCATGCGTGGCCAGCCTGCCTATT
TGTCAGAAAACCTTCATAAATACTGAGCTGGGGCTGGGCAAGG Unknown
Function Chr5
SEQ ID NO: 430
GCTAAACCTAGCCCCAAACCCACTCCACCTTACTACCAGACAACCTTAGCCAAACCAT
TTACCCAAATAAAGTATAGGCGATAGAAATTGAAACCTGGCGCAATAGATATAGTACCGCAAGGGAAAGA
mitochondrial 16S rRNA

### Subject 6

SEQ ID NO: 431
GTTGAATGAAAATCGCAGTCAGTGTGGCTTTGGTAGTCTAACAGTCAATCAGAATCTT
AACCTTACAGCAATGAATCATGCCAACTATATGGCATCGGTAACTGAAACAAATAAACAGCCATTTGCAAG
TCACGAAGAGCAAGCTGAAACGGGTTTGTTAGATACAGGAATTACCAACCCCTATTATTCAGGTATTGATT
TAACCACTAGACTAAACCCCTTTA Unknown Origin
SEQ ID NO: 432
TTGATATAGACAGCAGGACGGTGGCCATGGAAGTCGGAATCCGCTAAGGAGTGTGTAA
CAACTCACCTGCCGAATCAACTAGCCCTGAAAA
chromosomal 28S rRNA
SEQ ID NO: 433
TCGGAATCCGCTAAGGAGTGTGTAACAACTCACCTGCCGAATCAACTAGCCCTGAAAA
chromosomal 28S rRNA
SEQ ID NO: 434
TCGGAATCCGCTAAGGAGTGTGTAACAACTCACCTGCCGAATCAACTAGCCCTGAAAA
chromosomal 28S rRNA
SEQ ID NO: 435
GGACCCCCCCCAACACAAAGCCCCTGTCCCGACCCCCAACTCTGAN
chromosomal mRNA NPR1
SEQ ID NO: 436
TTCCGCGGTGCCGTGGCGCAGCGCGCGCAGGTTGCGGCCGATGGTCGCCTCCTCGTCG
TGCGCCGGGATCACCACGCTCGCTACGGAC Unknown Origin
SEQ ID NO: 437
CTTAACACAAAGCACCCAACTTACACTTAGGAGATTTCAACTTAACTTGACCGCTC
mitochondrial tRNA Valine
SEQ ID NO: 438
TTACTTTGATAGAGTAAATAATAGGAGCTTAAACCCCCTTATTTCTA
mitochondrial tRNA Isoleucine
SEQ ID NO: 439
CCACTACCACCACCACCACCACCACTACTACCACCACCACCACCACCACTACCACCAC
CACCACCACTACCACCACCACCACTAC Unknown Origin
SEQ ID NO: 440
ATCTGTACCATTCCACTCCATTCCATTTCATTCCATTCCACTCCACTCCACTCCATTC
CATTGCGTTCCATTCCACTCCACTACACTCCATTCCTTTCCTTTCCTTTCCATTCCACTCCATTCCATTCC
ATTA Unknown Origin
SEQ ID NO: 441
TGTTCCATTCCATTCCATTCCATTCCATTCCATTNNNTNNCATTCCACTCCATTCCAC
TCCATTCCATTCCACTCCATTACATTCCATTCCATTCCACTCCATTCCATTCCACTCCATTCCATTCCATT
CCATTCCACTCCATTCCACTCCACTCCA Unknown Origin
SEQ ID NO: 442
TTGGTGGTAGTAGCAAATATTCAAACGAGAACTTTGAAGGCCGAAG
chromosomal 28S rRNA
SEQ ID NO: 443
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 444
GCCCAGAGACCAGACCCCCCCAAAGGACCAGACCCCCCCACAGGGACCCAGAGACCAG
CCACCCCCACACAGGG Unknown Function Chr10
(spliced)
SEQ ID NO: 445
CCACCACCACCATCACCATCACCACCACCACCACCNCCNNCATCACCACCACCACCAT
CACCACC Unknown Origin
SEQ ID NO: 446
GGTTTGAGCCTCAGATTCGTAGAATAGTCGAACAAGATACTATGCCTCCAAAGGGTGT
CCGCCACACTATGATGTTTAGTGCTACTTTTCCTAAGGAAATACAGATGCTGGCTCGTGATTTCTTAGATG
AATATATCTTCTTGGCTGTAGGAAGAGTTGGCTCTACCTCTGAAAACATCACACAGAAAGTAGTTTGGGTG
GAAGAATCAGACAAACGGTCATTTCTGCTTGACCTCCTAAATGCAACAGGCAAGGATTCACTGACCTTAGT
GTTTGTGGAGACCAAAAAGGGTGCAGATTCTCTGGAGGATTTCTTATACCATGAAGGATACGCATGTACCA
GCATCCATGGAGACCGTTCTCAGAGGGATAGAGAAGAGGCCCTTCACCAGTTCCGCTCAGGAAAAAGCCCA
ATTTTAGTGGCTACAGCAGTAGCAGCAAGAGGACTGGACATTTCAAATGTGAAACATGTTATCAATTTTGA
CTTGCCAAGTGATATTGAAGAAT chromosomal mRNA DDX3X
SEQ ID NO: 447
TTTCCGCCAGACGTGGGCCAAAATCCGGGAACTGTTTAGGATTAGCTTCCAGGCT
bacterial origin
SEQ ID NO: 448
TCACCATCACCACCACCACCACCATCACCACCACCATCACTACCACCACCACCACCAT
CA Unknown Function Chr1
SEQ ID NO: 449
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 450
ATCTGTACCATTCCACTCCATTCCATTTCATTCCATTCCACTCCACTCCACTCCATTC
CATTGCGTTCCATTCCACTCCACTACACTCCATTCCTTTCCTTTCCTTTCCATTCCACTCCATTCCATTCC
ATTA Unknown Origin
SEQ ID NO: 451
GNATAAGGACCGCGGTTCTATTTTGTTGGTTTTCGGAACTGAGGCCATGATTAAGAGG
GACGGCCGGGGGCATTCGTATTGCGCCGCTAGAGGTGAAATTCTTGGACCGGCGCAAGACGGACCAGAGCG
AAAGCATTTGCCAAGAATGTTTTCATTAATCAAGAACGAAAGTCGGAGGTTCGAAGACGATCAGATACCGT
CGTAGTTCCGACCATAAACGATGCCGACCGGCGATGCGGCGGCGTTATTCCCATGACCCGCCGGGCAGCTT
CCGGGAAACCAAAGTCTTTGGGTTCCGGGGGGAGTATGGTTGCAAAGCTGAAACTTAAA
chromosomal 18S rRNA
SEQ ID NO: 452
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 453
ACTACCACCACCACCACCACCACCACCAACCATCACCATC
Unknown Function Chr7
SEQ ID NO: 454
TTCCATTCCATTCCNTTCCATTCCATTCCATTCCATTCCATTCCACTCCATTCCACTC
CATTCCATTCCACTCCATTACATTCCATTCCATTCCACTCCATTCCATTCCACTCCATTCCATTCCATTCC
ATTCCACTCCATTCCACTCCACTCCA Unknown Origin
SEQ ID NO: 455
CTACCACCACCACCACCACCATCACTACCACCACCACCACCACCATCACTACCACCAC
CACCACCACCACCACCATCACTACCACCACCACCACCACCATCACTACCACC
Unknown Function Chr22
SEQ ID NO: 456
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 457
AATTCCNTGNGTTGGGGGCCTGGGCTCANGACNGANGGGGCN
Unknown Origin
SEQ ID NO: 458
GAATAGGACCGCGGTTCTATTTTGTTGGTTTTCGGAACTGAGGCCATGATTAAGAGGG
ACGGCCGGGGGCATTCGTATTGCGCCGCTAGAGGTGAAATTCTTGGACCGGCGCAAGACGGACCAGAGCGA
AAGCATTTGCCAAGAATGTTTTCATTAATCAAGAACGAAAGTCGGAGGTTCGAAGACGATCAGATACCGTC
GTAGTTCCGACCATAAACGATGCCGACCGGCGATGCGGCGGCGTTATTCCCATGACCCGCCGGGCAGCTTC
CGGGAAACCAAAGTCTTTGGGTTCCGGGGGGAGTATGGTTGCAAAGCTGAAACTTAAA
chromosomal 18S rRNA
SEQ ID NO: 459
ATCTGTACCATTCCACTCCATTCCATTTCATTCCATTCCACTCCACTCCACTCCATTC
CATTGCGTTCCATTCCACTCCACTACACTCCATTCCTTTCCTTTCCTTTCCATTCCACTCCATTCCATTCC
ATTA Unknown Origin
SEQ ID NO: 460
TCGGAATCCGCTAAGGAGTGTGTAACAACTCNNCNGNNNNNTCAACTAGCCCTGAAAA
chromosomal 28S rRNA

1. A method for diagnosing a disease or other medical condition in a subject comprising:
a. isolating a microvesicle fraction from a biological sample from a subject;
b. measuring the levels of one or more tRNAs; and
c. correlating the levels of said tRNAs to the presence or absence of the disease or other medical condition.

2. A method for prognosing a disease or other medical condition in a subject comprising:
a. isolating a microvesicle fraction from a biological sample from a subject;
b. measuring the levels of one or more tRNAs; and
c. correlating the levels of said tRNAs to a higher susceptibility to or predisposition to develop the disease or other medical condition.

3. A method for monitoring a disease or other medical condition in a subject comprising:
a. isolating a microvesicle fraction from a biological sample from a subject;
b. measuring the levels of one or more tRNAs; and
c. correlating the levels of said tRNAs to a higher susceptibility or predisposition to develop the disease or other medical condition.

4. The method of claim 1, wherein said tRNA is a chromosomal tRNA, a mitochondrial tRNA, or fragments or combinations thereof.

5. The method of claim 1, further comprising measuring the levels of one or more HERV elements.

6. The method claim 1, wherein the disease or medical condition is associated with increased or decreased levels of one or more tRNAs, HERV sequences, or fragments or combinations thereof, when compared to a control sample.

7. The method of claim 6, wherein the control sample is a biological sample from a subject without the disease or medical condition.

8. The method of claim 1, wherein the biological sample is chosen from a tissue sample or a bodily fluid sample.

9. The method of claim 8, wherein the bodily fluid sample is plasma or serum.
